(19) United States
(12) Patent Application Publication
(10) Pub. No.: US 2011/0268822 A1
(43) Pub. Date: Nov. 3, 2011

(54) METHODS FOR SYNTHESIZING KOTALANOL AND STEREOISOMERS AND ANALOGUES THEREOF, AND NOVEL COMPOUNDS PRODUCED THEREBY

(75) Inventors: Brian Mario Pinto, Coquitlam (CA); Jayakanthan Kumarasamy, Burnaby (CA); Ravindranath Nasi, Ottawa (CA); Sankar Mohan, Burnaby (CA)

(73) Assignee: SIMON FRASER UNIVERSITY, Burnaby, BC (CA)

(21) Appl. No.: 12/934,898

(22) PCT Filed: Mar. 25, 2009
(86) PCT No.: PCT/CA09/00397

§ 371 Date: Apr. 20, 2011

Related U.S. Application Data
(60) Provisional application No. 61/039,192, filed on Mar. 25, 2008.
(60) Provisional application No. 61/146,531, filed on Jan. 22, 2009.
(60) Provisional application No. 61/150,672, filed on Feb. 6, 2009.

(51) Int. Cl. A61K 36/00; A61K 31/40; A61K 31/381; G01N 27/447; C07D 207/00; C07D 333/46; A61P 1/00; A61P 3/10; A61K 31/33; C07D 345/00

(52) U.S. Cl. 424/725; 514/183; 514/425; 514/445; 540/1; 548/556; 549/66; 204/451

(57) ABSTRACT

Compounds having the general formula (I): wherein X is S, Se or NH, and stereoisomers thereof, and de-O-sulfonated analogues of all of the foregoing, but excluding naturally occurring kotalanol and de-O-sulfonated kotalanol, and methods for synthesizing same. The compounds are useful as glycosidase inhibitors, and may be used in the treatment of diabetes. The synthetic compounds may also be used as standards in the calibration or grading of natural or herbal remedies produced from natural sources of glycosidase inhibitors such as kotalanol.

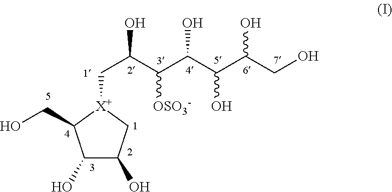

### REFERENCE TO RELATED APPLICATIONS

This application claims the benefit of U.S. provisional patent application No. 61/039,192 filed 25 Mar. 2008, No. 61/146,531 filed 22 Jan. 2009, and No. 61/150,672 filed 6 Feb. 2009, each of which is incorporated by reference herein.

### TECHNICAL FIELD

This application relates to methods for synthesizing kotalanol and de-O-sulfonated kotalanol, as well as stereoisomers and analogues thereof potentially useful as glycosidase inhibitors.

### BACKGROUND

Glycosidases are responsible for the processing of complex carbohydrates which are essential in numerous biological recognition processes.¹ Inhibition of these glycosidases can have profound effects on quality control, maturation, transport, and secretion of glycoproteins, and can alter cell-cell or cell-virus recognition processes.

This principle is the basis for the potential use of glycosidase inhibitors for the treatment of various disorders and diseases such as diabetes, cancer, and other viral diseases;^{2,3} for example, acarbose, a pseudotetrasaccharide, and voglibose, an aminocyclitol, are inhibitors of α-glucosidases and have been approved for the clinical treatment of diabetes.^{4,5} Glycosidase inhibitors have also proved useful in the investigation of disorders such as Gaucher's disease.⁶ An attractive approach to potent glucosidase inhibitors is to create compounds that mimic the oxacarbenium ion-like transition state of the enzyme-catalyzed reaction.^{7,8}

Many of the natural and synthetic azasugars are believed to mimic the transition state in either charge or shape, thus making them good glycosidase inhibitors.⁹ They are presumed to be partially protonated in the active site at physiological pH, thus providing the stabilizing electrostatic interactions between the inhibitor and the carboxylate residues in the enzyme active site. An alternative approach to carbohydrate mimics is to replace the ring oxygen atom of carbohydrates with other heteroatoms such as sulfur and selenium. Indeed, sulfonium salts are known to be quite stable, and have been proposed as mimics of the oxacarbenium ion-like transition state.¹⁰

Some sulfonium ions with glucosidase inhibitory properties occur naturally. For example, Yoshikawa et al. discovered a new class of glycosidase inhibitors, namely salaprinol 1,¹¹ salacinol 2,¹² ponkoranol 3,¹¹ and kotalanol 4¹³ from the plant Salacia reticulata, all of which possess a common sulfonium ion stabilized with an internal sulfate counterion and differing only in the number of carbons in the polyhydroxylated side chain (see Chart 1 below). Recently, Ozaki et al.¹⁴ isolated another α-glucosidase inhibitor from the same plant and assigned its structure to a 13-membered cyclic sulfoxide; this structure has been reassigned by Yoshikawa et al.¹⁵ to be the de-O-sulfonated kotalanol 5. The latter compound was shown by Ozaki et al.¹⁴ to be the most active compound against rat intestinal glucosidase in this series of compounds (compare the Kᵢ values for salacinol (0.97, 0.20, and 1.1 μM), kotalanol (0.54, 0.42, and 4.2 μM), and de-O-sulfonated kotalanol (0.11, 0.05, and 0.42 μM) using maltose, sucrose, and isomaltose as substrates, respectively).

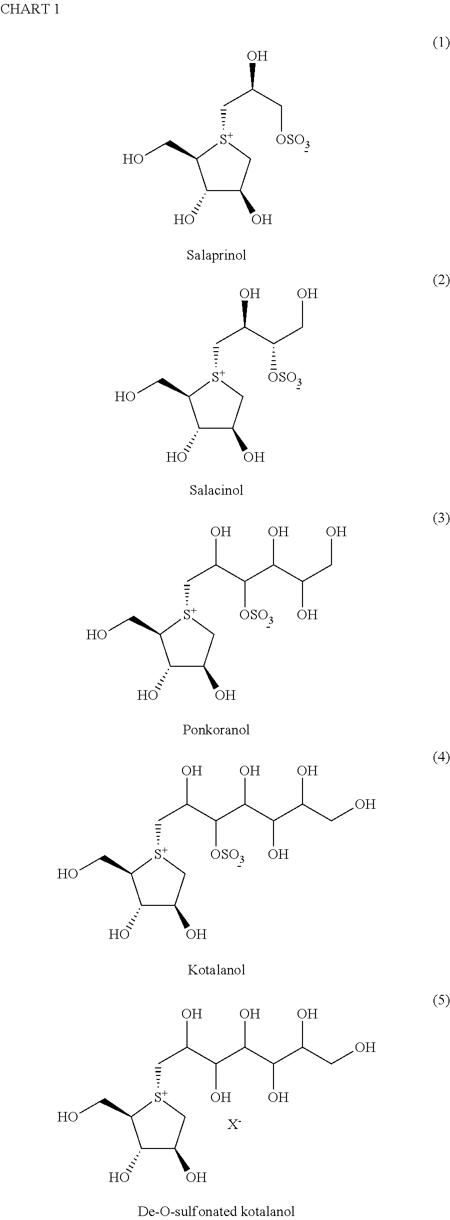

The aqueous extracts of the roots and stems of the plant Salacia reticulata have been traditionally used in the Ayurvedic system of Indian medicine for the treatment of Type-2 diabetes. Recent clinical trials on human patients with Type-2 diabetes mellitus using the aqueous extract of the same plant have indicated good glycemic control and side effects comparable to the placebo control group.¹⁶ The Salacia reticulata plant is, however, in relatively small supply and is not readily available outside of Sri Lanka and India. Accordingly, it would be desirable if kotalanol 4 and its analogues could be produced synthetically in good yield.

The inventors and others have carried out extensive research on the synthesis of salacinol 2 and higher homologues, differing in stereochemistry at the stereogenic centers, and congeners in which the sulfur heteroatom has been substituted by the cognate atoms nitrogen and selenium.¹⁷However, prior to the present work, the precise stereochemical structure of kotalanol 4 had not yet been determined, nor had a convenient method of its synthesis.

There accordingly remains a need for a convenient synthesis of the naturally-occurring compound kotalanol 4 in reasonable yield. There further remains a need for novel analogues of kotalanol which may be more effective or selective inhibitors of glycosidases, and convenient methods for synthesizing these compounds.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In accordance with the invention, compounds having the general structures I and III are provided, such compounds excluding naturally occurring kotalanol and de-O-sulfonated kotalanol:

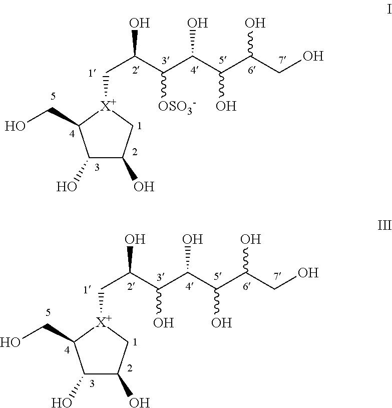

wherein X is selected from the group consisting of S, Se and NH.

Methods for synthesizing kotalanol and de-O-sulfonated kotalanol, as well as stereoisomers and analogues thereof are also provided. In one embodiment, a method for direct synthesis of kotalanol is provided using a cyclic sulfate derived from D-perseitol.

The invention also encompasses use of the compounds of the invention for inhibition of glycosidases, such as intestinal glycosidases. In one particular embodiment, the invention relates to a method of treating diabetes by administering to an affected patient a therapeutically effective amount of a compound of the invention.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a representation of the molecular structure of compound 23 as determined by single-crystal X-ray structure analysis.

FIG. 2 is a comparison of the ¹H NMR spectra of compounds 17 and 18; (A) compound 17 in D₂O; (B) compound 17 in pyridine-d₅; (C) compound 18 in D₂O; (D) compound 18 in pyridine-dₛ.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

### 1.0 INTRODUCTION

Kotalanol 4 is a naturally occurring compound which may be extracted from the roots and stems of Salacia reticulata, a plant native to Sri Lanka and India. This application relates to synthetic routes for preparing kotalanol 4 and its nitrogen and selenium analogues and stereoisomers thereof having the general structure I shown below, wherein X may be S, Se, or NH. This application also relates to the preparation of de-O-sulfonated kotalanol, its nitrogen and selenium analogues, and stereoisomers thereof.

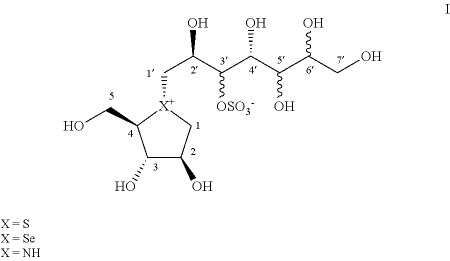

Chemical degradation studies have indicated that the 1-deoxy-4-thiopentofuranosyl portion of kotalanol 4 is identical to that in salacinol 2.¹³ However, the absolute configuration of the stereogenic centers in the heptitol side chain and at the sulfur center had not previously been determined. The inventors' previous synthetic work has yielded several 5-carbon- and 6-carbon-chain analogues as well as selenium congeners 6-15 (Chart 2) which have been screened for inhibitory activity against recombinant human maltase glucoamylase (MGA), a critical intestinal glucosidase involved in the breakdown of glucose oligomers into glucose. Several of the compounds showed inhibitory activity in the low micromolar range (Table 1). The stereochemistry at the different stereogenic centers on the side chain appears to play a significant role in biological activity. It appears that the compounds containing the S-configuration at C-2′, the R-configuration at C-4′, and the S-configuration at C-5′ are the most active in the sulfur series of compounds. The inventors note, however, that in the selenium series the activities of the selenium analogues, 11 and 15 (0.10 and 0.14), suggest that the stereochemistry at C-5′ could be R. The stereochemistry at C-3′-appears to be unimportant, although the stereochemistry may be inferred to be S, to reflect a presumed common biosynthetic pathway as salacinol.

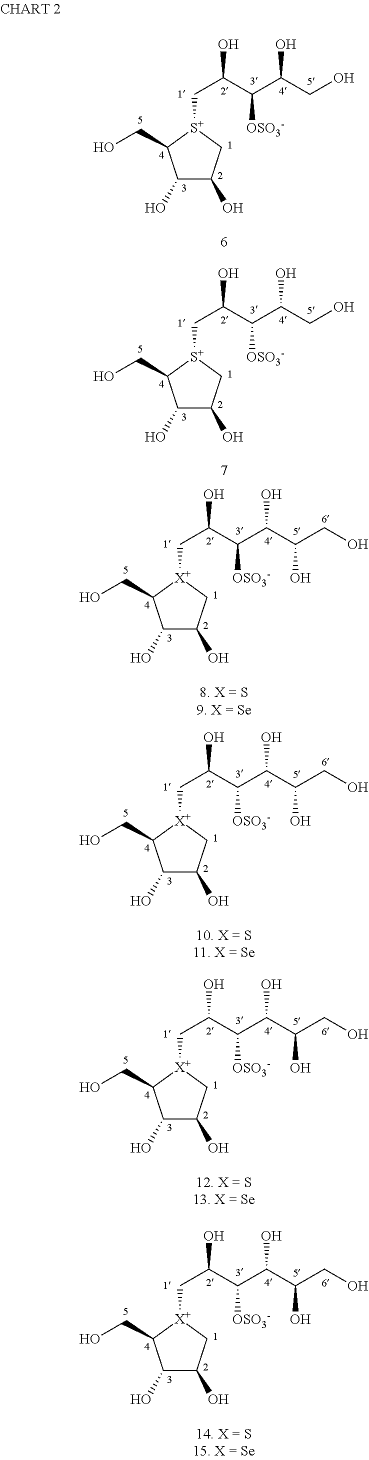

TABLE 1
Experimentally determined Kᵢ values.^{a}

| | Stereochemistry of the acyclic side-chain | | | | |
|---|---|---|---|---|---|
| Inhibitor | C-2′ | C-3′ | C-4′ | C-5′ | Kᵢ (μM) |
| 6 | S | R | S | — | NA^{b,18} |
| 7 | S | S | R | — | 0.26 ± 0.02¹⁸ |
| 8 | S | R | R | S | 0.25 ± 0.02¹⁸ |
| 9 | S | R | R | S | 0.10 ± 0.02¹⁹ |
| 10 | S | S | R | S | 0.17 ± 0.03¹⁸ |
| 11 | S | S | R | S | 0.10 ± 0.02¹⁹ |
| 12 | R | S | R | R | NA^{b,20} |
| 13 | R | S | R | R | 41.0 ± 7.0²⁰ |
| 14 | S | S | R | R | 0.65 ± 0.10²¹ |
| 15 | S | S | R | R | 0.14 ± 0.03²¹ |
| Salacinol | S | S | — | — | 0.19 ± 0.02²² |
| Blintol | S | S | — | — | 0.49 ± 0.05²² |

^{a}Analysis of MGA inhibition was performed using maltose as the substrate, and measuring the release of glucose. Absorbance measurements were averaged to give a final result;
^{b}NA: not active.

It is noteworthy that each of the seven carbon analogues (Chart 3, 16) recently synthesized by Muraoka et al.²³ with the S-configuration at C-4′ showed less inhibitory activity than natural kotalanol 4, also suggesting that the necessary stereochemistry at C-4′ is R.

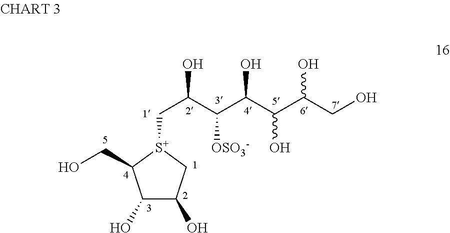

A recent report from Yoshikawa et al. describes the isolation from Salacia prinoides of a six-carbon chain analogue of salacinol, ponkoranol, that shows IC₅₀ values against maltase, sucrase, and isomaltase in the low micromolar range.²³ Comparison of physical data to those of the inventors' previous synthetic derivatives^{18,20,21} confirms that ponkoranol is indeed compound 10 (Chart 2). A U.S. patent application also describes a six-carbon chain analogue isolated from Salacia reticulata named reticulanol.²⁴ Comparison of the physical data indicate once again that this compound is also compound 10 above.

### 2.0 SYNTHESIS OF STEREOISOMERS AND ANALOGUES OF KOTALANOL

As described below, the inventors have elucidated the precise stereochemistry of kotalanol 4 and have developed stereoisomers and analogues potentially useful as glycosidase inhibitors. Based on the finding of the precise stereochemistry, the inventors have also developed a convenient synthesis for naturally occurring kotalanol 4.

The general synthetic scheme for synthesizing analogues and stereoisomers of kotalanol is set forth in Scheme I. Analogues and stereoisomers of kotalanol may be de-O-sulfonated as set forth in Scheme II.

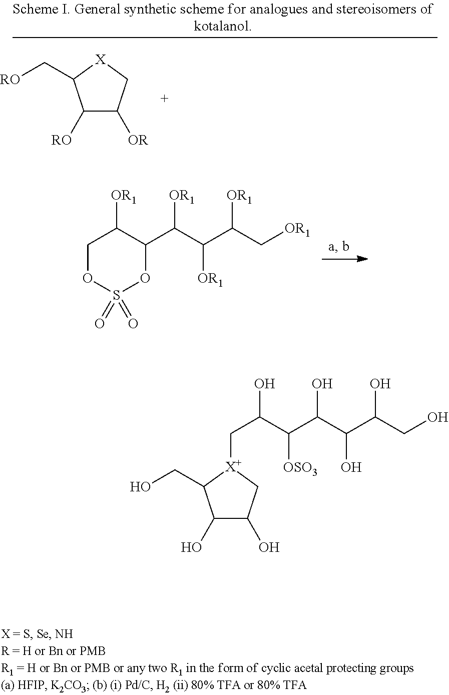

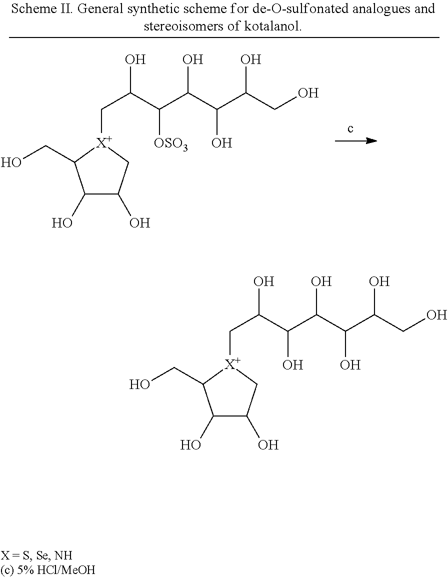

Several structures were synthesized to determine the stereochemical structure of kotalanol 4. These include compounds 17 and 18, which have the S-configuration at C-2′ and C-3′, the R-configuration at C-4′, the S-configuration at C-5′, and either the S- or R-configuration at C-6′ (Chart 4); and compounds 19 and 20, with the S-configuration at C-2′ and C-3′, the R-configuration at C-4′ and C-5′, and either the S- or R-configuration at C-6′ (Chart 4).

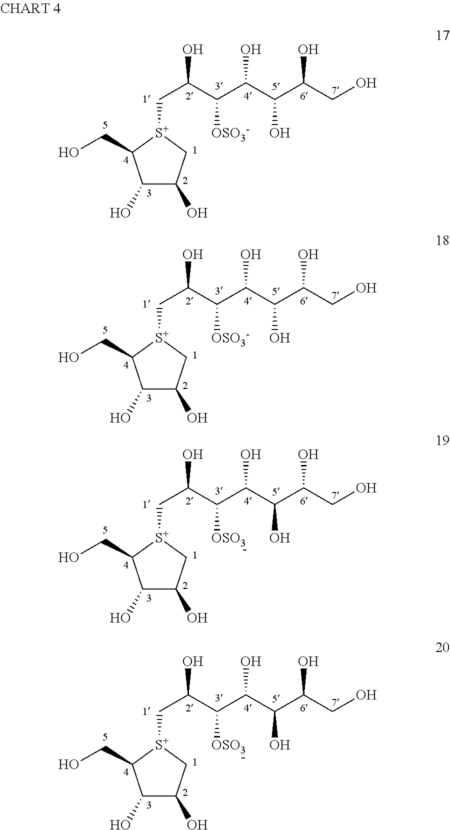

The strategy developed to synthesize compounds 17 and 18 involves alkylation of the anhydrothioalditol 21¹⁰ at the heteroatom by a cyclic sulfate derivative, specifically, the tri-O-benzyl-butane-2,3-diacetal-heptyl-1,3-cyclic sulfates 22 and 23 (see retrosynthetic analysis in Scheme 1, below). The inventors' previous experience suggests that selective attack of the heteroatom at the least hindered primary center will occur. The butane-2,3-diacetal (BDA) unit as a protecting group has been used extensively in the total synthesis of natural products,²⁵ and the inventors have used it in the synthesis of lower homologues.²⁶ Relatively strong acidic conditions are required for its removal, thus permitting the selective removal of the benzylidene group in B prior to installation of the 1,3-cyclic sulfate in A. Intermediate B could be obtained from C via asymmetric dihydroxylation, which could, in turn, be obtained from the D-glucose derivative D by a Wittig reaction (Scheme 1).

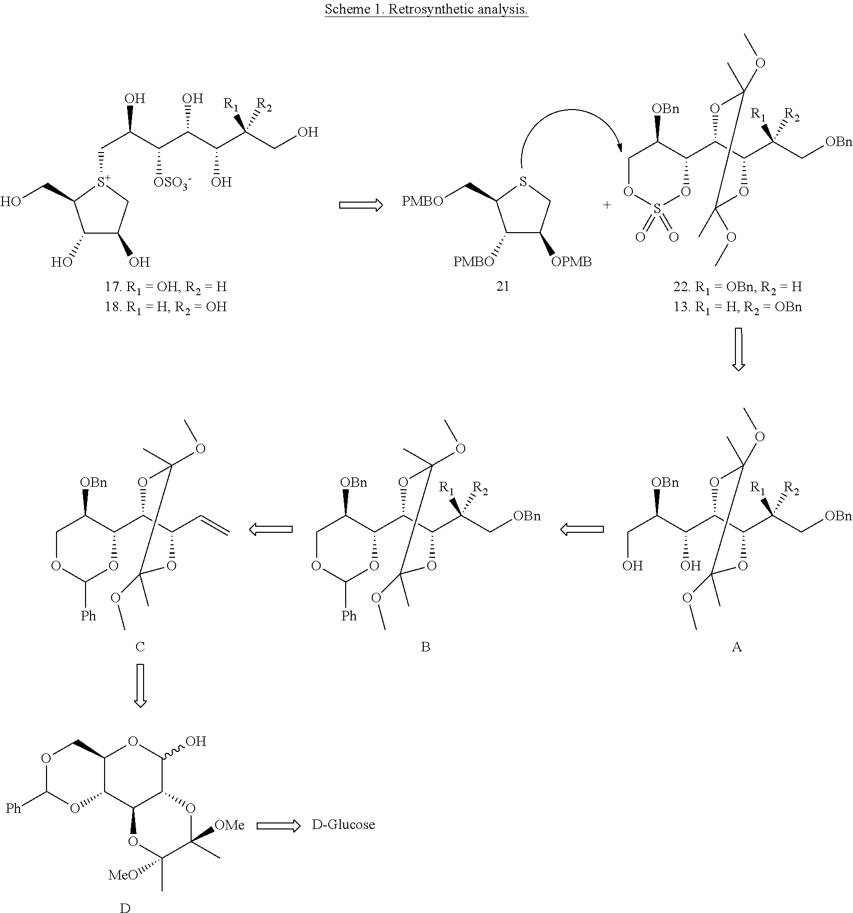

The preparation of 24 is unprecedented in the literature and was successfully synthesized from D-glucose via a three-step sequence (Scheme 2). Thus, allyl D-glucopyranoside was treated with 2,3-butanedione and trimethylorthoformate in the presence of camphorsulfonic acid (CSA) in boiling methanol to give an inseparable mixture of 2, 3- and 3, 4-BDA-protected intermediates. This mixture was reacted directly with benzaldehyde dimethylacetal in presence of a catalytic amount of PTSA to yield the fully protected, and separable derivative 24 in 31% overall yield. Isomerization of the allyl glucoside 24 was effected with t-BuOK in DMF, and subsequent cleavage of the resulting enol ether using I₂ in THF:H₂O gave the 2,3-BDA-4,6-O-benzylidene-D-glucopyranose 25. Treatment of this hemiacetal with methyltriphenylphosphonium bromide provided the olefinic product 26 (83%), which was benzylated to afford compound 27.

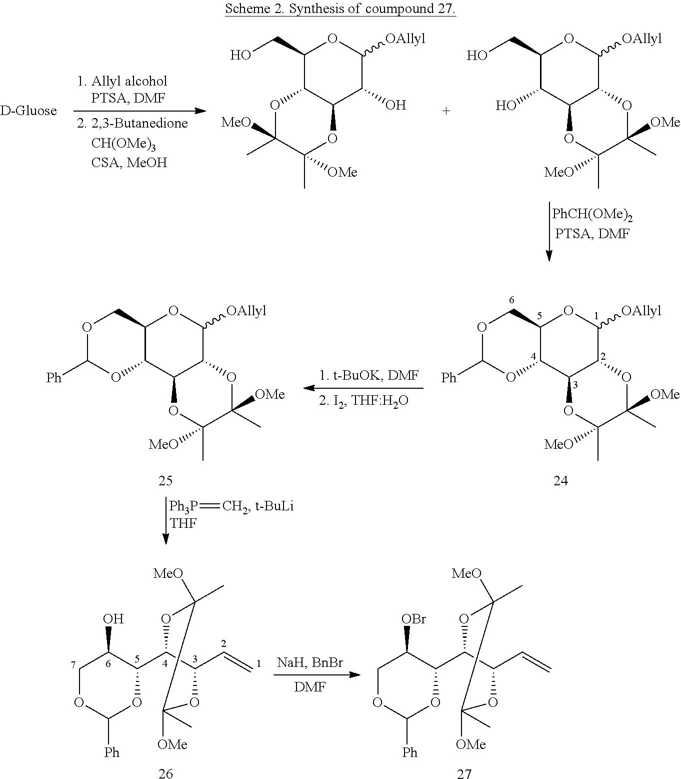

With compound 27 in hand, the inventors next sought to introduce the two hydroxyl groups. The OsO₄-catalyzed dihydroxylation of 27 proceeded smoothly in an acetone-water mixture with N-methylmorpholine-N-oxide (NMO) as reoxidant. One diastereoisomer 28 was obtained exclusively under these reaction conditions (Scheme 3, Table 2). The stereochemical outcome of this dihydroxylation follows Kishi's empirical rule, which predicts that in the syn-hydroxylation of acyclic allylic alcohols the relative stereochemistry between the preexisting hydroxyl group and the adjacent newly introduced hydroxyl group in the major product is erythro.²⁷ The syn-hydroxylation from the same side of the allylalkoxy group, which is sterically more compressed, affords the minor product. Kishi's rule has previously been shown to apply in the dihydroxylation of a variety of carbohydrate allylic systems.²⁸

Compound 28 was benzylated under standard conditions to give 30, which was then subjected to mild methanolysis using catalytic PTSA in methanol to effect selective removal of the benzylidene group (Scheme 4) and give the corresponding diol 31 in 73% yield. The cyclic sulfate 22 was then obtained by treatment of 31 with thionyl chloride and triethylamine followed by oxidation with sodium periodate and ruthenium (III) chloride as a catalyst (Scheme 4).

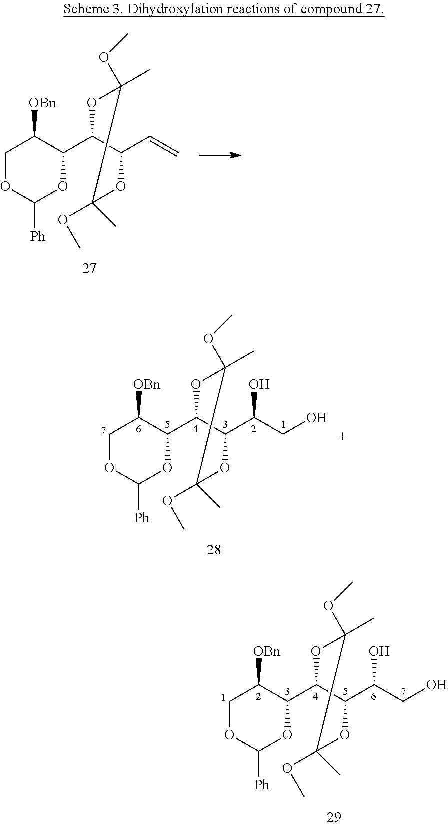

TABLE 2

| Entry | Compound | Conditions | Product | Yield (%) | dr^{a} |
|---|---|---|---|---|---|
| 1 | 27 | OsO₄, NMO | 28 | 93 | 20:1 |
| 2 | 27 | AD-mix β | 28:29 | 90 | 7:3 |
| 3 | 27 | AD-mix α | 28 | 91 | 20:1 |

^{a}Determined by 500 MHz ¹H NMR.

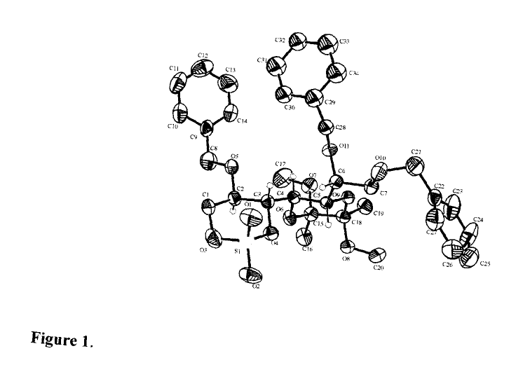

The inventors next examined the asymmetric dihydroxylation reaction using commercially available AD-mix β under the reported standard conditions (AD-mix (3 in a 1:1 mixture of tert-BuOH—H₂O). However, a separable 7:3 diastereomeric mixture (28 and 29) was obtained in which compound 28 was still the predominant isomer (Table 2). The corresponding asymmetric dihydroxylation of 27 using AD-mix-α, with the intention of obtaining the distereoisomer of compound 28, was examined next. The AD-mix-α afforded compound 28 exclusively (Scheme 3). The unsatisfactory selectivity in the dihydroxylation reaction can probably be attributed to unfavorable steric interactions between the bulky dihydroxylating reagent and the BDA protecting group, situated next to the olefinic reactive site. The stereochemistry at the C-6 position in compound 28 was therefore inverted by the Mitsunobu protocol to obtain the desired diol 29. Accordingly, selective protection of the primary hydroxyl group using tert-butyldimethylsilylchloride gave 32 in 91% yield, which when treated under standard Mitsunobu conditions afforded the ester 33 (Scheme 4). Removal of the p-nitrobenzoyl and tert-butyldimethylsilyl groups using sodium methoxide and tetrabutylammonium fluoride, respectively, gave the diol 29. Compound 29 was obtained as a colorless crystalline solid, suitable for single-crystal X-ray analysis, that established conclusively the absolute configurations at the newly generated stereogenic center. With the diol in hand, the cyclic sulfate 23 was synthesized following the same reaction sequence as discussed above for the synthesis of 22. The structure of the cyclic sulfate 23 was also confirmed by single crystal X-ray analysis (FIG. 1). The cyclic sulfates 22, 23 were thus assigned the structures: 1, 2,6-tri-O-benzyl-3,4-O-(2′,3′-dimethoxybutane-2,3′-diyl)-D-glycero-D-gulitol-5,7-cyclic sulfate and 2,6,7-tri-O-benzyl-4,5-O-(2′,3′-dimethoxybutane-2′,3′-diyl)-D-glycero-L-gulitol-1,3-cyclic sulfate, respectively.29

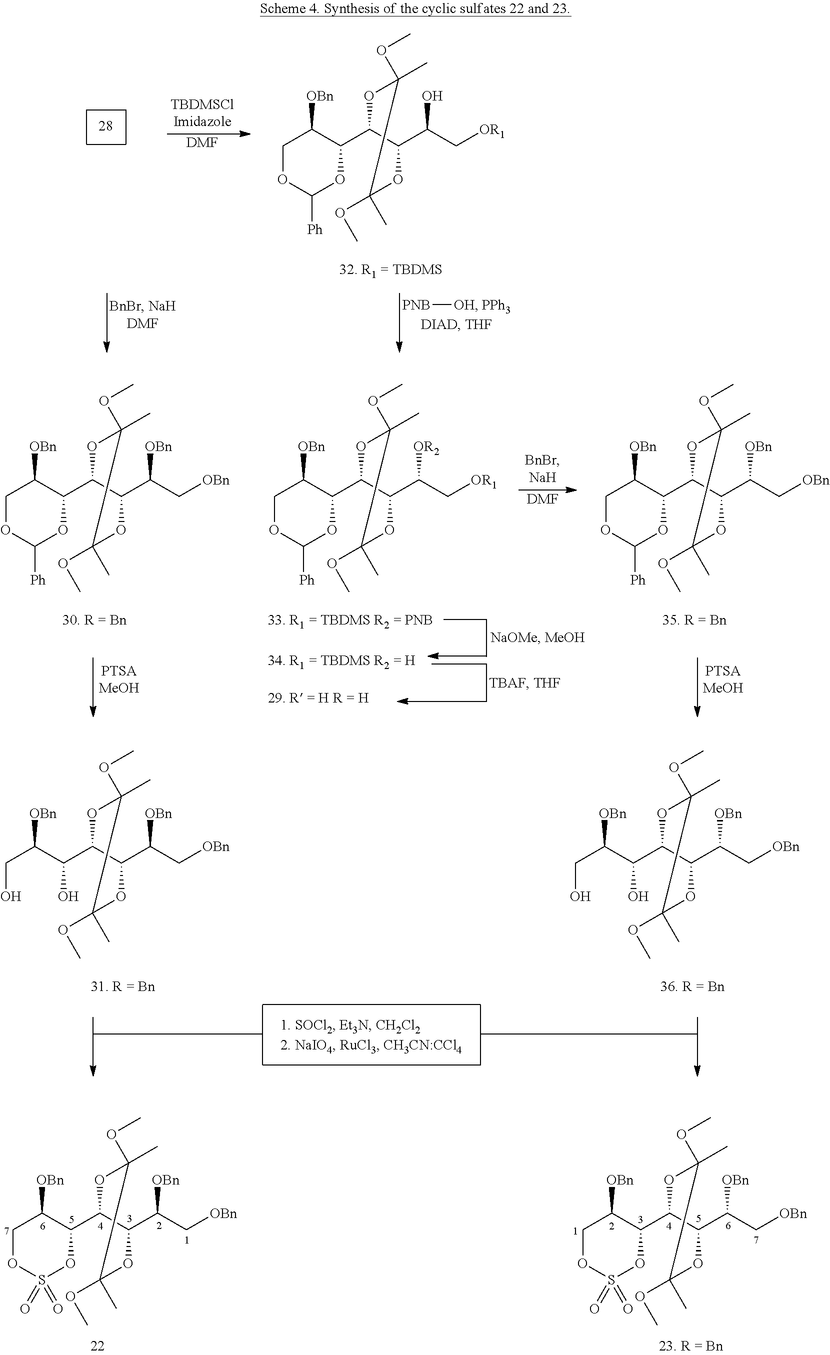

The coupling reactions of the cyclic sulfate 22 with the protected thioarabinitol were investigated next. 2,3,5-Tri-O-p-methoxybenzyl-1,4-anhydro-4-thio-D-arabinitol 21³⁰ was prepared by a method analogous to that developed for the synthesis of the corresponding selenium derivative.³¹ The reaction of the thioarabinitol 21 with the cyclic sulfate 22 was found to proceed very slowly at 72° C. The inventors also observed that longer reaction time led to decomposition of the coupling product. The coupling reaction was therefore terminated before complete consumption of the starting materials. The protected sulfonium sulfate 37 was obtained as the sole product in 55% yield using 1,1,1,3,3,3-hexafluoro-2-propanol (HFIP) as solvent (Scheme 5). The lack of ring strain accounts partially for the observed slow reactivity of the cyclic sulfate; in contrast, the inventors' earlier studies with cyclic sulfates in which the torsional strain was released by opening of the cyclic sulfate led to favorable alkylation reactions.¹⁰ Deprotection of the coupled product 37 was performed with two successive reactions. The sulfonium salt 37 was first treated with Pd/C/H₂ in aqueous acetic acid to effect hydrogenolytic cleavage, followed by treatment with trifluoroacetic acid to yield the desired zwitterionic compound 17 (Scheme 5).

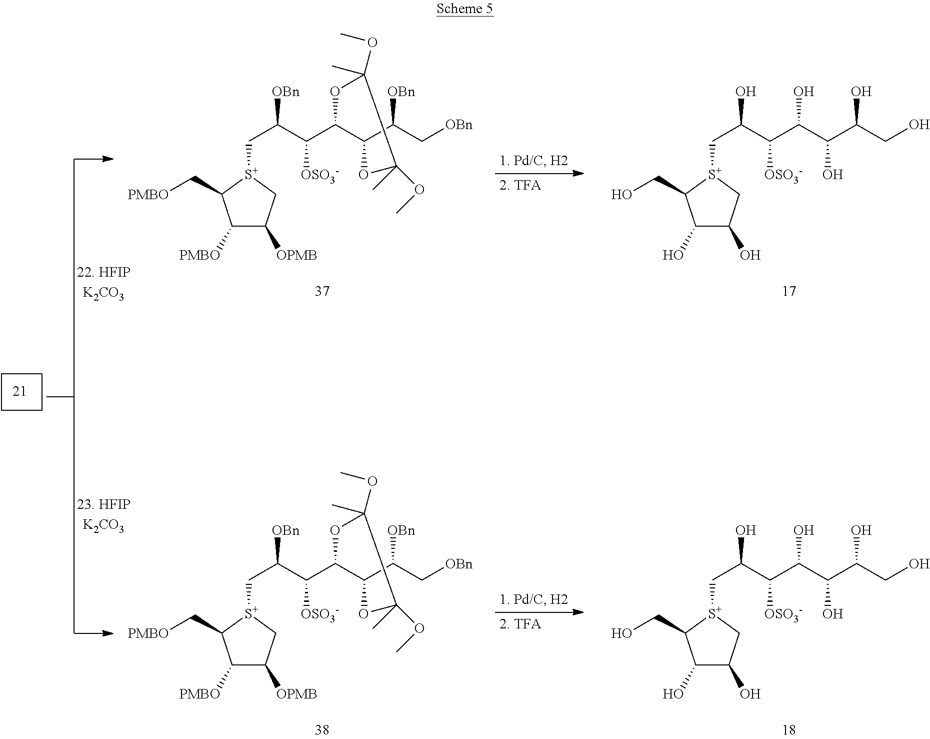

Analogously, the cyclic sulfate 23 was reacted with the thioether 21 at 72° C. for 48 h in HFIP to give 38 in 61% yield. Compound 38 was then deprotected, as above, to afford the desired zwitterionic compound 18 (Scheme 5).

Retrosynthetic analysis indicated that the two analogues 19 and 20 (Chart 4) could be obtained by alkylation of a thioether with terminal 1,3-cyclic sulfates (Scheme 6). The heptitol-derived cyclic sulfates could be synthesized, in turn, from a hexose by successive Wittig and asymmetric dihydroxylation reactions. The desired stereochemistry at C-2′-C-5′ could be readily introduced from D-mannitol.

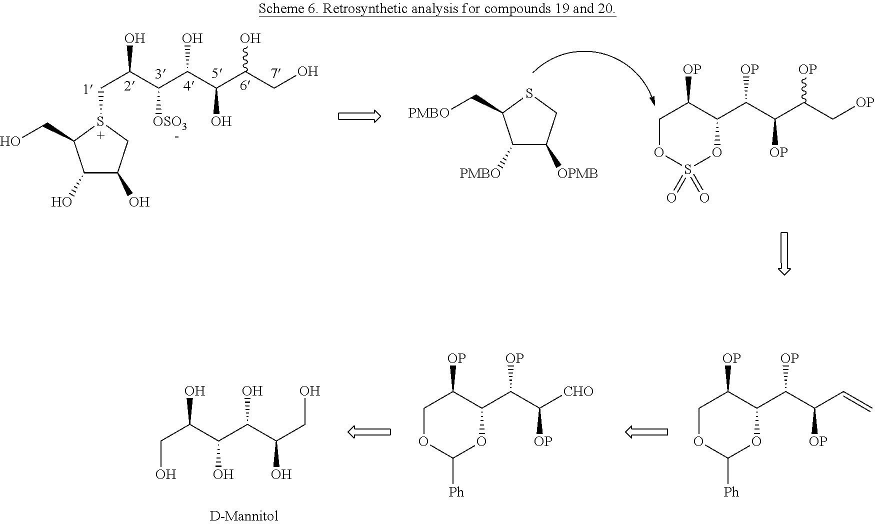

The inventors' initial attempts employed the cyclic sulfates 39 and 40 (Chart 5), but intramolecular ring opening of the cyclic sulfate moiety by one of the benzyl ethers caused decomposition. Therefore, some rigidity was introduced to the cyclic sulfate through protecting groups in order to avoid the intramolecular ring opening reaction. The inventors' previous work also suggested that release of torsional strain in the cyclic sulfate led to increased reactivity.²⁰ Accordingly, the inventors chose the methylene acetal (see 41) as a protecting group which could survive the acidic conditions required for removal of the benzylidene acetal prior to installation of the cyclic sulfate; the methylene acetal can be introduced under strongly basic conditions.

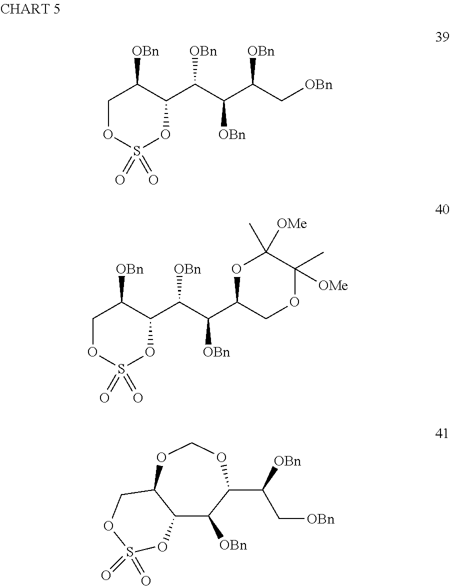

Thus, di-O-benzylidene-D-mannitol 42,³² was treated with dibromomethane in the presence of aqueous sodium hydroxide and tetra-n-butylammonium bromide as catalyst;³³ removal of one of the benzylidene groups using catalytic p-toluenesulfonic acid (PTSA) in methanol then gave the diol 43³⁴ in 65% yield over two steps (Scheme 7). In the deprotection reaction, owing to the C-2 symmetric nature of compound 42, removal of either benzylidene group led to the same diol, 43. The primary hydroxyl group was selectively protected with tert-butyldimethylsilyl chloride (TBDMS) followed by protection of the secondary hydroxyl group as its benzyl ether. Finally, the silyl protecting group was removed using tetra-n-butylammonium fluoride to yield 44 in 62% yield over three steps. Oxidation of the alcohol 44 using Dess-Martin periodinane gave the aldehyde which was treated with methyltriphenylphosphonium bromide to yield the olefin 45 in 56% yield over two steps.

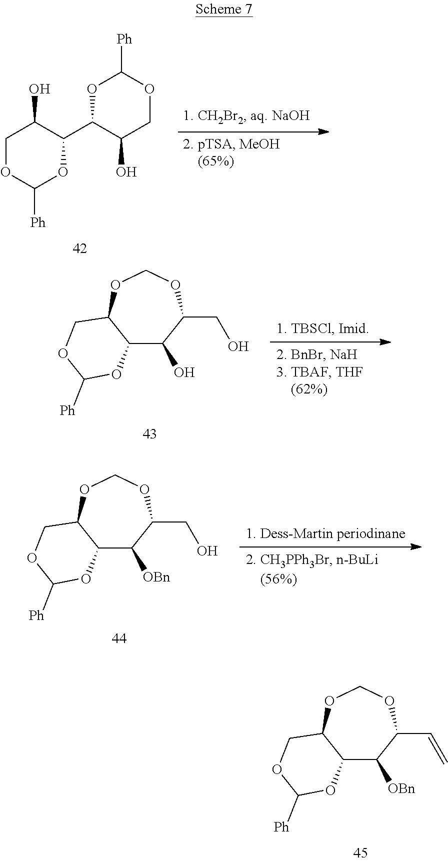

Kishi's empirical rule for dihydroxylation of acyclic allylic alcohols³⁵ suggests that, treatment of the olefin 45 with OsO₄ should yield the syn-dihydroxylated product, with the erythro configuration between the pre-existing hydroxyl group and the newly generated hydroxyl group. Sharpless asymmetric dihydroxylation³⁶ using AD-mix-β should offer the other diastereomer. Thus, treatment of the olefin 45 under OsO₄-catalyzed dihydroxylation conditions gave a diastereomeric ratio of 7:1, with the major isomer 46 in 84% yield (Scheme 8). The major isomer was separated by column chromatography and then the hydroxyl groups were protected as benzyl ethers. To introduce the cyclic sulfate moiety, the benzylidene group was first removed using catalytic PTSA in methanol, and the resulting diol 47 was then treated with thionyl chloride and triethylamine, followed by oxidation of the corresponding cyclic sulfite with sodium periodate and ruthenium (III) chloride as a catalyst to give the cyclic sulfate 48 in 61% yield.

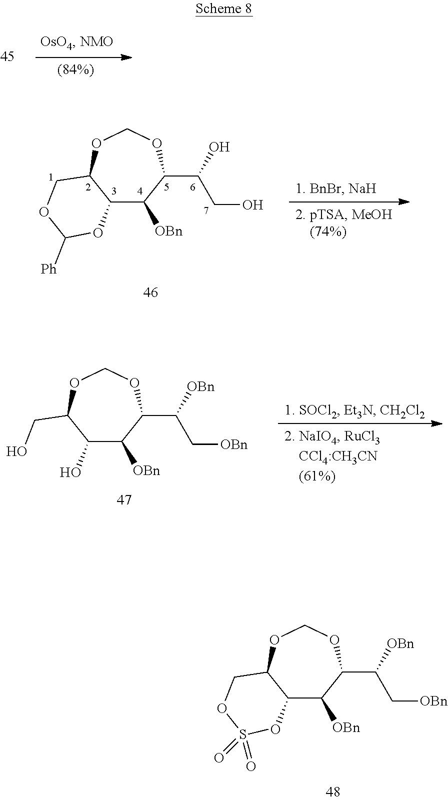

In order to prove the stereochemistry at the newly formed stereogenic center (C-6) of compound 46, it was converted into the tri-cyclic derivative 49 as shown in Scheme 9. The observed coupling constant (J_{4.6}=10.2 Hz) between protons H-5 and H-6 confirms their di-axial relationship and thus proves the configuration at C-6 as being R. The assignment was corroborated by the observed NOE contact between H-4 and H-6 (Scheme 9).

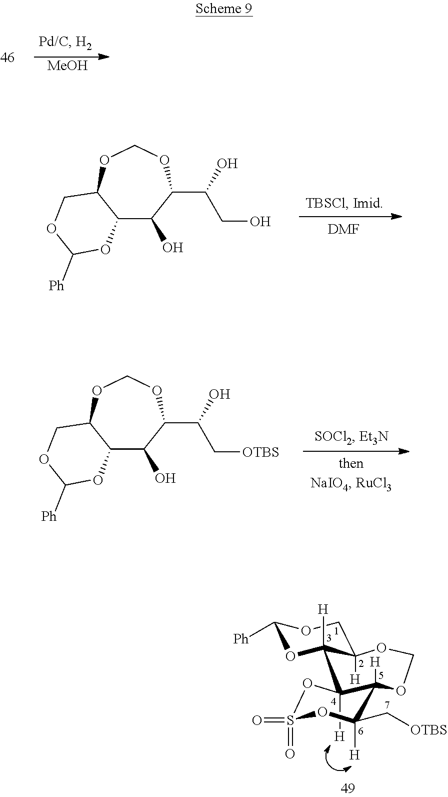

In order to obtain the other diastereomer, with the S configuration (at C-2), the olefin 45 was treated with AD-mix-β in tert-BuOH—H₂O (1:1). A diastereomeric ratio of 7:1 was obtained, with the major isomer 50 in 64% yield. The diol 50 was converted into the corresponding cyclic sulfate 52, as for the case of 48 (Scheme 10).

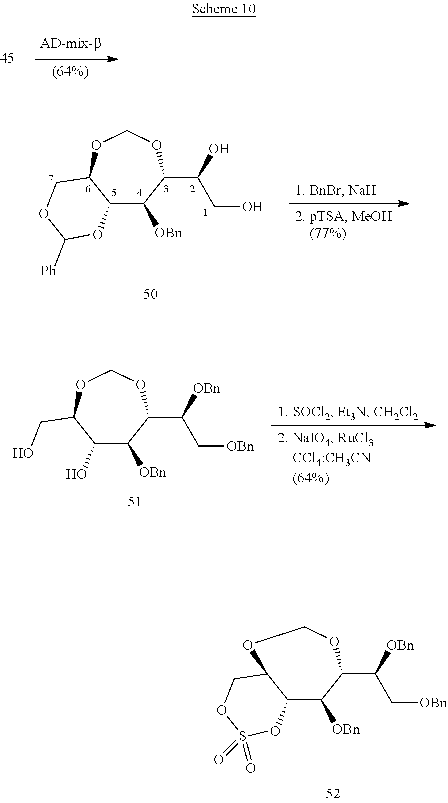

With the cyclic sulfates 48 and 52 in hand, the inventors turned their attention to the coupling reactions with the thio-arabinitol 53³⁷ in 1,1,1,3,3,3-hexafluoro-2-propanol (HFIP) as a solvent containing K₂CO₃. The coupling reaction was found to proceed slowly at 75° C., with some decomposition occurring above 80° C., so the reactions were terminated after stirring at 75° C. for 7 days to give the corresponding coupled products 54 and 57 in 67% and 61% yield, respectively (Scheme 11). Finally, deprotection of 54 was carried out by treatment with 90% trifluoroacetic acid in water. The methylene groups were found to survive these reaction conditions, as well as treatment with 5% aqueous hydrochloric acid at 40° C., and yielded compound 55.

Treatment of 55 with 1.0 M BCl₃ in methylene chloride was successful in removing all protecting groups, but also resulted in desulfonation, thus leading to compound 56. Similarly, the protected compound 57 was also treated with 1.0 M BCl₃ to yield compound 58. Naturally occurring de-O-sulfonated kotalanol 5 has been obtained from kotalanol by Yoshikawa et al.,¹¹ and has also been isolated recently by Ozaki et al.,¹⁴ as claimed by Yoshikawa et al.¹² With the de-O-sulfonated compounds 56 and 58 in hand, it was therefore possible to compare their physical data to those of authentic de-O-sulfonated kotalanol 5.

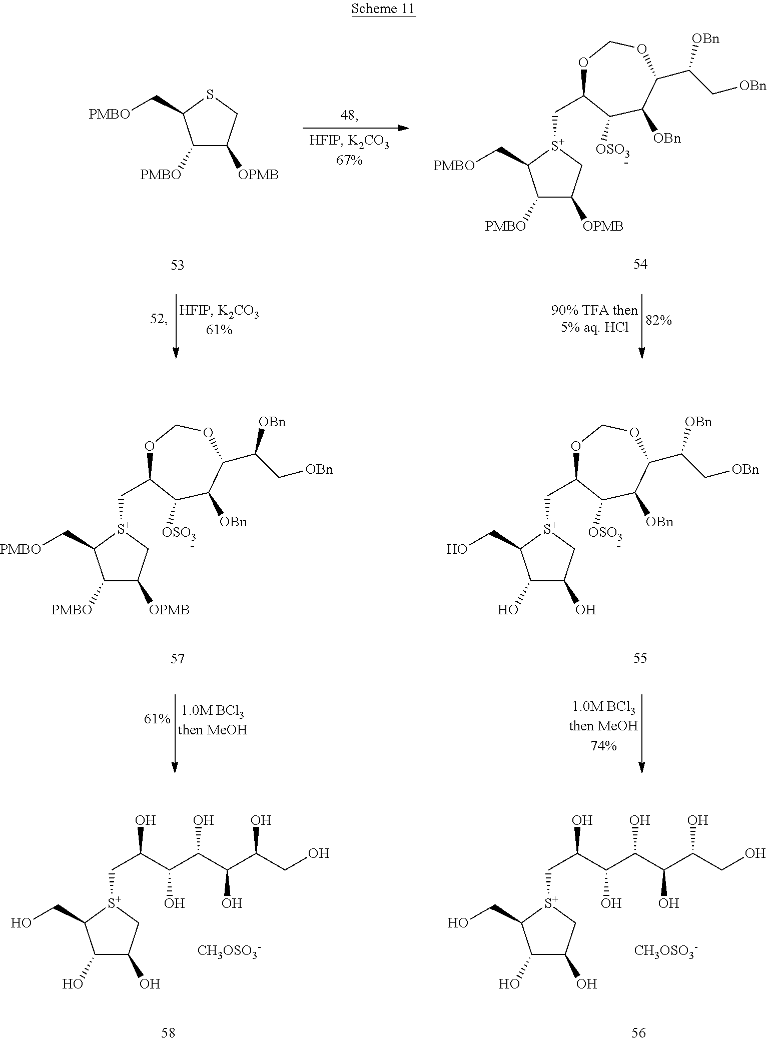

### 3.0 CHARACTERIZATION OF STEREOISOMERS AND ANALOGUES OF KOTALANOL

Compound 17 was fully characterized by spectroscopic methods. The proton and carbon signals in the ¹H and ¹³C NMR spectra of 17 in D₂O were assigned unambiguously with the aid of ¹H—¹H COSY, HMQC, and HMBC experiments. The stereochemistry at the stereogenic sulfonium-ion center was assigned by means of a NOESY experiment which showed an H-5 to H-1′ correlation, implying that isomer 17 has an anti relationship between C-5 and C-1′. MALDI-TOF mass spectrometry in the positive mode showed base peaks for masses attributable to M+Na and lower intensity peaks corresponding to M+H and M+H—SO₃H. The compounds were also characterized by high-resolution mass spectrometry and compound 17 exhibited a dimer cluster-ion peak at lower intensity.

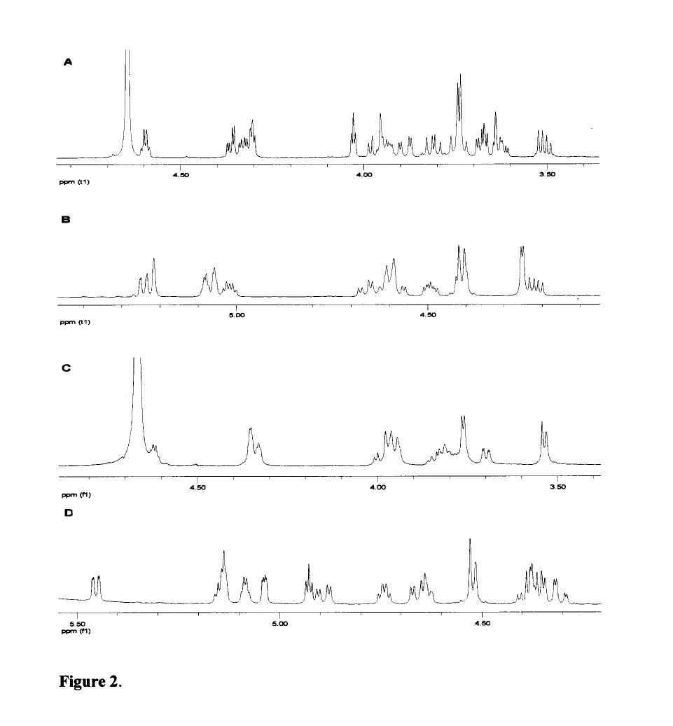

NMR analysis of 17 and 18 was carried out both in D₂O and pyridine-d₅ solution (FIG. 2). These studies revealed that the ¹H NMR spectra in pyridine-d₅ gave downfield shifts compared to those in D₂O, together with differential spectral patterns. A careful comparison indicated unusual downfield shifts (most downfield resonances) for H-2, H-3 and H-2′ in D₂O. This trend might be explained by the greater solvation of the ion pair in the more polar solvent D₂O that induces a greater partial positive charge and a resultant deshielding of H-2, H-3 and H-2′. In contrast to these observations, our NMR studies in pyridine-d₅ showed the most downfield resonances (δ 5.34 for 17 and δ 5.47 for 18) that were assigned to H-3′ using 2D-NMR techniques including TOCSY and HMBC.

TABLE 3
Comparison of ¹³C NMR data^{a} and discrepancies^{b} of the chemical shifts
of compounds 17 and 18 relative to those reported for kotalanol 4.

| Position | 17 | Kotalanol 4 | 18 |
|---|---|---|---|
| 1′ | 53.4 (−0.3) | 53.7 | 53.3 (−0.4) |
| 2′ | 68.0 (−1.4) | 67.4 | 68.3 (+0.9) |
| 3′ | 81.8 (+3.9) | 77.9 | 80.5 (+2.5) |
| 4′ | 68.1 (−2.4) | 70.5 | 70.4 (−0.1) |
| 5′ | 72.8 (+1.5) | 71.3 | 73.5 (+1.8) |
| 6′ | 75.5 (+3.0) | 72.5 | 73.9 (+1.4) |
| 7′ | 65.6 (+0.2) | 65.3 | 64.6 (−0.7) |
| 1 | 50.3 (+0.1) | 50.2 | 50.5 (+0.3) |
| 2 | 78.4 (+0.3) | 78.1 | 78.4 (+0.3) |
| 3 | 79.3 (−0.1) | 79.4 | 79.3 (−0.1) |
| 4 | 72.5 (+0.3) | 72.2 | 72.5 (+0.3) |
| 5 | 60.0 (0) | 60.0 | 60.2 (+0.2) |

^{a}in pyridine-d₅,
^{b}values in bold.

TABLE 4
Comparison of ¹H NMR data^{a} and discrepancies^{b} of the chemical shifts
of compounds 17, 18 relative to those reported for kotalanol 4.

| Position | 17 | Kotalanol 4 | 18 |
|---|---|---|---|
| 1′ | 4.84 (dd), 4.66 (dd) | 4.93 (dd), 4.65 (dd) | 4.91 (dd), 4.67 (dd) |
| 2′ | 5.09 (m) | 5.24 (m) | 5.16 (ddd) |
| 3′ | 5.34 (d) | 5.64 (dd) | 5.47 (m) |
| 4′ | 5.31 (br s) | 5.12 (br s) | 5.05 (dd) |
| 5′ | 4.65 (m) | 5.86 (dd-like) | 4.94 (dd) |
| 6′ | 4.57 (m) | 4.88 (ddd-like) | 4.76 (ddd) |
| 7′ | 4.49 (dd), 4.31 (dd) | 4.50 (dd), 4.25 (dd) | 4.41 (dd), 4.39 (dd) |
| 1 | 4.30 (d) | 4.31 (br s) | 4.36 (dd), 4.32 (dd) |
| 2 | 5.09 (m) | 5.08 (dd-like) | 5.09 (m) |
| 3 | 5.16 (br s) | 5.16 (br s) | 5.16 (br s) |
| 4 | 4.65 (m) | 4.64 (t-like) | 4.65 (m) |
| 5 | 4.54 (d) | 4.54 (dd-like) | 4.55 (d) |

^{a}in pyridine-d₅,
^{b}values in bold.

Physical data of compounds 17 and 18 were compared with those reported for kotalanol 4.¹³ The specific rotation and melting point of 18 ([α]_{D}²⁵+12.0 (c 0.1, MeOH) and mp 169-171° C., respectively) were found to be in agreement with the reported values ([α]_{D}²⁷+11.5 (MeOH); mp 175-177° C.) for kotalanol 4. The optical rotation and melting point of 17 were found to be [α]_{D}²⁵+16.0 (c 0.1, MeOH) and mp 164-166° C., respectively. Comparison of the ¹H and ¹³C NMR spectroscopic data of 18 with those reported¹³ for kotalanol 4 revealed that the sets of data in pyridine-d₅ are not identical. A careful check of ¹H NMR data of kotalanol 4 and compound 18 indicated that there was a difference in chemical shifts (±δ0−0.17) (Table 4). However, the most notable difference was the chemical shift of H-5′, reported at δ 5.86 ppm in kotalanol 4. In contrast, no signal below δ 5.47 ppm was observed in the spectrum of compound 18. The H-5′ signals of 17 and 18 appeared at δ 4.65 and δ 4.91, respectively. The ¹³C NMR data also reveal discrepancies between those of 18 and those reported for kotalanol 4, especially for C-3′; specifically, C-3′ is shielded in kotalanol. Comparison of accumulated data to date for related analogues indicates that C-3′ exhibits an upfield shift when the sulfate moiety at C-3′ and the hydroxyl group at C-5′ are anti to one another. Thus, in kotalanol 4, C-3′ resonates at 77.9 ppm; the corresponding shifts in 8, 12, and 14 are 78.9,¹⁸ 77.62^{20b} and 78.3 ppm,²¹ respectively. This shielding can be attributed to the γ-gauche effect of the axially-oriented hydroxyl group (Chart 6) acting on C-3′. The proximity of the negatively-charged sulfate moiety to H-5′ would also account for the unusual deshielding of this hydrogen. This suggests that kotalanol 4 has the opposite configuration at C-5′ to 17 and 18, with an anti relationship between the substituents at C-3′ and C-5′. This still left the configuration at C-6′ unspecified. The configuration at C-6′ was confirmed via the structures of compounds 19 and 20, as discussed below.

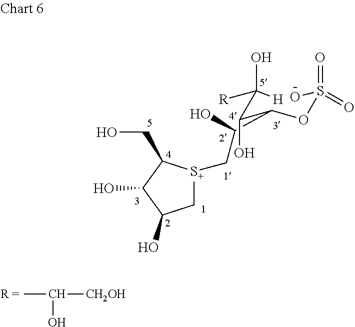

Determination of the stereochemistry of kotalanol 4 at C-6′ was confirmed by characterization of the de-O-sulfonated compounds 56 and 58. Comparison of the ¹H and ¹³C NMR data for these compounds with those of the naturally-derived de-O-sulfonated kotalanol 5 is shown in Table 5. The synthetic compounds 56 and 58 have CH₃OSO₃⁻ as the external counter-ion, as confirmed by ¹H and ¹³C NMR spectroscopy. In addition, Yoshikawa et al. have reported that the counter-ion has no significant effect on the NMR chemical shifts.¹² The ¹H and ¹³C NMR spectra of 56 and 58 were recorded in CD₃OD and assigned unambiguously with the aid of ¹H—¹H COSY, HMQC, HMBC and APT experiments. The stereochemistry at the stereogenic sulfonium center in 56 and 58 was established by means of NOESY experiments. A correlation between H-1′ and H-4, confirmed the trans relationship between the side chain and the C-4 substituent of the thio-arabinitol moiety, as found in the inventors' previous studies.¹⁷ These data show that naturally occurring de-O-sulfonated kotalanol 5 possesses the structure displayed by 58. This conclusion is corroborated by the optical rotation data (56 ([α]_{D}²³−4.0 (c 0.8, MeOH)); 58 ([α]_{D}²³+10.0 (c 0.6, MeOH)); naturally derived de-O-sulfonated kotalanol 5 ([α]_{D}²³+13.0 (c 0.6, MeOH)).

TABLE 5
Comparison of ¹H and ¹³C NMR data to those reported
for de-O-sulfonated kotalanol in CD₃OD

| | ¹H NMR data | | | ¹³C NMR data | | |
|---|---|---|---|---|---|---|
| | 56 | 5³ | 58 | 56 | 5³ | 58 |
| 1′ | 3.94, 3.75 | 3.94, 3.76 | 3.94, 3.76 | 52.7 | 52.7 | 52.7 |
| 2′ | 4.17 | 4.18 | 4.18 | 69.4 | 69.7 | 69.7 |
| 3′ | 3.85 | 3.84 | 3.85 | 74.0 | 70.2 | 70.2 |
| 4′ | 3.88 | 3.65 | 3.65 | 71.9 | 71.3 | 71.2 |
| 5′ | 3.71 | 3.85 | 3.84 | 73.1 | 73.6 | 73.6 |
| 6′ | 3.83 | 3.93 | 3.93 | 74.8 | 71.7 | 71.7 |
| 7′ | 3.80, 3.67 | 3.66 | 3.66 | 64.4 | 65.0 | 64.9 |
| 1 | 3.86 | 3.87 | 3.87 | 51.9 | 51.9 | 51.9 |
| 2 | 4.62 | 4.62 | 4.62 | 79.4 | 79.4 | 79.4 |
| 3 | 4.37 | 4.37 | 4.37 | 79.5 | 79.5 | 79.5 |
| 4 | 4.02 | 4.02 | 4.01 | 73.7 | 73.7 | 73.7 |
| 5 | 4.05, 3.93 | 4.05, 3.93 | 4.05, 3.93 | 61.1 | 61.1 | 61.1 |

The above results constitute, therefore, a formal structure proof of kotalanol 4, 20, the naturally occurring glycosidase inhibitor from Salacia reticulata (Scheme 12). These results also confirm that the heptitol side chain of kotalanol is 5-O-sulfonyl-D-perseitol, a naturally occurring heptitol isolated from fruits,^{38a} leaves.^{38b} and the wound exudate^{38c} of avocado trees. Further corroboration was obtained by comparison of the physical data of the heptitol, obtained upon treatment of intermediate 50 with 1.0 M BCl₃ (Scheme 12), with those of commercially available D-perseitol.

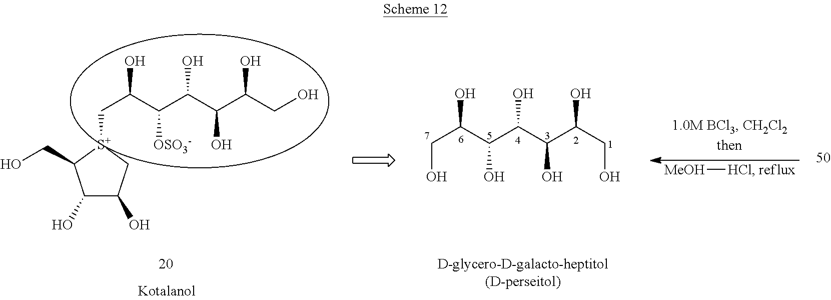

### 4.0 DIRECT SYNTHESIS OF KOTALANOL

Having confirmed the structure of kotalanol, the inventors developed a method for its synthesis from a cyclic sulfate derived from D-perseitol. D-perseitol was first converted into 1.3:5,7-di-O-benzylidene-D-perseitol 59³⁹ and the secondary hydroxyl groups were then protected as PMB ethers. The protected compound was treated with a catalytic amount of PTSA in methanol to yield the regioisomeric diols, 60 and 61, in 44 and 34% yield, respectively. These isomers were conveniently separated by column chromatography and differentiated by careful NMR analysis as shown in Scheme 13 and Table 6. Thus, the compound with lower coupling constant values for H-2 (J=1.2, 1.2, 1.2 Hz) and showing HMBC correlations between the benzylidene acetal carbon and C-1 and C-3, was identified as being the desired diol 61, to be taken on to the next step. The compound with higher coupling constants for H-6 (J=4.8, 10.8, 9.6 Hz), and showing HMBC correlations between the benzylidene acetal carbon and C-5 and C-7 was identified as being the undesired diol 60.

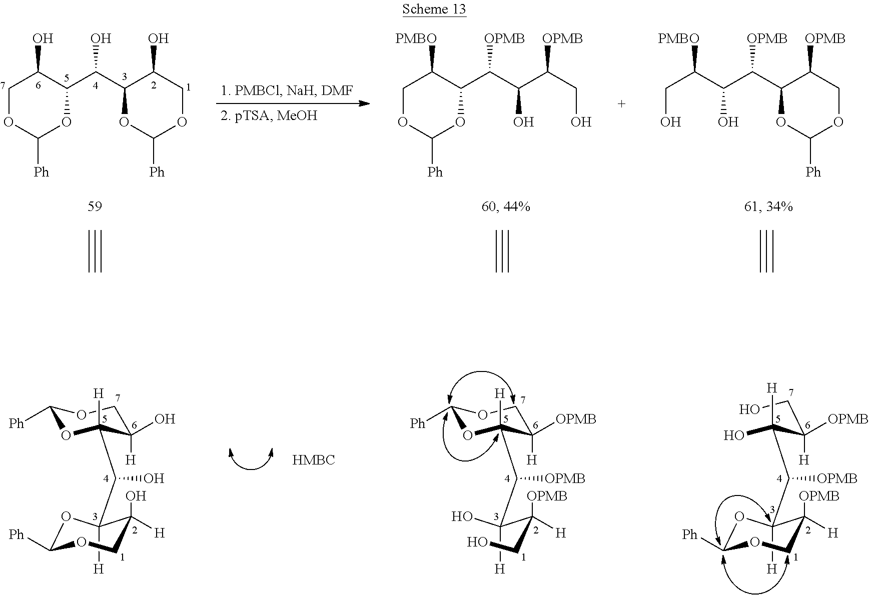

TABLE 6
Comparison of the observed coupling constants for H-2
and H-6 in the ¹H NMR spectra of compounds 59-62.

Coupling constant^{d} (Hz)

| Compound | H-2 (J₁ₐ₋₂, J_{1b-2}, J₂₋₃) | H-6 (J₇ₐ₋₆, J_{7b-6}, J₅₋₆) |
|---|---|---|
| 59^{a} | 1.2, 1.2, 1.2 | 5.4, 10.2, 9.6 |
| 60^{b} | 4.2, 4.2, 1.8 | 4.8, 10.8, 9.6 |
| 61^{c} | 1.2, 1.2, 1.2 | 4.2, 4.2, 8.4 |
| 62^{c} | 1.2, 1.2, 1.8 | 7.2, 7.2, 9.6 |

^{a}Solvent: pyridine-d₅ + CD₃OD;
^{b}Solvent: CDCl₃ + D₂O;
^{c}Solvent: CDCl₃,
^{d}600 MHz NMR.

The desired diol 61 was first converted into the cyclic sulfate 62 and then coupled with the PMB-protected thio-D-arabinitol 53 as before to yield compound 63 in 69% yield. The PMB and benzylidene protecting groups were removed in one pot by treatment with 80% trifluoroacetic acid (TFAA) in water at room temperature to yield compound 20 in 93% yield (Scheme 14).

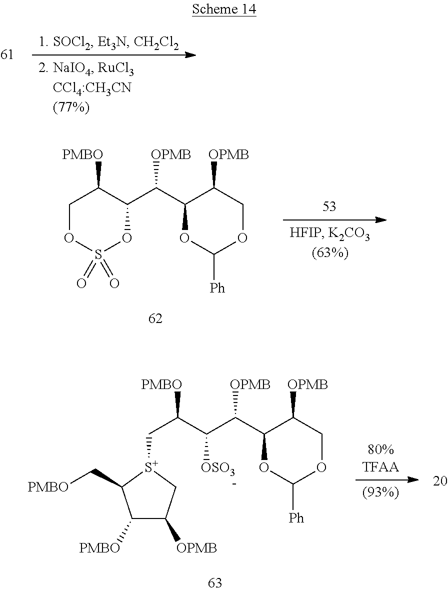

Detailed 1D and 2D NMR experiments of compound 20 in pyridine-d₅ were performed and the data were compared with those reported for kotalanol 4.¹³ The choice of pyridine-d₅ as solvent caused broad peaks due to coupling with the hydroxyl groups and hence a D₂O exchange experiment was necessary to calculate the exact coupling constants. The stereochemistry at the stereogenic sulfur atom was established by means of NOESY experiments in analogous manner to those performed for the de-O-sulfonated compounds 56 and 58. The ¹H NMR data of compound 20 in pyridine-d₅ compare favorably with those reported for kotalanol (with deviations of ±0-0.1 ppm), except for the chemical shift of H-5′ (Table 7). The reported chemical shift value for H-5′ was at 5.86 ppm,¹³ whereas the ¹H NMR spectrum of compound 20 showed the corresponding signal at 4.86 ppm (confirmed with the aid of ¹H—¹H COSY, HMQC and HMBC experiments). In fact, compound 20 had no signal appearing below 5.64 ppm. However, all of the ¹³C NMR chemical shifts of compound 20 correlate well with those reported¹³ for kotalanol, with deviations of ±0-0.1 ppm (Table 7). This mismatch of ¹H NMR chemical shift values of H-5′ was also one of the major discrepancies noted in the inventors' previously synthesized kotalanol analogues.⁴⁰ To eliminate this discrepancy unambiguously, the inventors subjected compound 20 to de-O-sulfonation using the reported procedure,¹¹ and compared the ¹H and ¹³C NMR data (in CD₃OD) of the resulting de-O-sulfonated compound with those reported for de-O-sulfonated kotalanol.¹¹ These results indicated that, indeed, all ¹H and ¹³C NMR chemical shift values agreed with those reported.¹¹ Hence, it is reasonable to conclude that the reported¹³ chemical shift value for H-5′ in kotalanol must be in error. The inventors note also that the optical rotation of compound 20 ([α]_{D}²³−5.7° (c 0.7, MeOH)) is not in agreement with the reported value ([α]_{D}²⁷+11.5° (MeOH)); the inventors obtained a specific rotation of +7.0° for 20 in water (c 0.6, H₂O). To confirm the change in sign of optical rotation as a function of solvent, the optical rotation of the same sample was repeated in MeOH and in H₂O, alternately. Once again, in methanol, compound 20 showed levo (−) rotation and in water, showed dextro (+) rotation. The inventors attribute this discrepancy to the solubility difference of compound 20 in MeOH and H₂O. It is also noted that the optical rotations of the recent analogues of kotalanol, made by Muraoka et al,^{23b} were also reported in H₂O, and that the reported data for kotalanol¹³ do not indicate the concentration at which the optical rotation was measured. Hence, the inventors surmise that the solvent reported¹³ for the measurement of the optical rotation was also in error.

Based on the successful conversion of synthetic material 20 to de-O-sulfonated kotalanol and comparison of physical data with those of kotalanol (given the prior art errors noted above), the inventors concluded that the absolute stereostructure of kotalanol 4 is the structure displayed by 20, bearing the D-perseitol configuration in the acyclic side chain.

TABLE 7
Comparison of ¹H and ¹³C NMR data with those
reported for kotalanol in pyridine-d₅.

| | |
|---|---|
| ¹H NMR data | ¹³C NMR data |

| | 20 | Kotalanol | 20 | Kotalanol |
|---|---|---|---|---|
| 1′ | 4.65, 4.93 | 4.65, 4.93 | 53.8 | 53.7 |
| 2′ | 5.24 | 5.24 | 67.4 | 67.4 |
| 3′ | 5.64 | 5.64 | 77.9 | 77.9 |
| 4′ | 5.12 | 5.12 | 70.5 | 70.5 |
| 5′ | 4.86 | 5.86 | 71.3 | 71.3 |
| 6′ | 4.88 | 4.88 | 72.6 | 72.5 |
| 7′ | 4.24, 4.40 | 4.25, 4.50 | 65.4 | 65.3 |
| 1 | 4.31 | 4.31 | 50.1 | 50.2 |
| 2 | 5.07 | 5.08 | 78.1 | 78.1 |
| 3 | 5.15 | 5.16 | 79.4 | 79.4 |
| 4 | 4.62 | 4.64 | 72.2 | 72.2 |
| 5 | 4.51 | 4.51 | 60.0 | 60.0 |

Derivatives of D-perseitol with other protecting groups could likewise be used in the synthesis of analogues of kotalanol or de-O-sulfonated kotalanol. For example, the synthesis might involve the direct displacement of a primary halide or sulfonate ester of perseitol (suitably protected at the other hydroxyl groups) by the protected thioarabinitol, as shown below in Scheme 15 for the synthesis of de-O-sulfonated kotalanol and its analogues from D-perseitol.

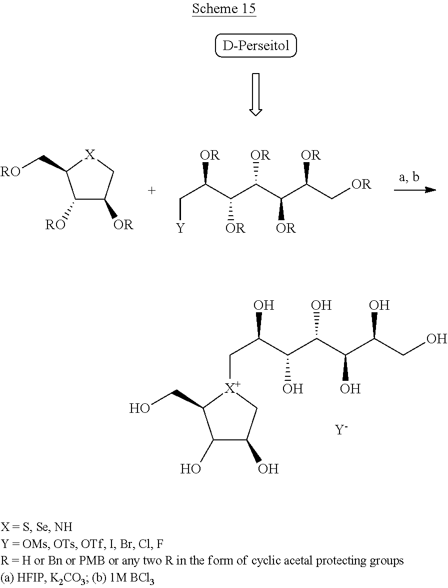

Similarly, for example, different stereoisomers of de-O-sulfonated analogues of kotalanol may be synthesized directly from appropriate protected heptitols (for example, the primary halide or sulfonate ester), as shown in Scheme 16.

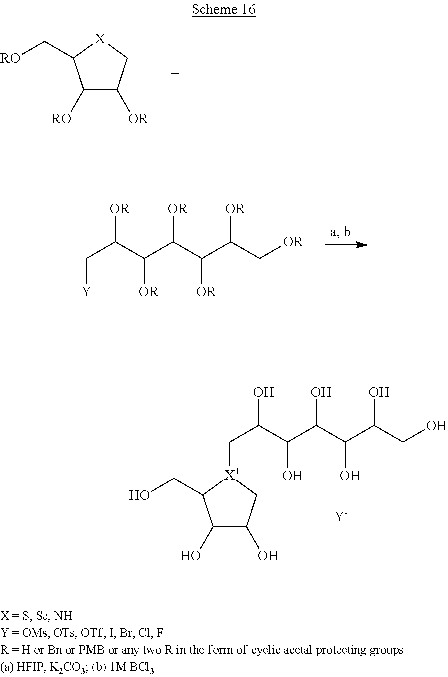

### 5.0 SYNTHESIS OF SELENIUM AND NITROGEN ANALOGUES OF KOTALANOL AND THEIR STEREOISOMERS

Following the synthetic route of kotalanol 20, the inventors have also prepared the corresponding selenium and nitrogen analogues of kotalanol, 67 and 72, using the PMB-protected 1,4-anhydro-4-seleno-D-arabinitol 64 and 1,4-dideoxy-1,4-imino-D-arabinitol 71, respectively, as shown in Schemes 17 and 18. In the coupling reaction of the selenoarabinitol 64 with the cyclic sulfate 62, in addition to the desired coupled product 65, a diastereomer 66, with respect to the stereogenic selenium center, was also obtained. Deprotection yielded compounds 67 and 68.

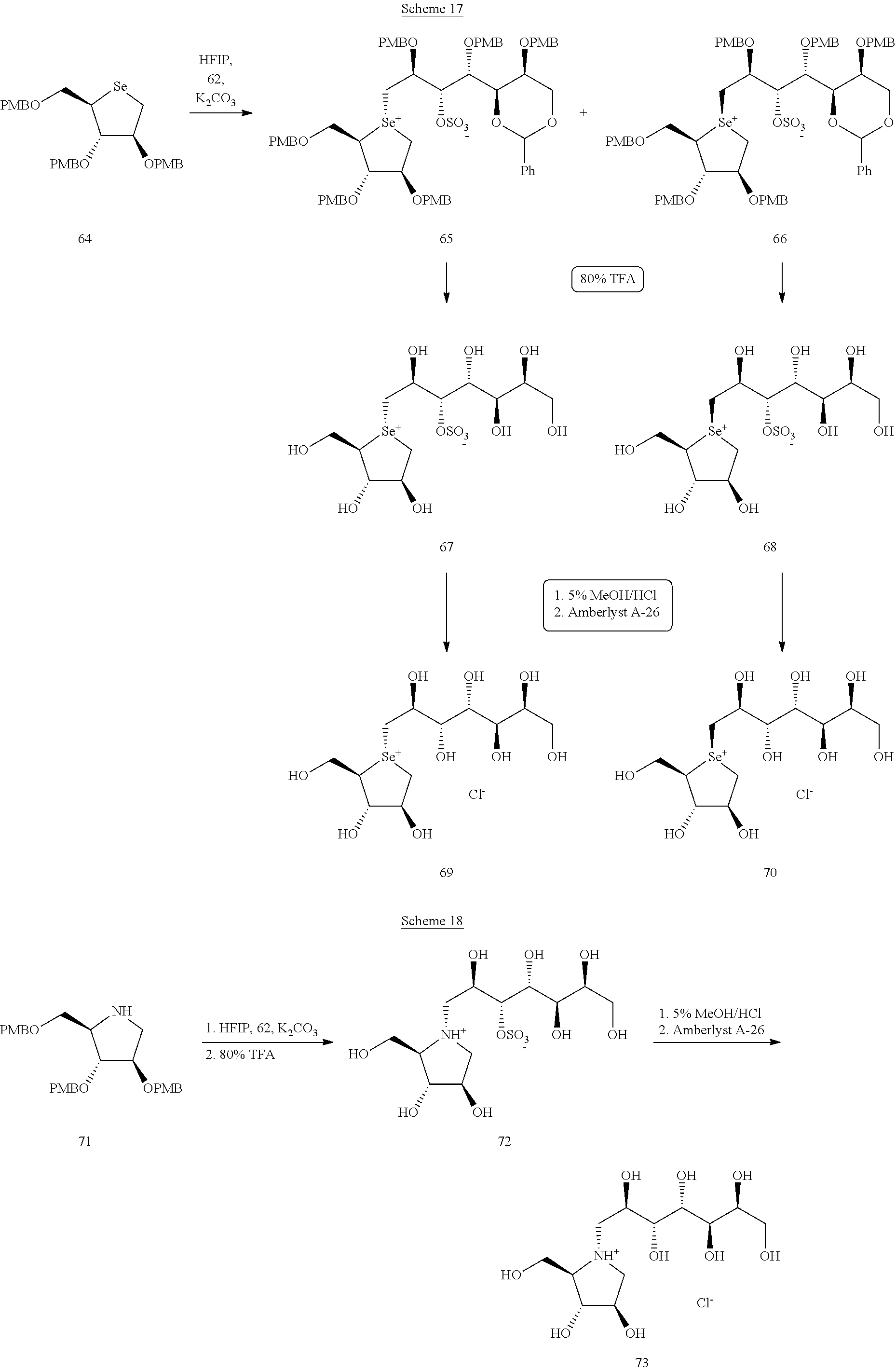

### 6.0 SYNTHESIS OF DE-O-SULFONATED DERIVATIVES OF KOTALANOL, ITS ANALOGUES, AND STEREOISOMERS THEREOF

The synthesis of de-O-sulfonated kotalanol 58 is described above with reference to Schemes 7, 10 and 11, while synthesis of its stereoisomer 56 is described above with reference to Schemes 7, 8 and 11.

Compounds 67, 68 and 72 were also converted into their corresponding de-O-sulfonated derivatives, 69, 70 and 73, respectively, using 5% methanolic HCl (Schemes 17 and 18).

Similarly, compounds 17 and 18 were also converted into their corresponding de-O-sulfonated derivatives, 74 and 75, respectively, using 5% methanolic HCl (Scheme 19).

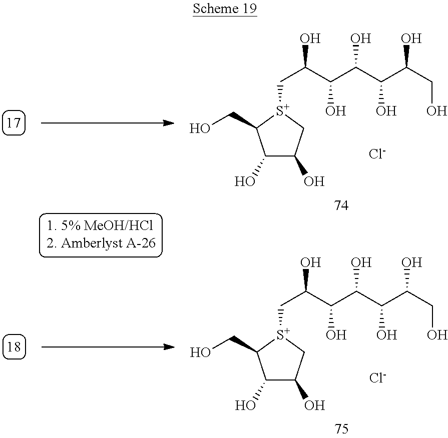

### 7.0 EVALUATION OF THE BIOLOGICAL ACTIVITY OF KOTALANOL, ITS ANALOGUES AND STEREOISOMERS THEREOF

The inhibitory activities of some of the compounds synthesized by the inventors against recombinant human maltase glucoamylase (MGA), a critical intestinal glucosidase involved in the processing of oligosaccharides of glucose into glucose itself, were tested. These activities were evaluated as described in the inventors' previous studies, which are incorporated by reference herein.^{21,22,40} The seven carbon-chain analogues of salacinol, 17 and 18 inhibited MGA with Ki values of 0.13±0.02 and 0.1±0.02 μM, respectively. The observed inhibition data is consistent with the structure activity relationships established previously for the lower homologues (Table 1).

The tested compounds appear to be potent inhibitors of MGA (Table 8).⁴¹ Both the de-O-sulfonated compounds, 56 and 58, inhibited MGA with IC₅₀ values of 80 and 50 nM, respectively, whereas synthetic kotalanol 20 inhibited MGA with an IC₅₀ value of 300 nM. Thus, de-O-sulfonation appears to be beneficial, and results in a six-fold increase in the inhibitory activity of compound 58 when compared to synthetic kotalanol 20. The inventors note that 56 and 58 constitute the most active in the class of zwitterionic glycosidase inhibitors that are related to salacinol and kotalanol, to date, while 17 and 18 constitute the most active chain-extended analogues of salacinol to date.

TABLE 8
Inhibitory activities of compounds 56, 58 and 20 against MGA.

| Compound | IC₅₀ (nM) |
|---|---|
| 56 | 80 |
| 58 | 50 |
| 20 | 300 |

### 8.0 USE OF SYNTHETIC KOTALANOL, ITS ANALOGUES AND STEREOISOMERS THEREOF

The synthetic compounds discussed in this application may be used, for example, as a standard to calibrate or grade natural herbal remedies containing kotalanol, de-O-sulfonated kotalanol, or another naturally occurring analogue or stereoisomer of kotalanol. For example, a known quantity of kotalanol 20 may be synthesized as described above, and the known characteristics of synthetic kotalanol 20 may be compared to a sample of an extract that is proposed to be used or sold as a natural or herbal remedy for disorders that may be treated by glycosidase inhibitors, for example diabetes. Suitable means of comparison for which a known quantity of kotalanol 20 may be used as a standard to calibrate a natural herbal remedy include, for example, HPLC, capillary electrophoresis, NMR. HPLC-mass spectrometry, or other analytical techniques known to those skilled in the art.

The synthetic compounds discussed in this application may also be used themselves, optionally in combination with a pharmaceutically acceptable carrier, as a treatment for disorders in which glycosidase inhibitors are effective to treat the disorder, such as, for example, diabetes. The glycosidases to be inhibited may include intestinal glucosidases, such as, for example, maltose glucoamylase (MGA).

### 9.0 EXAMPLES

The invention is further described with reference to the following specific examples, which are not meant to limit the invention, but rather to further illustrate it.

### 9.1 Experimental Methods

General—Compounds 17, 18, 21-38: Optical rotations were measured at 23° C. ¹H and ¹³C NMR spectra were recorded at 500 and 125 MHz, respectively. All assignments were confirmed with the aid of two dimensional experiments (¹H—¹H COSY, HMQC and HMBC). Column chromatography was performed with Merck silica gel 60 (230-400 mesh). MALDI-TOF mass spectra were recorded on a perSeptive Biosystems Voyager-DE spectrometer, using 2.5-dihydroxybenzoic acid as a matrix.

General—Compounds 19, 20, 39-75: Optical rotations were measured at 23° C. ¹H and ¹³C NMR spectra were recorded at 600 and 150 MHz, respectively. All assignments were confirmed with the aid of two-dimensional ¹H, ¹H (COSYDFTP) or ¹H, ¹³C (INVBTP) experiments using standard pulse programs. Column chromatography was performed with Silica gel 60 (230-400 mesh). High resolution mass spectra were obtained by the electrospray ionization method, using an Agilent 6210 TOF LC/MS high resolution magnetic sector mass spectrometer.

Enzyme Kinetics: Kinetic parameters of MGA with compounds 17 and 18 were determined using the pNP-glucose assay to follow the production of p-nitrophenol upon addition of enzyme (500 nM). The assays were carried out in 96-well microtiter plates containing 100 mM MES buffer pH 6.5, inhibitor (at 3 different concentrations), and p-nitrophenyl-D-glucopyranoside (pNP-glucose, Sigma) as substrate (2.5, 3.5, 5, 7.5, 15 and 30 mM) with a final volume of 50 μL. Reactions were incubated at 37° C. for 35 min and terminated by addition of 50 μl of 0.5 M sodium carbonate. The absorbance of the reaction product was measured at 405 nm in a microtiter plate reader. All reactions were performed in triplicate and absorbance measurements were averaged to give a final result. Reactions were linear within this time frame. The program GraFit 4.0.14 was used to fit the data to the Michaelis-Menten equation and estimate the kinetic parameters, Km, K_{mobs} (Kₘ in the presence of inhibitor) and Vₘₐₓ, of the enzyme. Kᵢ values for each inhibitor were determined by the equation Kᵢ=[I]/((K_{mobs}/Kₘ) D1). The Kᵢ reported for each inhibitor was determined by averaging the Kᵢ values obtained from three different inhibitor concentrations.

### 9.2 Synthesis of Compounds 17 and 18

### 9.2.1 Preparation of Cyclic Sulfates 22 and 23

Allyl 4,6-O-benzylidene-2,3-O-(2′,3′-dimethoxybutane-2′,3′-diyl)-α,β-D-glucopyranoside (24)—To a suspension of D-glucose (30 g, 0.16 mol) in allyl alcohol (100 mL) was added AcCl (1 mL) and the reaction mixture was refluxed for 12 h. The reaction mixture was cooled to room temperature (“rt”) and the reaction was quenched by addition of excess triethylamine (5 mL). The solvent was removed under reduced pressure and dried on high-vacuum for 12 h. To the residue in dry MeOH (200 mL), 2,3-butanedione (17.2 mL, 0.2 mol), trimethyl orthoformate (70 mL, 0.6 mol), and CSA (500 mg) were added and the reaction mixture was refluxed for 24 h. When TLC analysis of aliquots (Hexanes:EtOAc, 1:1) showed total consumption of the starting material, the reaction mixture was cooled to room temperature and excess triethylamine (4 mL) was added. The solvents were evaporated; the residue was dissolved in EtOAc (200 mL), and washed with brine and dried over Na₂SO₄, then concentrated to give brownish oil. The latter was dissolved in DMF (100 mL) and dimethoxybenzaldehyde (30 g, 0.16 mol), and p-toluenesulfonic acid (300 mg) were added. The reaction mixture was stirred at 60° C. on a rotary evaporator under vacuum for 2 h. The reaction was then quenched by adding triethylamine, the solvent removed, and the residue was dissolved in EtOAc (150 mL), washed with saturated aqueous NaCl (50 mL), dried over Na₂SO₄, and concentrated to give brown syrup. Purification by column chromatography on silica gel (Hexane:EtOAc, 1:1) yielded compound 24 as a white solid (22 g, 31%). Data for the β-isomer: ¹H-NMR (CDCl₃): δ 7.36-7.26 (Ar), 5.93 (1H, dddd, allyl), 5.53 (1H, s, Ph-CH), 5.35 (1H, d, allyl), 5.19 (1H, d, allyl), 4.64 (1H, d, J_{1,2}=7.8 Hz, H-1), 4.36 (1H, dd, allyl), 4.30 (1H, dd, J_{6a,6b}=10.4, J_{6a,5}=4.8 Hz, H-6a), 4.16 (1H, dd, 3.99 (1H, dd, J_{3,2}=9.6 Hz, H-3), 3.82 (1H, dd, J_{6b,5}=10.2 Hz, H-6b), 3.72 (1H, dd, J_{4,5}=9.0 Hz, H-4), 3.69 (1H, dd, H-2), 3.45 (1H, ddd, H-5), 3.30, 3.28 (2×-OMe), 1.33, 1.33 (2×-Me). ¹³C NMR: δ 137.4-117.2 (Ar, allyl), 101.4 (Ph-CH), 100.6 (C-1), 99.9, 99.6 (BDA), 78.0 (C-4), 70.5 (C-2, allyl), 69.7 (C-3), 68.9 (C-6), 67.6 (C-5), 48.2, 48.1 (2×-OMe), 17.8, 17.8 (2×-Me).

4,6-O-Benzylidene-2,3-O-(2′,3′-dimethoxybutane-2′,3′-diyl)-α,β-D-glucopyranose (25)—t-BuOK (0.07 mol, 7.8 g) was added to a solution of allyl-5,7-O-benzylidene-3,4-O-(2′,3′-dimethoxybutane-2′,3′-diyl)-α,β-D-glucopyranoside (24) (16.2 g, 0.038 mol) in DMF (200 mL), and the mixture was stirred for 2 h at 80° C. The reaction mixture was cooled to rt and extracted with EtOAc (3×150 mL). The organic layer was washed with 1M aqueous HCl and dried over Na₂SO₄. The solvent was removed under reduced pressure to give the enol ether as a brown syrup. The residue was redissolved in a mixture of THF and water (4:1, 150 mL) and treated with iodine (0.07 mol) for 1.5 h. The reaction was then quenched by addition of a saturated solution of Na₂S₂O₃. The organic layer was separated and the aqueous layer was extracted with EtOAc (2×50 mL). The combined organic layers were washed with brine solution, dried over Na₂SO₄, and concentrated. The residue was purified by column chromatography to give 25 as a white amorphous solid (12.2 g, 84%). Data for the β-isomer: ¹H-NMR (CDCl₃): δ 7.53-7.35 (5H, Ar), 5.54 (1H, Ph-CH), 5.29 (1H, dd, J_{1,2}=3.5, J_{1,—OH}=3.0 Hz, H-1), 4.70 (1H, dd, J_{3,2}=10.8, J_{3,4}=9.6 Hz, H-3), 4.22 (1H, dd, J_{6a,6b}=10.2, J_{6a,5}=4.8 Hz, H-6a), 4.17 (1H, dd, H-2), 4.06 (1H, ddd, J_{5,4}=9.4, J_{5,6b}=10.4, H-5), 3.76 (1H, dd, H-6b), 3.54 (1H, dd, H-4), 3.42, 3.39 (2×-OMe), 3.01 (1H, br s, —OH), 1.39 (2×-Me). ¹³C NMR: δ 137.4-126.7 (Ar), 102.2 (Ph-CH), 101.8, 101.7 (BDA), 92.4 (C-1), 81.1 (C-4), 72.0 (C-2), 69.1 (C-3), 68.9 (C-6), 63.4 (C-5), 48.5, 48.4 (2×-OMe), 19.1, 19.1 (2×Me). Anal. Calcd. for C₁₉H₂₆O₈: C, 59.68; H, 6.85. Found: C, 59.82; H, 6.49.

5,7-O-Benzylidene-3,4-O-(2′,3′-dimethoxybutane-2′,3′-diyl)-D-glueo-hept-1-enitol (26)—n-BuLi (n-hexane solution, 0.058 mol, 2.90 equiv) was added dropwise to a solution of methyltriphenylphosphonium bromide (21.4, 0.06 mmol, 3.0 equiv) in dry THF (80 mL) at −78° C. under N₂. The mixture was stirred for 1 h at the same temperature. A solution of 25 (7.8 g, 0.02 mol) in dry THF (10 mL) was introduced into the solution at −78° C., and the resulting solution was stirred for an additional 30 min. The reaction was allowed to warm to rt and stirred for another 3 h. The reaction mixture was quenched by adding acetone, and extracted with ether. The organic layer was washed with brine and dried over Na₂SO₄, then concentrated in vacuo. Purification by column chromatography on silica gel, (Hexanes/EtOAc, 4:1) gave 26 as colorless oil (7.12 g, 91% yield). [α]_{D}²³=−139.0 (c=1.0, CH₂Cl₂). ¹H NMR (CDCl₃): δ 7.45-7.26 (5H, Ar), 5.90 (1H, ddd, J_{2,3}=7.4, J_{2,1a}=16.8. J_{2,1b}=10.4 Hz, H-2), 5.47 (1H, dd, J_{1a,1b}=1.5 Hz, H-1a), 5.39 (1H, s, Ph-CH), 5.30 (1H, dd, H-1b), 4.50 (1H, dd, J_{3,4}=9.8 Hz, H-3), 4.34 (1H, dd, J_{7a,7b}=10.4, J_{7a,6}=5.3 Hz, H-7a), 4.19 (1H, dddd, J_{6,7b}=10.3, J_{6,5}=9.4, J_{6,—OH}=4.5 Hz, H-6), 4.03 (1H, dd, J_{4,5}=2.7 Hz, H-4), 3.68 (1H, dd, H-5), 3.58 (1H, dd, H-7b), 3.30, 3.26 (6H, 2×-OMe), 2.13 (1H, d, OH-6), 1.33, 1.31 (6H, 2×-Me). ¹³C NMR: δ 137.7-126.4 (6C, Ar), 134.0 (C-2), 119.4 (C-1), 101.6 (Ph-CH), 99.4, 98.8, 80.4 (C-5), 71.6 (C-7), 70.1 (C-3), 69.4 (C-4), 61.4 (C-6), 48.3, 48.1 (2×-OMe), 17.9, 17.8 (2×-Me). Anal. Calcd. for C₂₀H₂₈O₇: C, 63.14; H, 7.42. Found: C, 63.39; H, 7.37.

6-O-Benzyl-5,7-O-benzylidene-3,4-O-(2′,3′-dimethoxybutane-2′,3′-diyl)-D-gluco-hept-1-enitol (27)—A mixture of compound 26 (6.89 g, 0.018 mol) and 60% NaH (1.5 equiv) in DMF (100 mL) was stirred in an ice bath for 20 min. A solution of benzyl bromide (2.56 mL, 0.02 mol) in DMF (10 mL) was added, and the mixture was stirred at rt for 2 h. The reaction was quenched with ice water (50 mL) and the mixture was diluted with Et₂O (100 mL). The organic layer was washed with H₂O (50 mL) and brine (50 mL). The organic phase was dried over anhydrous Na₂SO₄, filtered, and concentrated. The crude product was purified by flash chromatography [hexanes/EtOAc, 5:1] to give compound 27 as colorless oil (7.31 g, 85%). [α]_{D}²³=−79.0 (c=1.0, CH₂Cl₂). ¹H NMR (CDCl₃): δ 7.48-7.26 (10H, Ar), 5.87 (1H, ddd, J_{2,3}=8.0, J_{2,1a}=17.2, J_{2,1b}=10.4 Hz, H-2), 5.43 (1H, dd, J_{1a,1b}=1.8 Hz, H-1a), 5.38 (1H, s, Ph-CH), 5.30 (1H, dd, H-1b), 4.60 (2H, dd, Ph-CH₂), 4.53 (1H, dd, J_{3,4}=9.8 Hz, H-3), 4.47 (1H, dd, J_{7a,7b}=10.7, J_{7a,6}=5.0 Hz, H-7a), 4.10 (1H, ddd, J_{6,7b}=10.4, J_{6,5}=9.3, Hz, H-6), 4.08 (1H, dd, J_{4,5}=1.9 Hz, H-4), 3.77 (1H, dd, H-5), 3.61 (1H, dd, H-7b), 3.24, 3.19 (6H, 2×-OMe), 1.34, 1.31 (6H, 2×-Me). ¹³C NMR: δ 138.1-126.3 (12C, Ar), 134.1 (C-2), 119.9 (C-1), 101.2 (Ph-CH), 99.5, 98.8, 78.9 (C-5), 71.7 (Ph-CH₂), 70.3 (C-3), 69.9 (C-7), 67.9 (C-4), 67.5 (C-6), 48.2, 48.0 (2×-OMe), 18.1, 18.0 (2×-Me). Anal. Calcd. for C₂₇H₃₄O₇: C, 68.92; H, 7.28. Found: C, 69.13; 1-1, 7.57.

6-O-Benzyl-5,7-O-benzylidene-3,4-O-(2′,3′-dimethoxybutane-2′,3′-diyl)-D-glycero-D-gulo-heptitol (28)—To a solution of 27 (3.2 g, 6.80 mmol) in acetone:water (9:1, 50 mL) at 0° C., were added NMO (820 mg, 5.10 mmol) and OsO₄ (340 mg, 0.034 mmol, 2.5 wt % solution in 2-methyl-2-propanol). The reaction mixture was stirred at rt for 4 h before it was quenched with a saturated solution of NaHSO₃. After stirring for an additional 15 min. the reaction mixture was extracted with ethyl acetate and the organic layer was washed with water and brine, dried, and concentrated. Chromatographic purification of the residue (hexane/EtOAc, 2:1) afforded 28 (3.02, 88%) as colorless oil. [α]_{D}²³=−116.0 (c=0.1, CH₂Cl₂). ¹H NMR (CDCl₃): δ 7.40-7.23 (10H, Ar), 5.44 (1H, s, Ph-CH), 4.63 (2H, dd, Ph-CH₂), 4.43 (1H, dd, J_{7a,7b}=10.5, J_{7a,6}=5.1 Hz, H-7a), 4.25 (1H, dd, J_{3,4}=10.0, J_{3,2}=5.1 Hz, H-3), 4.16 (1H, dd, J_{4,5}=2.5 Hz, H-4), 4.11 (1H, ddd, J_{6,5}=9.2, J_{6,7b}=10.4, H-6), 3.97 (1H, dd, H-5), 3.87 (1H, m, H-1a), 3.79 (2H, m, H-2, H-1b), 3.63 (1H, dd, H-7b), 3.25, 3.18 (6H, 2×-OMe), 2.86 (1H, OH-2), 2.33 (1H, OH-1), 1.32, 1.28 (6H, 2×Me). ¹³C NMR: δ 138.1-126.3 (12C, Ar), 101.4 (Ph-CH), 99.3, 98.9, 79.3 (C-5), 71.8 (Ph-CH₂), 70.6 (C-2), 70.1 (C-3), 69.9 (C-7), 67.7 (C-6), 67.7 (C-4), 63.8 (C-1), 48.3, 48.2 (2×-OMe), 17.8, 17.7 (2×Me). Anal. Calcd. for C₂₇H₃₆O₉: C, 64.27; H, 7.19. Found: C, 64.01; H, 7.44.

1,2,6-Tri-O-benzyl-5,7-O-benzylidene-3,4-O-(2′,3′-dimethoxybutane-2′,3′-diyl)-D-glycero-D-gulo-heptitol (30)—A mixture of compound 28 (2.90 g, 5.74 mmol) and 60% NaH (2.5 equiv) in DMF (100 mL) was stirred in an ice bath for 1 h. A solution of benzyl bromide (1.53 mL, 12.6 mmol) in DMF (10 mL) was added, and the mixture was stirred at rt for 3 h. The reaction was quenched with ice water and the mixture was diluted with Et₂O (100 mL). The organic layer was washed with H₂O and brine. The organic phase was dried over anhydrous Na₂SO₄, filtered, and concentrated. The crude product was purified by flash chromatography [hexanes/EtOAc, 5:1] to give compound 30 as a colorless oil (3.48, 88%). [α]_{D}²=−102.4 (c=1.2, CH₂Cl₂). ¹H NMR (CDCl₃): δ 7.43-7.21 (20H, Ar), 5.20 (1H, s, Ph-CH), 4.66-4.54 (6H, 3×Ph-CH₂), 4.40 (2H, m, H-3, H-7a), 4.29 (1H, dd, J_{4,5}=2.4, J_{4,3}=9.9 Hz, H-4), 4.08 (1H, ddd, J_{6,7a}=5.0, J_{6,7b}=10.2, J_{6,5}=9.5 Hz, H-6), 3.96 (1H, dd, H-5), 3.84 (1H, dd, J_{1a,2}=3.3, J_{1a,1b}=8.9 Hz, H-1a), 3.81 (1H, ddd, J_{2,3}=5.6, J_{2,1b}=5.9 Hz, H-2), 3.78 (1H, dd, H-1b), 3.53 (1H, dd, H-7b), 3.24, 3.16 (6H, 2×-OMe), 1.30, 1.28 (6H, 2×-Me). ¹³C NMR: δ 138.7-126.4 (24C, Ar), 101.2 (Ph-CH), 99.3, 98.9, 79.1 (C-5), 78.5 (C-2), 73.6, 72.5, 71.5 (Ph-ClH₂), 70.3 (C-1), 69.9 (C-7), 67.8 (C-6), 66.6 (C-4), 48.1, 47.9 (2×-OMe), 17.9 (2×Me). Anal. Calcd. for C₄₁H₄₈O₉: C, 71.91; H, 7.06. Found: C, 72.02; H, 7.24.

1,2,6-Tri-O-benzyl-3,4-O-(2′,3′-dimethoxybutane-2′,3′-diyl)-D-glycero-D-gulo-heptitol (31)—To a solution of 1,2,6-tri-O-benzyl-5,7-O-benzylidene-3,4-O-(2′,3′-dimethoxybutane-2′,3′-diyl)-D-glycero-D-gulo-heptitol (30) (3.12 g, 4.55 mmol) in MeOH (150 mL) was added p-toluenesulfonic acid (200 mg) and the reaction mixture was stirred for 4 h at rt. The reaction was then quenched by addition of excess Et₃N, and the solvents were removed under vacuum to give a pale yellow syrup that was purified by flash column chromatography to give 31 (2.18 g, 79%). [α]_{D}²³=−86.4 (c=1.0, CH₂Cl₂). ¹H NMR (CDCl₃): δ 7.33-7.26 (15H, Ar), 4.74-4.46 (6H, 3×Ph-CH₂), 4.18 (1H, dd, J_{3,4}=10.0 Hz, J_{3,2}=5.6 Hz, H-3), 4.09 (1H, dd, J_{4,5}=1.0 Hz, H-4), 4.02 (1H, dd, J_{5,6}=8.0 Hz, J_{5,5-OH}=7.9 Hz, H-5), 3.84 (3H, m, H₂-7, H-1a), 3.73 (3H, m, H-6, H-2, H-1b), 3.23, 3.15 (2×-OMe), 2.76 (1H, d, 5-OH), 2.29 (1H, dd, 7-OH), 1.29, 1.26 (2×Me). ¹³C NMR: δ 138.5-127.4 (Ar), 98.9, 98.6, 79.0 (C-2), 78.0 (C-6), 73.6, 72.6, 71.4 (3×Ph-CH₂), 70.9 (C-5), 69.4 (C-4), 69.2 (C-1), 67.1 (C-3), 61.7 (C-7), 48.4, 48.2 (2×-OMe), 17.8, 17.7 (2×-Me). Anal. Calcd. for C₃₄H₄₄O₉: C, 68.44; H, 7.43. Found: C, 68.39; H, 7.23.

1,2,6-Tri-O-benzyl-3,4-di-O-(2′,3′-dimethoxybutane-2′,3′-diyl)-D-glycero-D-gulo-heptitol-5,7-cyclic sulfate (22)—A mixture of 31 (2.0 g, 3.35 mmol) and Et₃N (1.5 mL, 15.0 mmol) in CH₂Cl₂ (100 mL) was stirred in an ice bath. Thionyl chloride (0.36 mL, 5.0 mmol) in CH₂Cl₂ (10 mL) was then added dropwise over 15 min, and the mixture was stirred for an additional 30 min. The mixture was poured into ice-cold water and extracted with CH₂Cl₂ (2×100 mL). The combined organic layers were washed with brine, and dried over Na₂SO₄. The solvent was removed under reduced pressure and the residue was purified by flash column chromatography (8:1, 5:1, 3:1 hexanes:EtOAc) to gave the diastereomeric mixture of cyclic sulfites. To a solution of the cyclic sulfites in a mixture of CH₃CN:CCl₄ (100 mL) were added sodium periodate (1.48 g, 6.95 mmol) and RuCl₃ (100 mg), followed by H₂O (20 mL). The mixture was then stirred for 2 h at rt. The reaction mixture was filtered through a silica bed and washed repeatedly with EtOAc. The volatile solvents were removed, and the aqueous solution was extracted with EtOAc (2×100 mL). The combined organic layers were washed with saturated NaCl, dried over Na₂SO₄, and evaporated under diminished pressure. The residue was purified by flash column chromatography to give 22 as a white amorphous solid (1.41 g, 63%). [α]_{D}²³=−57.3 (c=0.7, CH₂Cl₂). ¹H NMR (CDCl₃): δ 7.34-7.26 (15H, Ar), 5.11 (1H, m, H-5), 4.68-4.51 (6H, 3×Ph-CH₂), 4.39 (3H, m, H₂-7, H-6), 4.35 (1H, dd, J_{4,5}=1.9, J_{4,3}=9.8 Hz, H-4), 4.20 (1H, dd, J_{3,2}=3.6 Hz, H-3), 3.80 (1H, dd, J_{1a,1b}=9.7, J_{1a,2}=5.8 Hz, H-1a), 3.75 (1H, ddd, H-2), 3.67 (1H, dd, J_{1b,2}=5.0 Hz, H-1b), 3.22, 3.12 (6H, 2×-OMe), 1.32, 1.26 (6H, 2×-Me). ¹³C NMR: δ 138.4-127.3 (18C, Ar), 99.6, 98.9, 84.0 (C-5), 77.5 (C-2), 73.6, 72.7, 72.5 (Ph-CH₂), 72.0 (C-6), 69.2 (C-1), 67.1 (C-7), 68.8 (C-3), 65.9 (C-4), 48.4, 48.2 (2×-OMe), 17.8, 17.6 (2×-Me). Anal. Calcd. for C₃₄H₄₂O₁₁S: C, 61.99; H, 6.43. Found: C, 61.76; H, 6.44.

2,6,7-Tri-O-benzyl-4,5-di-O-(2′,3′-dimethoxybutane-2′,3′-diyl)-D-glycero-L-gulo-heptitol-1,3-cyclic sulfate (23)—A mixture of 30 (2.0 g, 3.35 mmol) and Et₃N (1.35 mL, 13.4 mmol) in CH₂Cl₂ (80 mL) was stirred at 0° C. Thionyl chloride (0.37 mL, 5.0 mmol) in CH₂Cl₂ (5 mL) was then added dropwise over 20 min, and the mixture was stirred for an additional 30 min. The mixture was poured into ice-cold water and extracted with CH₂Cl₂ (2×100 mL). The combined organic layers were washed with brine, dried over Na₂SO₄, and concentrated. Column chromatography (hexanes:EtOAc, 8:1, 5:1, 3:1) gave the diastereomeric mixture of cyclic sulfites. To a solution of the cyclic sulfites in a mixture of CH₃CN:CCl₄ (1:1, 60 mL) were added sodium periodate (0.70 g, 3.35 mmol) and RuCl₃ (80 mg), followed by H₂O (10 mL). The mixture was then stirred for 2 h at rt. The reaction mixture was filtered through a silica bed and washed repeatedly with EtOAc. The volatile solvents were removed, and the aqueous solution was extracted with EtOAc (2×100 mL). The combined organic layer was washed with saturated NaCl, dried over Na₂SO₄, and evaporated under diminished pressure. The residue was purified by flash column chromatography to give 23 as a white solid (1.41 g, 63%). mp 124-126° C.; [α_{D}²³]=−128.0 (c=1.0, CH₂Cl₂). ¹H NMR (CDCl₃): δ 7.36-7.25 (15H, Ar), 4.80-4.52 (6H, 3×Ph-CH₂), 4.47 (1H, dd, J_{4,3}=1.8, J_{4,5}=10.2 Hz, H-4), 4.45 (1H, dd, J_{3,2}=9.8, Hz, H-3), 4.41 (1H, dd, J_{1a,1b}=10.4, J_{1a,2}=4.6 Hz, H-1a), 4.32 (1H, ddd, J_{2,1b}=9.7 Hz, H-2), 4.24 (1H, dd, H-1b), 4.07 (1H, dd, J_{5,6}=2.6 Hz, H-5), 3.87 (1H, dd, J_{7a,7b}=10.0, J_{7a,6}=6.0 Hz, H-7a), 3.80 (1H, dd, J_{7b,6}=4.7 Hz, H-7b), 3.76 (1H, m, H-6), 3.15, 3.13 (2×-OMe), 1.30, 1.28 (2×-Me). ¹³C NMR: δ 138.3-127.4 (18C, Ar), 99.7, 99.3, 83.2 (C-3), 73.9 (C-6), 73.6, 72.7, 72.5 (Ph-CH₂), 71.8 (C-1), 69.6 (C-7), 66.9 (C-2), 66.8 (C-5), 64.8 (C-4), 48.4, 48.2 (2×-OMe), 17.8, 17.7 (2×Me). Anal. Calcd. for C₃₄H₄₂O₁₁S: C, 61.99; H, 6.43. Found: C, 61.76; H, 6.44.

### 9.2.2 Preparation of Thioarabinatol 21

6-O-benzyl-5,7-O-benzylidene-1-O-(tert-butyldimethylsilyl)-3,4-O-(2′,3′-dimethoxybutane-2′,3′-diyl)-D-glycero-D-gulo-heptitol (32)—A mixture of 28 (3.6 g, 7.13 mmol), imidazole (1.42 g, 21.0 mmol), and TBDMSCl (1.18 g, 7.85 mmol) in dry DMF (80 mL) was stirred at 0° C. under N₂ for 2 h. The reaction was quenched by the addition of ice cold water, and the reaction mixture was partitioned between Et₂O (200 mL) and H₂O (100 mL). The separated organic phase was washed with H₂O (50 mL) and brine (50 mL). The organic layer was dried over anhydrous Na₂SO₄ and concentrated. The residue was purified by flash chromatography (hexanes/EtOAc, 3:1) to give 32 as a colorless oil (3.98 g, 90%). [α]_{D}²³=−73.0 (c=1.5, CH₂Cl₂). ¹H NMR (CDCl₃): δ 7.41-7.21 (10H, Ar), 5.39 (1H, s, Ph-CH), 4.55 (2H, dd, Ph-CH₂), 4.40 (1H, dd, J_{7a,7b}=10.6, J_{7a,6}=5.0 Hz, H-7a), 4.25 (1H, dd, J_{5,4}=2.3, J_{5,6}=9.4 Hz, H-6), 4.15 (1H, dd, J_{4,3}=9.7 Hz, H-4), 4.09 (2H, m, H-3, H-6), 3.83 (1H, dd, J_{1a,1b}=9.5, J_{1a,2}=4.4 Hz, H-1a), 3.70 (1H, dd, J_{1b,2}=3.8 Hz, H-1b), 3.65 (1H, ddd, J_{2,—OH}=7.5 Hz, H-2), 3.59 (1H, dd, J_{7b,6}=10.3 Hz, H-7b), 3.17, 3.11 (6H, 2×-OMe), 2.50 (1H, d, OH-2), 1.26, 1.21 (6H, 2×-Me), 0.80 (9H, s, TBDMS), 0.00 (6H, s, TBDMS). ¹³C NMR: δ 143.6-131.5 (12C, Ar), 101.3 (Ph-CH), 99.3, 98.8 (BDA), 79.6 (C-5), 71.9 (Ph-CH₂), 71.7 (C-2), 70.2 (C-7), 68.6 (C-4), 68.1 (C-6), 67.5 (C-3), 63.3 (C-1), 48.5, 48.3 (2×-OMe), 26.2 (TBDMS), 16.6 (TBDMS), 18.1, 18.0 (2×-Me), −5.0, −5.5 (TBDMS). Anal. Calcd. for C₃₃H₅₀O₉Si: C, 64.05; H, 8.14. Found: C, 64.17; H, 8.38.

2-O-benzyl-1,3-O-benzylidene-7-O-(tert-butyldimethylsilyl)-4,5-O-(2′,3′-dimethoxy butane-2′,3′-diyl)-6-O-(4-nitrobenzoyl)-D-glycero-L-gulo-heptitol (33)—A solution of 32 (3.72 g, 6.01 mmol) in THF (60 mL) containing p-nitrobenzoic acid (3.0 g, 18.0 mmol) and triphenylphosphine (4.7 g, 18.0 mmol) was cooled to 0° C. A solution of diisopropyl azodicarboxylate (3.64 mL, 18.0 mmol) in THF (30 mL) was added to the mixture over 2 h. After stirring for 20 h at ambient temperature, the reaction mixture was concentrated and then partitioned between Et₂O (200 mL) and H₂O (100 mL). The organic phase was washed with saturated aqueous NaHCO₃ (3×50 mL), followed by brine (50 mL). The organic layer was dried over anhydrous Na₂SO₄ and concentrated under vacuum. The residue was purified by flash chromatography (hexanes/EtOAc, 3:1) to give 33 as colorless oil (2.96 g, 64%). [α]_{D}²³=−53.1 (c=1.5, CH₂Cl₂). ¹H NMR (CDCl₃): δ 8.18-6.99 (14H, Ar), 5.41 (1H, s, Ph-CH), 5.33 (1H, ddd, J_{6,5}=1.9, J_{6,7a}=6.8, J_{6,7b}=6.6 Hz, H-6), 4.52 (1H, d, Ph-CH₂), 4.49 (1H, dd. J_{5,4}=10.0 Hz, H-5), 4.44 (1H, J_{1a,1b}=10.4, J_{1a,2}=5.0 Hz, H-1a), 4.42 (1H, d, Ph-CH₂), 4.26 (1H, dd, J_{4,3}=2.0 Hz, H-4), 4.05 (1H, ddd, J_{2,3}=9.2, J_{2,1b}=10.4 Hz, H-2), 4.00 (1H, dd, J_{7a,7b}=10.0, J_{7a,6}=6.8 Hz, H-7a), 3.91 (1H, dd, J_{7b,6}=6.6 Hz, H-7b), 3.82 (1H, dd, H-3), 3.62 (1H, dd, H-1b), 3.27, 3.09 (6H, 2×-OMe), 1.33, 1.32 (6H, 2×-Me), 0.79 (9H, s, TBDMS), 0.03, 0.00 (6H, s, TBDMS). ¹³C NMR: δ 164.6 (C═O), 150.4-123.4 (18C, Ar), 101.1 (Ph-CH), 99.1, 98.8, 78.1 (C-3), 73.7 (C-6), 70.9 (Ph-CH₂), 69.6 (C-1), 66.8 (C-2), 65.2 (C-5), 64.9 (C-4), 59.9 (C-7), 47.9 (2×-OMe), 25.6 (TBDMS), 18.0 (TBDMS), 17.6 (2×-Me), —5.4, −5.5 (TBDMS). Anal. Calcd. for C₃₉H₅₃NO₁₁Si: C, 63.31; H, 7.22. Found: C, 63.26; H, 7.12.

2-O-benzyl-1,3-O-benzylidine-7-O-(tert-butyldimethylsilyl)-4,5-O-(2′,3′-dimethoxy butane-2′,3′-diyl)-D-glycero-L-gulo-heptitol (34)—Compound 33 (2.70 g, 3.51 mmol) was dissolved in MeOH (50 mL) and 1 N NaOMe/MeOH (1.0 mL) was added. The mixture was stirred at rt for 1 h and then Rexyn 101 (H⁺) was added to adjust the pH to 7. The solvent was removed and the residue was partitioned between Et₂O (150 mL) and H₂O (100 mL). The organic layer was washed with brine (50 mL), dried over anhydrous Na₂SO₄, and concentrated. The residue was purified to give 34 as a white foam (2.05 g, 94%). [α]_{D}²³=−66.4 (c=1.6, CH₂Cl₂). ¹H NMR (CDCl₃): δ 7.46-31 (Ar), 5.43 (1H, s, Ph-CH), 4.61 (2H, s, Ph-CH₂), 4.46 (1H, dd, J_{4,3}=2.4, J_{4,5}=10.0 Hz, H-4), 4.42 (1H, dd, J_{1b,1a}=10.5, 5.0 Hz, H-1b), 4.22 (1H, dd, J_{5,6}=1.3 Hz, H-5), 4.11 (1H, ddd, J_{2,1b}=10.4, J_{2,3}=9.2 Hz, H-2), 3.96 (1H, dd, H-3), 3.77 (1H, m, H-6), 3.71 (2H, m, H₂-7), 3.66 (1H, dd, H-1a), 3.20, 3.15 (2×-OMe), 2.35 (1H, d, J_{—OH,6}=7.0 Hz, OH-6), 1.31, 1.27 (2×Me), 0.80 (9H, TBDMS), 0.016, 0.00 (TBDMS). ¹³C NMR: δ 138.9-126.9 (12C, Ar), 101.1 (Ph-CH), 99.9, 99.5, 79.2 (C-2), 72.2 (Ph-CH₂), 70.8 (C-1), 70.5 (C-6), 68.2 (C-2), 67.1 (C-5), 66.0 (C-4), 64.1 (C-7), 48.7, 48.5 (2×-OMe), 26.5 (TBDMS), 18.9 (TBDMS), 18.5, 18.4 (2×Me), −4.57, −4.65 (TBDMS). Anal. Calcd. for C₃₃H₅₀O₉Si: C, 64.05; H, 8.14. Found: C, 64.02; H, 8.31.

2-O-Benzyl-1,3-O-benzylidene-4,5-O-(2′,3′-dimethoxybutane-2′,3′-diyl)-D-glycero-L-gulo-heptitol (29)—TBAF (1.0 M solution in THF, 3.90 mL, 3.9 mmol) was added dropwise to a stirred solution of the TBDMS-protected alcohol 34 (1.96 g, 3.25 mmol) in THF (30 mL) at rt. After 2 h at rt, the reaction mixture was concentrated and the residue was purified by flash chromatography (EtOAc:hexane=3:7) to yield 29 as a white crystalline solid (1.48 g, 92%). mp 118-120° C.; [α]_{D}²³−128.4 (c=1.3, CH₂Cl₂). ¹H NMR (CDCl₃): δ 7.43-7.26 (10H, Ar), 5.43 (1H, s, Ph-CH), 4.62 (2H, dd, Ph-CH₂), 4.46 (1H, dd, J_{4,3}=2.7, J_{4,5}=9.7 Hz, H-4), 4.44 (1H, dd, J_{1a,1b}=10.4, J_{1a,2}=5.0 Hz, H-1a), 4.17 (1H, dd, J_{5,6}=1.8 Hz, H-5), 4.12 (1H, ddd, J_{2,3}=9.2, J_{2,1b}=10.4, H-2), 3.99 (1H, dd, H-3), 3.85 (1H, ddd, J_{7a,7b}=11.0, J_{7a,6}=5.6, J_{7a,—OH}=2.0, H-7a), 3.80 (1H, m, H-6), 3.68 (1H, ddd, J_{7b,6}=10.0, J_{7b,—OH}=9.8 Hz, H-7b), 3.64 (1H, dd, H-1b), 3.21, 3.16 (6H, 2×-OMe), 2.63 (1H, d, OH-6), 2.37 (1H, dd, OH-7), 1.31, 1.29 (6H, 2×Me). ¹³C NMR: δ 138.2-126.3 (12C, Ar), 101.3 (Ph-CH), 99.4, 99.3, 78.7 (C-3), 71.7 (Ph-CH₂), 70.2 (C-1), 69.9 (C-5), 69.3 (C-6), 67.6 (C-2), 65.5 (C-7), 65.3 (C-4), 48.2, 48.1 (2×-OMe), 17.9 (2×-Me). Anal. Calcd. for C₂₇H₃₆O₉: C, 64.27; H, 7.19. Found: C, 64.63; H, 7.44.

2,6,7-Tri-O-benzyl-1,3-O-benzylidene-4,3′-dimethoxybutane-2′,3′-diyl)-D-glycero-L-gulo-heptitol (35)—A mixture of compound 29 (1.40 g, 2.77 mmol) and 60% NaH (1.5 equiv) in DMF (100 mL) was stirred at 0° C. for 1 h. A solution of benzyl bromide (0.74 mL, 6.01 mmol) in DMF (5 mL) was added, and the mixture was stirred at rt for 2 h. The reaction was quenched by addition of ice cold water (50 mL) and the mixture was diluted with Et₂O (150 mL). The organic layer was washed with H₂O (50 mL) and brine (50 mL). The organic phase was dried over anhydrous Na₂SO₄, filtered, and concentrated. The crude product was purified by flash chromatography [hexanes/EtOAc, 5:1] to give compound 35 as a white crystalline solid (1.76 g, 92%). mp 104-106° C.; [α]_{D}²³=−90.6 (c=0.7, CH₂Cl₂). ¹H NMR (CDCl₃): δ 7.38-7.23 (20H, Ar), 4.88 (1H, s, Ph-CH), 4.84-4.55 (6H, 3×Ph-CH₂), 4.42 (1H, dd, J_{4,3}=2.7, J_{4,5}=9.7 Hz, H-4), 4.37 (1H, dd, J_{1a,1b}=10.5, J_{1a,2}=5.0 Hz, H-1a), 4.23 (1H, dd, J_{5,6}=2.2 Hz, H-5), 3.98 (1H, dd, J_{2,1b}=10.4 Hz, H-2), 3.90 (2H, d, J_{7,6}=5.6 Hz, H₂-7), 3.79 (1H, dt, H-6), 3.33 (1H, dd, H-1b), 3.13 (1H, m, H-3), 3.13 (6H, 2×-OMe), 1.30, 1.28 (6H, 2×Me). ¹³C NMR: δ 138.6-126.4 (24C, Ar), 100.9 (Ph-CH), 99.3, 99.3, 78.1 (C-3), 73.3, 71.3, 71.2 (Ph-CH₂), 73.3 (C-6), 69.6 (C-1), 69.5 (C-7), 67.7 (C-5), 67.4 (C-2), 65.4 (C-4), 48.0, 47.9 (2×-OMe), 18.0, 17.9 (2×Me). Anal. Calcd. for C₄₁H₄₈O₉: C, 71.91; 1-1, 7.06. Found: C, 71.99; H, 7.19.

2,6,7-Tri-O-benzyl-4,5-di-O-(2′,3′-dimethoxybutane-2′,3′-diyl)-D-glycero-L-gulo-heptitol (36)—To a solution of 35 (1.60 g, 9.6 mmol) in MeOH (100 mL), p-toluenesulfonic acid (200 mg) was added, and the reaction mixture was stirred for 6 h at rt. The reaction was then quenched by addition of excess Et₃N, the solvents were removed, and the yellow syrup was purified by flash column chromatography to give 21 as a white amorphous solid (1.08 g, 77%). [α]_{D}²³=−91.6 (c=0.6, CH₂Cl₂). ¹H NMR (CDCl₃): δ 7.33-7.21 (15H, Ar), 4.70-4.48 (6H, 3×Ph-CH₂), 4.35 (1H, d, J_{4,5}=10.2 Hz, H-4), 4.14 (1H, dd, J_{5,6}=2.0 Hz, H-5), 3.90 (2H, m, H₂-1), 3.76 (3H, m, H-2, H-6, H-7a), 3.65 (2H, m, H-3, H-7b), 3.19, 3.16 (2×-OMe), 2.65 (1H, J=8.9 3-OH), 2.25 (1H, dd, J=5.1, 7.6 Hz, 1-OH), 1.30, 1.29 (2×Me). ¹³C NMR: δ 137.9-126.8 (Ar), 99.1, 99.0, 78.3 (C-6), 75.3 (C-6), 73.4, 72.5, 71.7 (3×Ph-CH₂), 69.9 (C-2), 69.8 (C-7), 67.8 (C-5), 66.9 (C-4), 61.4 (C-1), 48.4, 48.1 (2×-OMe), 17.8, 17.7 (2×-Me). Anal. Calcd. for C₃₄H₄₄O₉: C, 68.44; H, 7.43. Found: C, 68.59; H, 7.39.

### 9.2.3 Preparation of Compounds 17 and 18

2,3,5-Tri-O-p-methoxybenzyl-1,4-dideoxy-1,4-[[2S,3S,4R,5S,6S]-2,6,7-tri-O-benzyl-4,5-di-O-(2′,3′-dimethoxybutane-2′,3′-diyl)-3-(sulfooxy)-heptyl]-(R)-epi-sulfoniumylidine]-D-arabinitol Inner salt (37)—The thioarabinitol 21 (210 mg, 0.42 mmol) and the cyclic sulfate 22 (308 mg, 0.46 mmol) were added to 1,1,1,3,3,3-hexafluoroisopropanol (HFIP) (3 mL) containing anhydrous K₂CO₃ (40 mg). The mixture was stirred in a sealed tube at 72° C. for 48 h. The solvent was removed under reduced pressure, and the residue was purified by flash column chromatography (3:1 hexanes/EtOAc and then 20:1, 15:1 EtOAc/MeOH). The coupled product, 37 was obtained as a white amorphous solid (258 mg, 52%). [α]_{D}²³=−82.0 (c=0.5, CH₂Cl₂). ¹H NMR (acetone-d₆): δ 7.44-6.84 (27H, Ar), 4.93 (1H, J_{3′,4′}=1.7, J_{3′,2′}=5.1 Hz, H-3′), 4.85-4.22 (12H, 3×Ph-CH₂, 3×Ph-CH₂), 4.68 (1H, m, H-2), 4.57 (1H, m, H-5′), 4.45 (1H, m, H-3), 4.37 (1H, dd, J_{1a′,1b′}=13.5, J_{1a′,2′}=3.9 Hz, H-1a′), 4.35 (1H, m, H-6′), 4.30 (1H, dd, J_{4′,5′}=10.0 Hz, H-4′), 4.23 (1H, m, H-2′), 4.20 (1H, dd, J_{1a,1b}=13.5, J_{1a,2}=2.6 Hz, H-1a), 4.15 (1H, dd, =4.4 Hz, H-1b′), 4.06 (1H, dd, H-4), 4.00 (1H, dd, J_{1b,2}=3.9 Hz, H-1b), 3.94 (1H, dd, J_{7a′,7b′}=9.9 Hz, J_{7a′,6′}=6.5 Hz, H-7a′), 3.80, 3.79 (3×-OMe), 3.70 (1H, dd, J_{5a,5b}=10.0, J_{5a,4}=6.9 Hz, H-5a), 3.61 (1H, dd, J_{7b,6}=5.4 Hz, H-7b′), 3.54 (1H, dd, J_{5b,4}=8.5, H-5b), 3.20, 3.09 (2×-OMe), 1.19, 1.18 (2×-Me). ¹³C NMR: δ 159.9-113.8 (32C, Ar), 99.2, 98.4, 83.3 (C-3), 82.2 (C-2), 76.2 (C-6′), 75.6 (C-2′), 73.4 (C-3′), 72.9, 72.6, 72.0, 71.7, 71.3, 71.3 (3×Ph-CH₂, 3×Ph-CH₂), 69.8 (C-7′), 68.9 (C-4′), 68.8 (C-5′), 66.8 (C-5), 65.7 (C-4), 54.9, 54.8 (3×-OMe), 49.4 (C-1′), 49.2 (C-1), 47.9, 47.1 (2×-OMe), 17.4, 17.3 (2×Me). Anal. Calcd. for C₆₃H₇₆O₁₇S₂: C, 64.71; H, 6.55. Found: C, 64.38; H, 6.52.

2,3,5-Tri-O-p-methoxybenzyl-1,4-dideoxy-1,4-[[2S,3S,4R,5S,6R]-2,6,7-tri-O-benzyl-4,5-di-O-(2′,3′-dimethoxybutane-2′,3′-diyl)-3-(sulfooxy)-heptyl]-(R)-epi-sulfoniumylidine]-D-arabinitol Inner salt (38)—To HFIP (3 mL) were added the thioarabinitol 21 (238 mg, 0.46 mmol), the cyclic sulfate 23 (324 mg, 0.49 mmol), and anhydrous K₂CO₃ (40 mg). The mixture was stirred in a sealed tube at 72° C. for 72 h. The solvent was removed under reduced pressure, and the residue was purified by flash column chromatography (3:1 hexanes:EtOAc and then 15:1 EtOAc:MeOH) to give 38 as a white amorphous solids (265 mg, 49%). [α]_{D}²³=−54.0 (c=0.5, CH₂Cl₂). ¹H NMR (acetone-d₆): δ 7.42-6.84 (27H, Ar), 4.96-4.12 (12H, 3×Ph-CH₂, 3×Ph-CH₂), 4.90 (1H, J_{3′,4′}=1.7 Hz, J_{3′,2′}=6.4 Hz, H-3′), 4.74 (1H, m, H-6′), 4.69 (1H, m, H-2), 4.52 (1H, dd, J_{4′,5′}=9.6 Hz, H-4′), 4.46 (1H, m, H-3), 4.39 (3H, m, H₂-1′, H-2′), 4.35 (1H, m, H-5′), 4.18 (1H, m, H-1a), 4.01 (2H, m, H-4, H-1b), 3.95 (1H, dd, J_{7a′,6′}=7.7, J_{7a′,7b′}10.6 Hz, H-7a′), 3.81 (1H, dd, =3.8 Hz, H-7b′), 3.80, 3.78, 3.77 (3×-OMe), 3.63 (1H, dd, J_{5a,5b}=10.0, J_{5a,4}=4.7 Hz, H-5a), 3.50 (1H, dd, J_{5b,4}=8.0 Hz, H-5b), 3.14, 3.06 (2×-OMe), 1.82 (2×Me). ¹³C NMR: δ 159.9-113.8 (32C, Ar), 99.1, 98.6, 83.4 (C-3), 82.1 (C-2), 75.9 (C-6′), 75.1 (C-2′), 73.3 (C-3′), 73.1, 72.6, 72.4, 71.7, 71.7, 71.4 (3×Ph-CH₂, 3×Ph-CH₂), 71.3 (C-7′), 69.1 (C-5′), 67.9 (C-4′), 66.7 (C-5), 65.3 (C-4), 54.8, 54.8 (3×-OMe), 49.2 (C-1′), 48.9 (C-1), 47.9, 47.3 (2×-OMe), 17.5, 17.4 (2×-Me). Anal. Calcd. for C₆₃H₇₆O₁₇S₂: C, 64.71; H, 6.55. Found: C, 64.93; H, 6.65.

1,4-Dideoxy-1,4-[[2S,3S,4R,5S,6S]-2,4,5,6-pentahydroxy-3-(sulfooxy)-heptyl]-(R)-epi-sulfoniumylidine]-D-arabinitol Inner salt (17)—Compound 37 (78 mg, 0.075 mmol) was dissolved in a mixture of CH₃COOH:H₂O (20 mL, 4:1) and the solution was stirred with 10% Pd/C (100 mg) under 100 psi of H₂ for 48 h. The catalyst was removed by filtration through a bed of silica, and washed with water (25 mL). The solvents were removed under reduced pressure and 80% aqueous TFA (10 ml) was added. The mixture was stirred at rt for 2 h. The solvents were then evaporated under diminished pressure and the residue was purified by flash column chromatography to give 17 as a white crystalline solid. mp 164-166. [α]_{D}²³=+18.3 (c=0.6, MeOH). ¹H NMR (D₂O): δ 4.60 (1H, dd, J_{2,1}=3.4, J_{2,3}=3.2 Hz, H-2), 4.36 (1H, dd, J_{3′,2′}=7.1, J_{3′,4′}=2.7 Hz, H-3′), 4.32 (1H, ddd, J_{2′,1′a}=3.2, J_{2′b′}=7.6 Hz, H-2′), 4.30 (1H, dd, J_{3,4}=3.1 Hz, H-3), 4.02 (1H, t, J_{4′,5′}=2.7 Hz, H-4′), 3.95 (1H, dd, J_{5a,5b}=11.1, J_{5a,4}=4.9 Hz, H-5a), 3.93 (1H, ddd, H-4), 3.88 (1H, dd, J_{1a′,1b′}=13.5 Hz, H-1a′), 3.81 (1H, dd, J_{5b,4}=7.6 Hz, H-5b), 3.72 (1H, dd, H-1b′), 3.71 (2H, d, (H₂-1), 3.68 (1H, dd, J_{5′,6′}=7.4 Hz, H-5′), 3.65 (1H, J_{7a′,6′}=3.2, J_{7a′,7b′}=11.2 Hz, H-7a′), 3.61 (1H, ddd, H-6′), 3.50 (1H, dd, J_{7b′,6′}=5.6 Hz, H-7b′). ¹³C NMR: δ 81.1 (C-3′), 77.8 (C-3), 76.8 (C-2), 71.4 (C-5′), 71.0 (C-6′), 70.0 (C-4), 67.7 (C-4′), 66.7 (C-2′), 62.6 (C-7′), 59.1 (C-5), 50.2 (C-1′), 47.8 (C-1). HRMS Calcd for C₁₂H₂₄O₁₂NaS₂ (M+Na): 447.0601. Found: 447.0601.

1,4-Dideoxy-1,4-[[2S,3S,4R,5S,6R]-2,4,5,6-pentahydroxy-3-(sulfooxy)-heptyl]-(R)-epi-sulfoniumylidine]-D-arabinitol Inner salt (18)—The sulfonium salt 38 (240 mg, 0.212 mmol) was deprotected following the same procedure that was used for compound 37, to give compound 18 as a crystalline solid. mp 169-171; [α]_{D}²³=+12.0 (c=0.5, MeOH). ¹H NMR (D₂O): δ 4.610 (1H, dd, J_{2,1}=3.4, J_{2,3}=3.2 Hz, H-2), 4.35 (2H, m, H-2′, H-3′), 4.32 (1H, dd, J_{3,4}=3.0 Hz, H-3), 3.98 (1H, dd, J_{5a,5b}=10.4, J_{5a,4}=4.9 Hz, H-5a), 3.95 (3H, m, H-4, H-4′, H-1a′), 3.85-3.76 (3H, m, H-5b, H-6′, H-1b′), 3.74 (2H, d, H₂-1), 3.69 (1H, dd, J_{5′,6′}=7.8 J_{5′,4′}=2.2 Hz, H-5′), 3.52 (2H, d, J_{7′,6′}=5.4, H-7′). ¹³C NMR: δ 78.9 (C-3′), 77.8 (C-3), 76.8 (C-2), 70.8 (C-5′), 71.7 (C-6′), 70.1 (C-4), 69.2 (C-4′), 66.6 (C-2′), 63.6 (C-7′), 59.2 (C-5), 50.4 (C-1′), 47.9 (C-1). HRMS Calcd for C₁₂H₂₄O₁₂NaS₂ (M+Na): 447.0601. Found: 447.0589.

### 9.3 Synthesis of Compounds 19 and 20

1,3-O-Benzylidene-2,5-O-methylene-D-mannitol (43)³⁴ —Compound 43 was prepared from 1,3:4,6-di-O-benzylidene-D-mannitol (42) by using the literature methods with some variations. Thus, compound 42³² was converted into 1,3:4,6-di-O-benzylidene-2,5-O-methylene-D-mannitol as described.³³ The product was then treated with PTSA to yield compound 43 as described below. To a solution of 1,3:4,6-di-O-benzylidene-2,5-O-methylene-D-mannitol (5.00 g, 13.51 mmol) in MeOH (250 mL) was added PTSA (200 mg), and the reaction mixture was stirred at 70° C. for 2 h. The reaction mixture was then quenched by addition of Et₃N (2 mL), and the solvents were removed under vacuum to give a colorless solid. The solids were dissolved in ethyl acetate (75 mL) and filtered, and the filtrate was concentrated to give the crude 1,3-O-Benzylidene-2,5-O-methylene-D-mannitol. The undissolved solids (˜1.1 g, 5.67 mmol, of 2,5-O-methylene-D-mannitol) were mixed with dry DMF (20 mL), benzaldehyde dimethylacetal (0.849 mL, 5.67 mmol), and PTSA (50 mg). The resulting reaction mixture was heated at 60° C. under a rotary evaporator vacuum for 2 h. The reaction was neutralized by the addition of Et₃N (1 mL), and the solvents were evaporated to give a crude product. The combined crude products were diluted with ethyl acetate (200 mL) and washed with water (150 mL) and brine (150 mL). The organic solution was dried (Na₂SO₄) and concentrated, and the crude product was purified by flash column chromatography (hexanes/EtOAc 3:7) to give 43³⁴ in 65% (2.47 g) over the two steps.

4-O-Benzyl-1,3-O-benzylidene-2,5-O-methylene-D-mannitol (44)—To a mixture of 43 (2.50 g, 8.86 mmol), and imidazole (1.45 g, 21.3 mmol), in dry DMF (30 mL) was added portionwise TBDMSCl (1.46 g, 9.70 mmol) and the mixture was stirred at 0° C. under nitrogen for 2 h. The reaction was quenched by the addition of ice-cold water (25 mL), and the reaction mixture was partitioned between Et₂O (200 mL) and water (100 mL). The separated organic solution was dried (Na₂SO₄) and concentrated on a rotary evaporator to give a crude product which was directly treated in the next step without further purification. The crude product was kept under high vacuum for 1 h, then dissolved in dry DMF (50 mL), the reaction mixture was cooled with an ice bath, and 60% NaH (1.06 g, 26.5 mmol) was added. A solution of benzyl bromide (3.16 mL, 26.5 mmol) was added, and the solution was stirred at rt for 1 h. The mixture was added to ice-water (150 mL) and extracted with Et₂O (3×100 mL). The organic solution was dried (Na₂SO₄) and concentrated to give a crude product. The crude residue was dissolved in THF (50 mL) and then TBAF (1.0 M solution in THF, 8.9 mL, 9.0 mmol) was added. After 20 h at rt, the reaction mixture was concentrated and the residue was purified by flash chromatography (hexanes/EtOAc 2:3) to yield 44 as a colorless solid (2.04 g, 62%). Mp 150-152° C.; [α]_{D}²³=−26.5° (c=1.0, CH₂Cl₂). ¹H NMR (CDCl₃): δ 7.54-7.28 (10H, m, Ar), 5.60 (1H, s, Ph-CH), 4.95 and 4.71 (2H, 2d, J_{AB}=11.0 Hz, Ph-CH₂), 4.90 and 4.83 (2H, 2 d, J_{AB}=4.2 Hz, O—CH₂—O), 4.35 (1H, dd, J_{1a,1b}=10.8, J_{1a,2}=5.4 Hz, H-1a), 3.94 (1H, m, H-6a), 3.86 (1H, dd, J_{2,3}=9.3, J_{3,4}=7.2 Hz, H-3), 3.81 (1H, td, H-2), 3.77-3.73 (3H, m, H-1b, H-5, H-6b), 3.69 (1H, dd, J_{4,5}=9.6 Hz, H-4), 2.01 (1H, t, J_{1ab,OH}=6.6 Hz, —OH). ¹³C NMR (CDCl₃): δ 138.0-126.0 (m, Ar), 100.7 (Ph-CH), 93.2 (O—CH₂—O), 86.3 (C-3), 79.7 (C-4), 75.1 (Ph-CH₂), 75.0 (C-5), 69.3 (C-1), 64.2 (C-2), 63.1 (C-6). HRMS Calcd for C₂₁H₂₅O₆ (M+H): 373.1651. Found: 373.1653.

4-O-Benzyl-1,3-O-benzylidene-2,5-O-methylene-D-manno-hep-6-enitol (45)—Compound 44 (2.00 g, 5.37 mmol) was dissolved in dry CH₂Cl₂ (30 mL), Dess Martin periodinane (2.48 g, 5.90 mmol) and NaHCO₃ (2.03 g, 24.16 mmol) were added, and the reaction mixture was stirred at rt for 15 min, then diluted with ether (100 mL) and poured into saturated aqueous NaHCO₃ (100 mL) containing a sevenfold excess of Na₂S₂O₃. The mixture was stirred to dissolve the solid, and the layers were separated. The ether layer was dried (Na₂SO₄) and the solvents were removed under vacuum to give the aldehyde that was further dried under high vacuum for 1 h. n-BuLi (n-hexane solution, 8.0 mmol, 1.5 equiv) was added dropwise to a solution of methyltriphenylphosphonium bromide (2.3 g, 6.44 mmol) in dry THF (20 mL) at −78° C. under nitrogen. The mixture was stirred for 1 h at the same temperature. A solution of the previously made aldehyde in dry THF (10 mL) was introduced into the solution at −78° C., and the resulting solution was allowed to warm to rt and stirred overnight. The reaction mixture was quenched by adding acetone (1 mL), and extracted with ether (3×100 mL). The organic layer was washed with brine, dried (Na₂SO₄), and concentrated in vacuo. Purification by column chromatography on silica gel (hexanes/EtOAc 4:1) gave 45 (1.1 g, 56%) as a colorless solid. Mp 133-135° C.; [α]_{D}²³=−48.5° (c=1.0, CH₂Cl₂). ¹H NMR (CDCl₃): δ 7.54-7.28 (10H, m, Ar), 6.10 (1H, ddd, J_{5,6}=6.0, J_{6,7b}=10.8, J_{6,7a}=17.0 Hz, H-6), 5.60 (1H, s, Ph-CH), 5.46 (1H, dd J_{7a,7b}=1.2 Hz, H-7a), 5.31 (1H, dd, H-7b), 4.92 and 4.84 (2H, 2d, J_{AB}=4.2 Hz, O—CH₂—O), 4.88 and 4.67 (2H, 2 d. J_{AB}=10.8 Hz, Ph-CH₂), 4.36 (1H, dd, J_{1a,1b}=10.2, J_{1a,2}=4.2 Hz, H-1a), 4.17 (1H, dd, J_{4,5}=9.6 Hz, H-5), 3.88-3.82 (2H, m, H-2, H-3), 3.75 (1H, t, J_{1b,2}=9.6 Hz, H-1b), 3.50 (1H, dd, J_{3,4}=7.8 Hz, H-4). ¹³C NMR (CDCl₃): δ 138.2-126.0 (m, Ar), 135.5 (C-6), 116.9 (C-7), 100.7 (Ph-CH), 92.9 (O—CH₂—O), 86.1 (C-3), 83.2 (C-4), 75.6 (C-5), 75.3 (Ph-CH₂), 69.3 (C-1), 64.1 (C-2). HRMS Calcd for C₂₂H₂₅O₅ (M+H): 369.1702. Found: 369.1697.

4-O-Benzyl-1,3-O-benzylidene-2,5-O-methylene-D-glycero-D-man no-heptitol (46)—To a solution of 45 (1.0 g, 2.71 mmol) in acetone:water (9:1, 20 mL) at rt were added NMO (N-methylmorpholine-N-oxide) (348 mg, 2.97 mmol) and OsO₄ (3.4 mg, 0.01 mmol, 2.5 wt % solution in 2-methyl-2-propanol). The reaction mixture was stirred at rt for 30 h before it was quenched with a saturated solution of NaHSO₃ (5 mL). After being stirred for an additional 15 min the reaction mixture was concentrated under reduced pressure, then extracted with ethyl acetate (3×100 mL), and the organic layer was washed with water (50 mL) and brine (50 mL), dried (Na₂SO₄), and concentrated. Chromatographic purification of the crude product (CHCl₃/MeOH 97:3) afforded 46 (0.91 g, 84%) and 49 (0.13 g, 12%) as colorless solids. Data for 46: Mp 154-156° C.; [α]_{D}²³=−25.0° (c=0.8, CH₂Cl₂). ¹H NMR (DMSO-D6): δ 7.44-7.25 (10H, m, Ar), 5.66 (1H, s, Ph-CH), 4.83 and 4.65 (2H, 2d, J_{AB}=4.2 Hz, O—CH₂—O), 4.79 (1H, d, J_{6,OH}=5.4 Hz, 6-OH) 4.77 and 4.67 (2H, 2 d, J_{AB}=10.8 Hz, Ph-CH₂), 4.56 (1H, t, J_{7,OH}=5.5 Hz, 7-OH), 4.22 (1H, dd, J_{1a,1b}=9.6, J_{1a,2}=4.2 Hz, H-1a), 3.92 (1H, br dd, J=11.4, J=6.0 Hz, H-6), 3.77-3.60 (6H, m, H-1b H-2, H-3, H-4, H-5, H-7a), 3.43 (1H, m, H-7b). ¹³C NMR (DMSO-D6): δ 143.9-131.1 (m, Ar), 104.9 (Ph-CH), 98.1 (O—CH₂—O), 91.3 (C-2), 85.0 (C-4), 82.3 (C-5), 78.9 (Ph-CH₂), 76.4 (C-6), 73.6 (C-1), 68.9 (C-3), 66.8 (C-7). HRMS Calcd for C₂₂H₂₇O₇ (M+H): 403.1757. Found: 403.1759.

4,6,7-Tri-O-benzyl 2,5-O-methylene-D-glycero-D-manno-heptitol (47)—A mixture of compound 46 (1.0 g, 2.48 mmol) and 60% NaH (3 equiv) in DMF (20 mL) was stirred in an ice bath for 20 min. A solution of benzyl bromide (0.88 ml, 7.44 mmol) in DMF (3 mL) was added, and the mixture was stirred at rt for 2 h. The reaction was quenched with ice water (40 mL) and the mixture was diluted with Et₂O (3×40 mL). The organic phase was dried (Na₂SO₄) and concentrated. The crude product was dissolved in MeOH (30 mL), p-toluenesulfonic acid (100 mg) was added, and the resulting reaction mixture was stirred for 24 h at rt. The reaction was quenched by addition of excess Et₃N (2 mL), and the solvents were removed under vacuum to give a colorless syrup which was dissolved in ethyl acetate (100 mL) and washed with water (40 mL) and brine (40 mL), dried (Na₂SO₄), and concentrated. Chromatographic purification of the crude product (hexanes/EtOAc 1:4) afforded 47 (0.91 g, 74%) as a colorless syrup. [α]_{D}²³=−15.2° (c=1.3, CH₂Cl₂). ¹H NMR (DMSO-D6): ¹H NMR (CDCl₃): δ 7.41-7.23 (15H, m, Ar), 4.84 (2H, s, O—CH₂—O), 4.79-4.54 (6H, 6 d, J_{AB}=11.5 Hz, Ph-CH₂), 4.04 (1H, ddd, J_{5,6}=2.4, J_{6,7a}=4.2, J_{6,7b}=6.6 Hz, H-6), 3.96 (1H, dd, J_{4,5}=9.0 Hz, H-5), 3.87-3.76 (2H, m, H-1a, H-b) 3.82 (1H, dd, J_{7a,7b}=10.2 Hz, H-7a), 3.74 (1H, dd, H-7b), 3.68 (2H, m, H-2, H-3), 3.58 (1H, dd, J_{3,4}=6.6 Hz, H-4). ¹³C NMR (CDCl₃): δ 138.4-127.8 (m, Ar), 93.7 (O—CH₂—O), 82.6 (C-4), 78.8 (C-6), 76.4 (C-5), 75.9 and 75.4 (C-2 and C-3), 73.9, 73.4, 72.7 (3×Ph-CH₂), 70.0 (C-7), 63.7 (C-1); HRMS Calcd for C₂₉H₃₅O₇ (M+H): 495.2383. Found: 495.2378.

4,6,7-Tri-O-benzyl-2,5-O-methylene-D-glycero-D-manno-heptitol-1,3-cyclic sulfate (48)—A mixture of 47 (0.90 g, 1.82 mmol) and Et₃N (1.0 mL, 7.28 mmol) in CH₂Cl₂ (25 mL) was stirred in an ice bath. Thionyl chloride (0.2 mL, 2.73 mmol) in CH₂Cl₂ (5 mL) was then added dropwise over 15 min, and the mixture was stirred for an additional 30 min. The mixture was poured into ice-cold water and extracted with CH₂Cl₂ (3×50 mL). The combined organic layers were washed with brine and dried over Na₂SO₄. The solvent was removed under reduced pressure and the residue was dried under high vacuum for 1 h. The diasteromeric mixture of cyclic sulfites was dissolved in a mixture of CH₃CN:CCl₄ (1:1, 50 mL) and sodium periodate (584 mg, 2.73 mmol) and RuCl₃ (20 mg) were added, followed by water (5 mL). The mixture was then stirred for 2 h at rt. The reaction mixture was filtered through Celite and washed repeatedly with ethyl acetate. The volatile solvents were removed, and the aqueous solution was extracted with EtOAc (2×50 mL). The combined organic layers were washed with saturated NaCl (50 mL), dried over Na₂SO₄, and evaporated under reduced pressure. The residue was purified by flash column chromatography (hexanes/EtOAc 4:1) to give 48 as a colorless syrup (612 mg, 61%). [α]=−1.7° (c=1.0, CH₂Cl₂). ¹H NMR (CDCl₃): δ 7.41-7.29 (15H, m, Ar), 4.87 and 4.78 (2H, 2d, J_{AB}=4.8 Hz, O—CH₂—O), 4.83 (1H, dd, J_{3,4}=7.2, J_{2,3}=10.2 Hz, H-3), 4.81-4.64 (4H, 4 d, J_{AB}=10.8 Hz, Ph-CH₂), 4.66 (1H, t, J_{1a,1b}=J_{1a,2}=11.4 Hz, H-1a), 4.54 (2H, s, Ph-CH₂), 4.52 (1H, dd, J_{1b,2}=5.4 Hz, H-1b), 4.20 (1H, td, J_{1,2}=5.4 Hz, H-2), 4.14 (1H, br t, J=6.0 Hz, H-6), 3.95-3.90 (2H, m, H-4, H-5), 3.79 (1H, dd, J_{6,7a}=5.4, J_{7a,7b}=9.6 Hz, H-7a), 3.70 (1H, dd, H-7b). ¹³C NMR (CDCl₃): δ 138.2-127.8 (m, Ar), 93.7 (O—CH₂—O), 90.8 (C-5), 78.4 (C-4), 77.9 (C-6), 75.7 (C-5), 74.9 (C-1), 73.5, 72.8, 71.9 (3×Ph-CH₂), 69.5 (C-7), 62.1 (C-2); HRMS Calcd for C₂₉H₃₃O₉S (M+H): 557.1845. Found: 557.1843.

1,3-O-Benzylidene-2,5-O-methylene-7-O-(tert-butyldimethylsilyl)-D-glycero-D-manno-heptitol-4,6-cyclic sulfate (49)—Compound 46 (200 mg, 0.49 mmol) was dissolved in MeOH (25 mL) and the solution was stirred with 10% Pd/C (100 mg) under 80 psi of H₂ for 12 h. The catalyst was removed by filtration through Celite, then evaporation of the solvent followed by purification using a short column of silica gel (CHCl₃/MeOH 9:1) gave the 1,3-O-Benzylidene-2,5-O-methylene-D-glycero-D-manno-heptitol (90 mg, 59%). A mixture of the resulting triol (50 mg, 0.16 mmol), imidazole (44 mg, 0.64 mmol), and TBDMSCl (26 mg, 0.18 mmol) in dry DMF (2 mL) was stirred at 0° C. under N₂ for 2 h. The reaction was quenched by the addition of ice-cold water (2 mL), and the reaction mixture was partitioned between Et₂O (25 mL) and H₂O (15 mL). The organic phase was washed with water (25 mL) and brine (25 mL), dried over anhydrous Na₂SO₄, and concentrated. The crude product was directly converted into the cyclic sulfate 49 by treatment with SOCl₂ and Et₃N, followed by oxidation with RuCl₃ and NaIO₄ as described for the synthesis of compound 48. Data for 49: Colorless syrup, 42 mg, yield 54% over two steps. [α]_{D}²³=−73.0° (c=2.0, CH₂Cl₂). ¹H NMR (CDCl₃): δ 7.53-7.39 (5H, m, Ar), 5.53 (1H, s, Ph-CH), 4.89 and 4.82 (2H, 2d, J_{AB}=4.2 Hz, O—CH₂—O), 4.78 (1H, dd, J_{4,5}=10.2, J_{3,4}=7.8 Hz, H-4), 4.77 (1H, ddd, J_{6,7b}=1.2, J_{6,7a}=3.0, J_{5,6}=10.2 Hz, H-6), 4.37 (1H, dd, J_{1a,2}=4.2, J_{1a,1b}=10.2 Hz, H-1a), 4.33 (1H, dd, H-5), 4.04 (1H, dd, J_{7a,7b}=12.6 Hz, H-7a), 3.94 (1H, dd, H-7b), 3.90 (1H, dd, J_{2,3}=9.0 Hz, H-3), 3.84 (1H, ddd, J_{1b,2}=10.2 Hz, H-2), 3.79 (1H, dd, H-1b), 0.95 (9H, s, TBDMS), 0.14 and 0.12 (6H, 2 s, 2×Me). ¹³C NMR (CDCl₃): δ 136.6-126.1 (m, Ar), 100.1 (Ph-CH), 93.6 (O—CH₂—O), 84.3 (C-4), 84.0 (C-6), 80.8 (C-3), 68.7 (C-1), 64.7 (C-2), 62.9 (C-5), 60.4 (C-7), 25.8 (TBDMS), −5.3 and −5.5 (2×Me). HRMS Calcd for C₂₁H₃₃O₉SSi (M+H): 489.1615. Found: 489.1617.

4-O-Benzyl-5,7-O-benzylidene-3,6-O-methylene-D-glycero-D-galacto-heptitol (50)—A mixture of AD-mix-β (3.8 g), tert-butyl alcohol (5 mL), and water (5 mL) was stirred at rt for 5 min to produce a biphasic layer. The mixture was cooled to 0° C., and the olefin 45 (1.0 g, 2.71 mmol) was added at once, and the heterogeneous slurry was stirred vigorously at 0° C. for 7 days. The reaction mixture was quenched by addition of solid sodium sulfite (4 g), stirred at rt for 30 min, extracted with ethyl acetate (3×100 mL), and the organic layer was washed with water (50 mL) and brine (50 mL), dried (Na₂SO₄), and concentrated. Chromatographic purification of the residue (CHCl₃/MeOH 97:3) afforded 50 (0.69 g, 64%) and 46 (98 mg, 9%) as colorless solids. Data for 50: Mp 208-210° C.; [α]=−12.0° (c=0.3, CH₂Cl₂). ¹H NMR (DMSO-d₆): δ 7.45-7.24 (10H, m, Ar), 5.67 (1H, s, Ph-CH), 4.83 and 4.67 (2H, 2d, J_{AB}=4.2 Hz, O—CH₂—O), 4.76 and 4.70 (2H, 2 d, J_{AB}=10.8 Hz, Ph-CH₂), 4.69 (1H, d, J_{2,OH}=6.6 Hz, 2-OH), 4.65 (1H, t, J_{1,OH}=6.0 Hz, 1-OH), 4.22 (1H, dd, J_{7a,7b}=9.6, J_{6,7a}=4.2 Hz, H-7a), 3.86 (1H, br q, J_{1,2}=J_{2,3}=7.5 Hz, H-2), 3.78-3.64 (5H, m, H-3, H-4, H-5, H-6, H-7b), 3.42 (2H, m, H-1a, H-1b). ¹³C NMR (DMSO-D6): δ 139.3-126.4 (m, Ar), 100.2 (Ph-CH), 93.0 (O—CH₂—O), 86.3 (C-5), 79.2 (C-4), 74.5 (Ph-CH₂), 73.5 (C-3), 69.2 (C-2), 68.9 (C-7), 64.3 (C-6), 62.0 (C-1). HRMS Calcd for C₂₂H₂₇O₇ (M+H): 403.1757. Found: 403.1758.

1,2,4-Tri-O-benzyl-3,6-O-methylene-D-glycero-D-galacto-heptitol (51)—Compound 51 was obtained as a colorless syrup (0.94 g, 77% yield) from 50 (1.0 g, 2.48 mmol) using the same procedure that was used to obtain 47. [α]_{D}²³=−1.7° (c=2.3, CH₂Cl₂). ¹H NMR (CDCl₃): δ 7.39-7.26 (15H, m, Ar), 4.85 and 4.66 (2H, 2d, J_{AB}=4.8 Hz, O—CH₂—O), 4.81-4.51 (6H, 6 d, J_{AB}=12.0 Hz, Ph-CH₂), 4.07 (1H, ddd, J_{2,3}=1.2, J_{1a,2}=5.4, J_{1b,2}=7.2 Hz, H-2), 3.91 (1H, dd, J_{3,4}=9.0 Hz, H-3), 3.87 (1H, m, 4, H-5, H-6), 3.69 (1H, dd, H-1b), 2.38 (1H, d, J_{5,OH}=3.6 Hz, 5-OH), 2.17 (1H, t, J_{7,OH}=6.0 Hz, 7-OH). ¹³C NMR (CDCl₃): δ 138.5-127.5 (m, Ar), 93.6 (O—CH₂—O), 81.9 (C-4), 76.1 (C-2), 75.5 (C-5), 75.0 (C-6), 74.2 (C-3), 73.6, 73.5, 72.5 (3×Ph-CH₂), 68.7 (C-1), 63.8 (C-7). HRMS Calcd for C₂₉H₃₅O₇ (M+H): 495.2383. Found: 495.2377.

1,2,4-Tri-O-benzyl-3,6-O-methylene-D-glycero-D-galacto-heptitol-5,7-cyclic sulfate (52)—Compound 52 was obtained as a colorless syrup (0.65 g, 64% yield) from 51 (0.9 g, 1.82 mmol) using the same procedure which was used to obtain 48. Colorless syrup; [α]_{D}²³=+23.2° (c=1.3, CH₂Cl₂). ¹H NMR (CDCl₃): δ 7.40-7.27 (15H, m, Ar), 4.90-4.44 (6H, 6 d, J_{AB}=11.0 Hz, Ph-CH₂), 4.88 (1H, dd, J_{4,5}=7.8, J_{5,6}=8.4 Hz, H-5), 4.83 and 4.55 (2H, 2d, J_{AB}=4.2 Hz, O—CH₂—O), 4.63 (1H, dd, J_{6,7a}=10.8, J_{7a,7b}=11.4 Hz, H-7a), 4.51 (1H, dd, J_{6,7b}=5.4 Hz, H-7b), 4.23 (1H, td, H-6), 4.15 (1H, ddd, J_{2,3}=1.8, J_{1a,2}=5.4, J_{1b,2}=7.8 Hz, H-2), 4.05 (1H, dd, J_{3,4}=10.2 Hz, H-4), 3.92 (1H, dd, H-3), 3.73 (1H, dd, J_{1a,1b}=9.6 Hz, H-1a), 3.65 (1H, dd, H-1b). ¹³C NMR (CDCl₃): δ 137.8-127.4 (m, Ar), 93.6 (O—CH₂—O), 90.9 (C-5), 77.4 (C-4), 75.0 (C-2), 74.7 (C-7), 73.9 (C-3), 73.5, 72.9, 71.9 (3×Ph-CH₂), 67.7 (C-1), 62.2 (C-6). HRMS Calcd for C₂₉H₃₃O₉S (M+H): 557.1845. Found: 557.1841.

1,4-Dideoxy-1,4-[[2S,3S,4R,5R,6R]-4,6,7-tri-O-benzyl-2,5-O-methylene-3-(sulfooxy)heptyl]-(R)-epi-sulfoniumylidine]-D-arabinitol Inner Salt (55)—The cyclic sulfate 48 (250 mg, 0.45 mmol) and the thiosugar 53 (275 mg, 0.54 mmol) were dissolved in HFIP (3 mL), and anhydrous K₂CO₃ (10 mg) was added. The mixture was stirred in a sealed tube in an oil bath (75° C.) for 7 days. The solvent was removed under reduced pressure, and the product was purified through a short silica column by eluting with EtOAc/MeOH 95:5 to yield the protected sulfonium salt 54 (351 mg) in 67% yield. To the resulting compound 54 in CH₂Cl₂ (0.5 mL) was added trifluoroacetic acid (5 mL), followed by H₂O (0.5 mL), and the mixture was stirred at rt for 2 h. The solvents were then evaporated under reduced pressure, and the residue was purified by flash column chromatography (CH₂Cl₂/MeOH 8:2) to give 55 as a colorless syrup (190 mg, 82%). [α]²: =+4.4° (c=0.9, MeOH). ¹H NMR (CD₃OD): δ 6.96-6.84 (15H, m, Ar), 4.60 (1H, d, J_{AB}=10.2 Hz, Ph-CH₂), 4.51 and 4.37 (2H, 2d, J_{AB}=4.2 Hz, O—CH₂—O), 4.26 (2H, 2d, J_{AB}=12.0 Hz, Ph-CH₂), 4.20 (1H, br dd, J=2.4 Hz, H-2), 4.12 (1H, dd, J_{2,3}=7.8, J_{3,4}=6.6 Hz, H-3′), 4.07 (1H, d, J_{AB}=12.0 Hz, Ph-CH₂), 4.06 (2H, br s, Ph-CH₂), 4.01 (1H, br d, J=1.8 Hz, H-3), 3.97 (1H, td, J_{1′a,2}=7.8, J_{1′b,2′}3.6 Hz, H-2′), 3.69 (1H, dd, J_{1′a,1′b}=13.2 Hz, H-1′a), 3.61-3.57 (4H, m, H-1′b, H-4, H-5a, H-6′), 3.53-3.43 (2H, m, H-5b, H-5′), 3.47 (1H, dd, J_{1a,1b}=12.0, J_{1a,2}=1.8, H-1a), 3.45 (1H, dd, J_{4′,5′}=7.8 Hz, H-4′), 3.39 (1H, dd, J_{1b,2}=3.6, H-1b), 3.34 (1H, dd, J_{7′a,7′b}=10.8, J_{7′a,6′}=3.6 Hz, H-7a) 3.24 (1H, dd, J_{7b,6}′=6.0 Hz, H-7′b). ¹³C NMR (CD₃OD): δ 137.9-126.8 (m, Ar), 93.1 (O—CH₂—O), 80.8 (C-3′), 80.6 (C-4′), 78.1 (C-3), 78.0 (C-4), 77.1 (C-2), 76.6 (C-5′), 73.4, 72.5 and 71.6 (3×CH₂Ph), 71.5 (C-6′), 70.8 (C-2′), 68.9 (C-7′), 59.1 (C-5), 49.5 (C-1), 49.2 (C-1′). HRMS Calcd for C₃₄H₄₃O₁₂S₂ (M+H): 707.2195. Found: 707.2195.

1,4-Dideoxy-1,4-[[2S,3S,4R,5R,6R]-2,3,4,5,6,7-hexahydroxy-heptyl]-(R)-epi-sulfoniumylidine]-D-arabinitol methyl sulfate (56)—To a solution of compound 55 (150 mg, 0.21 mmol) in CH₂Cl₂ (10 mL) at −78° C. was added 1.0 M BCl₃ (2 mL) in CH₂Cl₂. The mixture was then warmed to rt over a period of 20 min and stirred for 12 h. MeOH was added to quench the reaction mixture and all the volatile components were removed under reduced pressure. The residue was dissolved in water (5 mL) and washed with CH₂Cl₂ (3×5 mL). The water layer was evaporated to give a crude product which was purified by reverse-phase HPLC [MeCN—H₂O (4:96, v/v) to yield compound 56 (54 mg, 74%) as a colorless syrup. [α]_{D}²³=−4.0° (c=0.8, MeOH). ¹H NMR (CD₃OD): δ 4.62 (1H, br d, J=2.4 Hz, H—H-2), 4.37 (1H, br s, H-3), 4.17 (1H, td, J_{1′a,2}=3.6, J_{1′b,2}=J_{2′,3′}=8.4 Hz, H-2′), 4.05 (1H, dd, J_{4,5a}=4.8, J_{5a,5b}=10.8 Hz, H-5a), 4.02 (1H, dd, J_{4,5b}=9.6 Hz, H-4), 3.93 (1H, dd, H-5b), 3.94 (1H, dd, J_{1′a,2′}=3.6, J_{1′b,1′b}=12.6 Hz, H-1a′), 3.88 (1H, dd, J_{3′,4′}=2.4, J_{4′,5′}=7.2 Hz, H-4′), 3.86 (2H, d like, J=2.4 Hz, H-1a, H-1b), 3.85 (1H, dd, H-3′), 3.83 (1H, d like, J=7.8 Hz, H-6′), 3.80 (1H, br d, J=9.6 Hz, H-7′a), 3.75 (1H, dd, 3.71 (1H, d like, J=6.6 Hz, H-5′), 3.68 (3H, s, CH₃OSO₃), 3.67 (1H, m, H-7′b). ¹³C NMR (CD₃OD): δ 79.5 (C-3), 79.4 (C-2), 74.8 (C-6′), 74.0 (C-3′), 73.7 (C-4), 73.1 (C-5′), 71.9 (C-4′), 69.4 (C-2′), 64.4 (C-7′), 61.1 (C-5), 55.2 (CH₃OSO₃) δ2.7 (C-1′), 51.9 (C-1). HRMS Calcd for C₁₃H₂₈O₁₂S₂ (M-CH₃OSO₃): 345.1219. Found: 345.1218.

1,4-Dideoxy-1,4-[[2S,3S,4R,5R,6S]-2,3,4,5,6,7-hexahydroxy-heptyl]-(R)-epi-sulfoniumylidine]-D-arabinitol methyl sulfate (58)—The cyclic sulfate 52 (250 mg, 0.45 mmol) and the thiosugar 53 (275 mg, 0.54 mmol) were dissolved in HFIP (3 mL), and anhydrous K₂CO₃ (10 mg) was added. The mixture was stirred in a sealed tube in an oil bath (75° C.) for 7 days. The solvent was removed under reduced pressure, and the product was purified through a short silica column by eluting with EtOAc/MeOH 95:5 to yield the protected sulfonium salt 57 (325 mg, 61%). To a solution of the protected compound 57 (200 mg, 0.19 mmol) in CH₂Cl₂ (10 mL) at −78° C. was added 1.0 M BCl₃ (3 mL) in CH₂Cl₂. The mixture was then warmed to rt over a period of 20 min and stirred for 12 h. MeOH was added to quench the reaction mixture and all the volatile components were removed under reduced pressure. The residue was dissolved in water (5 mL) and washed with CH₂Cl₂ (3×5 mL). The water layer was evaporated to give a crude product that was purified by reverse-phase HPLC [MeCN—H₂O (4:96, v/v) to yield compound 58 (40 mg, 61%) as a colorless syrup. [α]_{D}²³=+10.0° (c=0.6, MeOH). ¹H NMR (CD₃OD): δ 4.62 (1H, ddd, J_{1a,2}=3.0, J_{1b,2}=J_{2,3}=2.4 Hz, H-2), 4.37 (1H, dd. J_{3,4}=1.2 Hz, H-3), 4.18 (1H, td, =3.6, J_{1′b,2}=J_{2′,3′}=8.4 Hz, H-2′), 4.05 (1H, dd, J_{4,5a}=4.8, J_{5a,5b}=10.8 Hz, H-5a), 4.01 (1H, br dd, J_{4,5b}=9.0 Hz, H-4), 3.94 (1H, dd, J_{1a,1b}=13.2 Hz, H-1′a), 3.93 (1H, m, H-6′), 3.87 (2H, br d, J=3.0 Hz, H-1a, H-1b), 3.85 (1H, dd, J_{3′,4′}=1.2 Hz, H-3′), 3.84 (1H, br d, J_{4′,5′}=7.8 Hz, H-5′), 3.76 (1H, dd, H-1′b), 3.69 (3H, s, CH₃OSO₃), 3.66 (2H, br d, J=6.6 Hz, H-7′a, H-7′b), 3.65 (1H, dd, H-4′). ¹³C NMR (CD₃OD): δ 79.5 (C-3), 79.4 (C-2), 73.7 (C-4), 73.6 (C-5′), 71.7 (C-6′), 71.2 (C-4′), 70.2 (C-3′), 69.7 (C-2′), 64.9 (C-7′), 61.1 (C-5), 55.2 (CH₃OSO₃) δ2.7 (C-1′), 51.9 (C-1). HRMS Calcd for C₁₃H₂₈O₁₂S₂ (M-CH₃OSO₃): 345.1219. Found: 345.1216.

### 9.4 Synthesis of Kotalanol 20

5,7-Di-O-benzylidene-2,4,6-tri-O-p-methoxybenzyl-D-perseitol (60) and 1,3-Di-O-benzylidene-2,4,6-tri-O-p-methoxybenzyl-D-perseitol (61)—A mixture of compound 59³⁹ (8.50 g, 21.89 mmol) and 60% NaH (4 equiv) in DMF (90 mL) was stirred in an ice bath for 20 min. A solution of p-methoxybenzyl chloride (12.2 ml, 87.55 mmol) in DMF (20 mL) was added, and the mixture was stirred at rt for 2 h. The reaction was quenched with ice water (150 mL) and the mixture was diluted with Et₂O (3×150 mL). The organic phase was dried (Na₂SO₄) and concentrated. The crude product was dissolved in MeOH (100 mL), p-toluenesulfonic acid (2.0 g) was added, and the resulting reaction mixture was stirred for 30 min at rt. The reaction was quenched by addition of excess Et₃N (˜20 mL), and the solvents were removed under vacuum to give a colorless syrup which was dissolved in ethyl acetate (500 mL) and washed with water (100 mL) and brine (100 mL), dried (Na₂SO₄), and concentrated. Chromatographic purification of the crude product (hexanes/EtOAc 3:7) afforded 60 (4.0 g, 44%) and 61 (3.1 g, 34%) (yield was calculated based on recovered 1,3:5,7-di-O-benzylidene-2,4,6-tri-O-p-methoxybenzyl-D-perseitol, 6.0 g).

Data for 60: Pale yellow syrup, [α]_{D}²³=+19.0° (c=1.1, CHCl₃). ¹H NMR (CDCl₃+D₂O): δ 7.47-6.86 (17H, m, Ar), 5.46 (1H, s, Ph-CH), 4.70-4.41 (6H, 6 d, J_{AB}=11.4 Hz, Ph-CH₂), 4.44 (1H, dd, J_{7a,7b}=10.2, J_{7a,6}=4.2 Hz, H-7a), 4.14 (1H, dd, J_{5,6}=9.6, J_{4,5}=1.8 Hz, H-5), 4.12 (1H, dd, J_{3,4}=8.4, J_{2,3}=1.8 Hz, H-3), 3.99 (1H, dd, H-4), 3.96 (1H, dd, J_{1a,1b}=12.6, J_{1a,2}=4.2 Hz, H-1a), 3.93 (1H, td, J_{6,7b}=10.2 Hz, H-6), 3.81 (9H, br s, 3×OMe), 3.80 (1H, dd, J_{1b,2}=1.8 Hz, H-1b), 3.75 (1H, td, H-2), 3.66 (1H, dd, H-7b). ¹³C NMR (CDCl₃-D₂O): δ 159.8, 159.4 and 159.1 (Ar), 137.6 and 129.7-113.7 (m, Ar), 101.6 (Ph-CH), 79.9 (C-5), 76.3 (C-2), 75.8 (C-4), 72.6, 71.4, and 71.2 (3×Ph-CH₂), 71.4 (C-3), 69.7 (C-7), 68.1 (C-6), 63.1 (C-1), 55.3 (3×OMe). HRMS Calcd for C₃₈H₄₅O₁₀ (M+H): 661.3012. Found: 661.3003.

Data for 61: Pale yellow syrup, [α]_{D}²³=+22.5° (c=0.8, CHCl₃). ¹H NMR (CDCl₃): δ 7.52-6.80 (17H, m, Ar), 5.60 (1H, s, Ph-CH), 4.84-4.28 (6H, 6 d, J_{AB}=11.4 Hz, Ph-CH₂), 4.63 (1H, dd, J_{1a,1b}=12.6, J_{1a,2}=1.2 Hz, H-1a), 4.26 (1H, dd, J_{3,4}=9.0, J_{4,5}=1.2 Hz, H-4), 4.15 (1H, dd, J_{2,3}=1.2 Hz, H-3), 4.09 (1H, br d, J_{5,6}=8.4 Hz, H-5), 3.96 (1H, dd. J_{1b,2}=1.2 Hz, H-1b), 3.91-3.90 (2H, m, H-7a, H-7b), 3.82, 3.80 and 3.77 (9H, 3 s, 3×OMe), 3.62 (1H, br d, H-2), 3.56 (1H, ddd, J_{6,7a}=J_{6,7b}=4.2 Hz, H-6). ¹³C NMR (CDCl₃): δ 159.3, 159.3 and 159.1 (Ar), 137.9 and 130.2-113.8 (m, Ar), 101.5 (Ph-CH), 78.3 (C-3), 78.1 (C-6), 74.6 (C-4), 73.6, 70.7, and 69.7 (3×Ph-CH₂), 70.3 (C-5), 69.3 (C-2), 67.3 (C-1), 61.4 (C-7), 55.4, 55.3 (3×OMe). HRMS Calcd for C₃₈H₄₅O₁₀ (M+H): 661.3012. Found: 661.3005.

1,3-O-Benzylidene-2,4,6-tri-O-p-methoxybenzyl-D-perseitol-5,7-cyclic sulfate (62)—Compound 62 was obtained as a colorless foam (2.5 g, 77% yield) from 61 (3.0 g, 4.54 mmol) using the same procedure as used to obtain 48. Data for 62: [α]_{D}²³=+5.8° (c=0.5, CHCl₃). ¹H NMR (CDCl₃): δ 7.59-6.84 (17H, m, Ar), 5.67 (1H, s, Ph-CH), 5.16 (1H, dd, J_{5,6}=9.6, J_{4,5}=1.2 Hz, H-5), 4.86 (1H, d, J_{AB}=11.4 Hz, Ph-CH₂), 4.67 (1H, dd, J_{1a,1b}=13.2, J_{1a,2}=1.2 Hz, H-1a), 4.48-4.45 (2H, 2 d, J_{AB}=11.4 Hz, Ph-CH₂), 4.45-4.43 (3H, m, H-4, H-7a, H-7b), 4.35 s, Ph-CH₂), 4.28 (1H, d, J_{AB}=11.4 Hz, Ph-CH₂), 4.20 (1H, dd, J_{3,4}=10.2, J_{2,3}=1.8 Hz, H-3), 4.13 (1H, td, J_{6,7a}=J_{6,7b}=7.2 Hz, H-6), 3.99 (1H, dd, J_{1b,2}=1.2 Hz, H-1b), 3.83, 3.81 and 3.80 (9H, 3 s, 3×OMe), 3.64 (1H, br d, H-2). ¹³C NMR (CDCl₃): δ NMR (CDCl₃): 159.8, 159.3, 137.5, 129.9-113.8 (m, Ar), 101.1 (Ph-CH), 84.2 (C-5), 76.3 (C-3), 73.9, 72.1, and 69.7 (3×Ph-CH₂), 73.1 (C-4), 71.9 (C-7), 68.8 (C-2), 67.0 (C-1), 66.7 (C-6), 55.4, 55.3 (3×OMe). HRMS Calcd for C₃₈H₄₃O₁₂S (M+Na): 745.2294. Found: 745.2277.

2,3,5-Tri-O-p-methoxybenzyl-1,4-dideoxy-1,4-[[2S,3S,4R,5R,6S]-5,7-benzylidene-2,4,6-tri-O-p-methoxybenzyl-3-(sulfooxy)heptyl]-(R)-epi-sulfoniumylidine]-D-arabinitol Inner Salt (63)—Compound 63 was obtained as a colorless syrup (238 mg, 69% yield) by reacting compounds 62 (200 mg, 0.28 mmol) and 53 (171 mg, 0.34 mmol) using the same procedure as used to obtain 54. [α]_{D}²³=+5.4° (c=0.4, acetone). ¹H NMR (acetone-d₆): δ 7.71-6.75 (29H, m, Ar), 5.78 (1H, s, Ph-CH), 4.96 (1H, br d, J_{3′,4′}=9.6 Hz, H-3′), 4.85-4.15 (12H, Ph-CH₂), 4.67 (1H, br d, J_{7′a,7′b}=12.6 Hz, H-7a), 4.66 (1H, ddd, J_{1a,2}=2.4, J_{1′b,2}=3.6, J_{2,3}=3.0 Hz. H-2), 4.61 (1H, m, H-5′), 4.48 (1H, br d, H-3), 4.39 (1H, dd, J_{1′a,1′b}=13.8, J_{1′a,2}=4.2 Hz, H-1′a), 4.29 (1H, dd, J_{1′b,2}=2.4 Hz, H-1′b), 4.27 (1H, ddd, J_{2′,3′}=1.8 Hz, H-2′), 4.25 (1H, br s, H-4′), 4.10 (1H, dd, J_{1a,1b}=13.8 Hz, H-1a), 4.04 (1H, br d, H-7′b), 3.92 (1H, dd-like, J_{5a,4}=7.8, J_{5b,4}=7.2 Hz, H-4), 3.89 (1H, dd, H-1b), 3.81-3.72 (18H, 6s, OMe), 3.74 (1H, m, H-6′), 3.60 (1H, dd, J_{5a,5b}=10.2 Hz, H-5a), 3.54 (1H, dd, H-5b). ¹³C NMR (acetone-d₆): δ 159.8-159.0, 139.6, 129.8-126.6, 113.8-113.4 (m, Ar), 100.4 (Ph-CH), 83.4 (C-3), 81.7 (C-2), 76.7 (C-5′), 74.4 (C-4′), 74.2 (C-2′), 73.5 (C-3′), 72.7-69.3 (6×Ph-CH₂), 70.6 (C-6′), 66.9 (C-7′), 66.3 (C-5), 64.5 (C-4), 54.7-54.6 (6×OMe), 49.6 (C-1′), 47.8 (C-1). HRMS Calcd for C₆₇H₇₇O₁₈S₂ (M+H): 1233.4551. Found: 1233.4561.

1,4-Dideoxy-1,4-[[2S,3S,4R,5R,6S]-2,4,5,6,7-pentahydroxy-3-(sulfooxy)heptyl]-(R)-epi-sulfoniumylidine]-D-arabinitol Inner Salt (20). Compound 63 (100 mg, 0.08 mmol) in CH₂Cl₂ (0.5 mL) was added trifluoroacetic acid (5 mL), followed by H₂O (0.5 mL), and the mixture was stirred at rt for 2 h. The solvents were then evaporated under reduced pressure, and the residue was dissolved in water (5 mL) and washed with CH₂Cl₂ (3×5 mL). The water layer was evaporated to give a crude product that was purified on silica gel column by eluting with EtOAc/MeOH/H₂O 7:3:1 (v/v) to give compound 20 in 93% yield (32 mg) as a colorless solid. [α]=_{D}²³=+7.0° (c=0.6, H₂O). ¹H NMR (pyridine-d₅) (coupling constant values are determined by D₂O addition): δ 5.64 (1H, dd, J_{2′,3′}=8.4, J_{3′,4′}=1.2 Hz, H-3′), 5.24 (1H, ddd, J_{1′a,2′}=J_{1b,2′}=4.2 Hz, H-2′), 5.15 (1H, br s, H-3), 5.12 (1H, dd, J_{4′,5′}=9.6 Hz, H-4′), 5.07 (1H, dd-like. J_{1a,2}=1.8, J_{1b,2}=3.6 Hz, H-2), 4.93 (1H, dd, J_{1′a,1′b}=13.2 Hz, H-1′a), 4.88 (1H, ddd, =1.8, J_{6′,7′a}=5.4, J_{6′,7′b}=4.2 Hz, H-6′), 4.86 (1H, dd, H-5′), 4.65 (1H, dd, H-1′b), 4.62 (1H, br t, J_{4,5a}=J_{4,5b}=10.2 Hz, H-4), 4.51 (2H, dd-like, J=7.8 Hz, H-5a, H-5b), 4.40 (1H, dd, J_{7′a,7b}=10.8 Hz, H-7′a), 4.31 (2H, dd-like, J_{1a,1b}=13.2 Hz, H-1a, H-1b), 4.24 (1H, dd, H-7′b). ¹³C NMR (pyridine-d₅): δ 79.4 (C-3), 78.1 (C-2), 77.9 (C-3′), 72.6 (C-6′), 72.2 (C-4), 71.3 (C-5′), 70.5 (C-4′), 67.4 (C-2′), 65.4 (C-7′), 60.0 (C-5), 53.8 (C-1′), 50.1 (C-1). HRMS Calcd for C₁₂H₂₅O₁₂S₂ (M+Na): 447.0606. Found: 447.0596.

### 9.5 Synthesis of Selenium and Nitrogen Analogues of Kotalanol

1,4-Dideoxy-1,4-[[2S,3S,4R,5R,6S]-2,4,5,6,7-pentahydroxy-3-(sulfooxy)heptyl]-(R/S)-epi-selenoniumylidine]-D-arabinitol Inner Salt (67 and 68)—The cyclic sulfate 62 (712 mg, 0.99 mmol) and the selenoarabinitol 64 (502 mg, 0.90 mmol) were dissolved in HFIP (3 mL), and anhydrous K₂CO₃ (20 mg) was added. The mixture was stirred in a sealed tube in an oil bath (75° C.) for 5 days. The solvent was removed under reduced pressure, and the product was purified through a short silica column by eluting with EtOAc/MeOH 95:5 to yield the protected selenonium salts 65 (454 mg, 40%) and 66 (300 mg, 26%). To a solution of the protected compound 65 (370 mg, 0.29 mmol) in CH₂Cl₂ (0.5 mL) was added trifluoroacetic acid (5 mL), followed by H₂O (1.0 mL), and the mixture was stirred at rt for 2 h. The solvents were then evaporated under reduced pressure, and the residue was dissolved in water (5 mL) and washed with CH₂Cl₂ (3×5 mL). The water layer was evaporated to give a crude product that was purified on silica gel column by eluting with EtOAc/MeOH/H₂O 7:3:1 (v/v) to give compound 67 in 89% yield (122 mg) as a colorless foam. Similarly, compound 68 was obtained from 66 (175 mg, 0.14 mmol) in 83% yield (53 mg) as a colorless foam.

Data for 67: [α]_{D}²³=+16.8° (c=1.2, H₂O). ¹H NMR (D₂O): δ 4.74 (1H, q, 0.1=3.6 Hz, H-2), 4.57 (1H, dd, J_{3′,4′}=0.6, J_{2′,3′}=7.8 Hz, H-3′), 4.45 (1H, dd, J_{3,4}=3.0, J_{2,3}=3.6 Hz, H-3) 4.38 (1H, ddd, J_{1′a,2′}=3.6, J_{1′b,2′}=6.6 Hz, H-2′), 4.12 (1H, ddd, J_{4,5a}=4.8, J_{4,5b}=8.4 Hz, H-4), 4.05 (1H, dd, J_{1′a,1′b}=12.6 Hz, H-1′a), 4.02 (1H, dd, J_{5a,5b}=12.6 Hz, H-5a), 3.91-3.90 (4H, m, H-1′b), H-4′, H-6′, H-5b), 3.74-3.73 (3H, m, H-1a, H-1b, H-5′), 3.62-3.60 (2H, m, H-7′a, H-7′b). ¹³C NMR (D₂O): δ 78.7 (C-3′), 78.4 (C-3), 77.5 (C-2), 69.9 (C-6′), 69.8 (C-4), 68.7 (C-5′), 68.0 (C-4′), 66.1 (C-2′), 63.2 (C-7′), 59.2 (C-5), 48.9 (C-1′), 44.8 (C-1). HRMS Calcd for C₁₂H₂₅O₁₂SSe (M+H): 473.0231. Found: 473.0229.

Data for 68: [α]_{D}²³==+106.6° (c=0.5, H₂O). ¹H NMR (D₂O): δ 4.69 (1H, q, J=3.6 Hz, H-2), 4.57 (1H, dd, J_{3′,4′}=0.6, J_{2′,3′}=7.8 Hz, H-3′), 4.49 (1H, t, J=3.6 Hz, H-3), 4.36 (1H, td, J_{1′b,2′}=4.2, J_{1′a,2′}=7.8 Hz, H-2′), 4.20 (1H, m, H-4), 4.15 (1H, dd, J_{4,5a}=6.0, J_{5a,5b}=12.6 Hz, H-5a), 4.04 (1H, m, H-5b), 4.01 (1H, dd, H-1′a), 3.92-3.89 (2H, m, H-4′, H-6′), 3.86 (1H, dd, 12.6 Hz, H-1′b), 3.78 (1H, dd, J_{1a,1b}=12.6 Hz, H-1a), 3.73 (1H, dd, J_{4′,5′}=9.6 Hz, H-5′), 3.63-3.60 (2H, m, H-7′a, H-7′b), 3.56 (1H, dd, H-1b). ¹³C NMR (D₂O): δ 79.0 (C-3′), 78.4 (C-2), 78.1 (C-3), 69.9 (C-6′), 68.7 (C-5′), 68.1 (C-4′), 66.0 (C-2′), 63.9 (C-4), 63.2 (C-7′), 58.0 (C-5), 42.4 (C-1), 41.4 (C-1′). HRMS Calcd for C₁₂H₂₅O₁₂SSe (M+H): 473.0231. Found: 473.0229.

7′-((1,4-Dideoxy-1,4-imino-D-arabinitol)-4-N-ammonium)-7′-deoxy-D-perseitol-5-sulfate (72)—The cyclic sulfate 62 (442 mg, 0.61 mmol) and the iminoarabinitol 71 (250 mg, 0.51 mmol) were dissolved in acetone (3 mL), and anhydrous K₂CO₃ (20 mg) was added. The mixture was stirred in a sealed tube in an oil bath (60° C.) for 5 days. The solvent was removed under reduced pressure, and the product was purified through a short silica column by eluting with EtOAc/MeOH 95:5 to yield the protected ammonium salt which was then deprotected using the same procedure used for 67 to give compound 72 (108 mg, 52% yield, for two steps) as a colorless foam. [α]_{D}²³=+6.4° (c=1.4, H₂O). ¹H NMR (D₂O, pH=8 by adding K₂CO₃): δ 4.62 (1H, d, =4.8 Hz, H-3′), 4.06-4.03 (2H, m, H-2′. H-2), 3.88 (1H, td, J_{5′,6′}=1.2, J_{6′,7′a}=J_{6′,7′b}=6.0 Hz, H-6′), 3.85-3.83 (2H, m, H-3, H-4′), 3.66 (1H, dd, J_{4′,5′}=9.6 Hz, H-5′), 3.65-3.59 (4H, m, H-5a, H-5b, H-7′a, H-7′b), 3.21 (1H, dd, J_{1′a,2′}=6.6, J_{1′a,1′b}=12.6 Hz, H-1′a), 3.06 (1H, br d, J_{1a,1b}=11.4 Hz, H-1a), 2.78 (1H, dd, J_{1b,2}=5.4 Hz, H-1b), 2.52 (1H, q, J=4.8 Hz, H-4), 2.45 (1H, dd, J_{1′b,2′}=6.6 Hz, H-1′b). ¹³C NMR (D₂O, pH=8 by adding K₂CO₃): 79.2 (C-3′), 78.5 (C-3), 75.6 (C-2), 72.3 (C-4), 70.7 (C-2′), 69.8 (C-6′), 68.8 (C-4′, C-5′), 63.2 (C-7′), 60.6 (C-5), 59.4 (C-1), 56.6 (C-1′). HRMS Calcd for C₁₂H₂₆NO₁₂S (M+H): 408.1175. Found: 408.1170.

9.6 Preparation of de-O-sulfonated Analogues of Kotalanol

1,4-Dideoxy-1,4-[[2S,3S,4R,5R,6S]-2,3,4,5,6,7-hexahydroxy-heptyl]-(R/S)-epi-selenoniumylidine]-D-arabinitol chloride (69 and 70)—Compound 67 (25 mg, 0.05 mmol) was stirred in 5% methanolic HCl (3 mL) at rt for 3.5 h. Solvent was evaporated followed by treatment with Amberlyst A-26 resin (20 mg, chloride form) gave 69 as a colorless syrup in quantitative yield (21 mg). Similarly, compound 70 (13 mg, quantitative) was obtained from 68 (15 mg, 0.03 mmol) as a colorless syrup.

Data for 69: [α]_{D}²³=+15.0° (c=0.4, H₂O). ¹H NMR (D₂O): δ 4.81 (1H, q, J=3.6 Hz, H-2), 4.50 (1H, t, J=3.6 Hz, H-3), 4.24 (1H, td, J_{1′a,2′}=4.2, J_{2′,3′}=J_{1′b,2′}=7.8 Hz, H-2′), 4.20 (1H, ddd, J_{4,5a}=4.8, J_{4,5b}=8.4 Hz, H-4), 4.10 (1H, dd, J_{5a,5b}=12.6 Hz, H-5a), 3.97 (1H, dd, J_{1′a,1′b}12.0 Hz, H-1′a), 3.96 (1H, m, H-6′), 3.94 (1H, dd, H-5b), 3.89 (1H, d, H-3′), 3.86 (1H, m, H-4′), 3.84 (1H, dd, H-1′b), 3.82 (1H, dd, J_{1a,1b}=12.0 Hz, H-1a), 3.79 (1H, dd, H-1b), 3.66 (2H, d. J=6.6 Hz, H-7′a, H-7b), 3.64 (1H, dd, J=0.6, J=9.0 Hz, H-5′). ¹³C NMR (D₂O): δ 78.2 (C-3), 77.6 (C-2), 72.0 (C-3′), 69.9 (C-6′), 69.5 (C-4), 69.1 (C-5′), 68.1 (C-4′), 67.5 (C-2′), 63.1 (C-7′), 59.3 (C-5), 48.0 (C-1′), 45.2 (C-1). HRMS Calcd for C₁₂H₂₅ClO₉SSe (M-Cl): 393.0663. Found: 393.0658.

Data for 70: [α]_{D}²³=+96.6° (c=0.6, H₂O). ¹H NMR (D₂O): δ 4.74 (1H, q, J=4.2 Hz, H-2), 4.50 (1H, dd, J_{3,4}=3.6 Hz, H-3), 4.28-4.21 (3H, m, H-4, H-5a, H-2′), 4.07 (1H, dd, J_{4,5b}=11.4, J_{5a,5b}=13.8 Hz, H-5b), 3.98 (1H, dd, J_{1′a,2′}=4.2, J_{1′a,1′}=12.0 Hz, H-1′a), 3.95 (1H, td, J_{5′,6′}=1.2, J_{6′,7′a}=6.0 Hz, H-6′), 3.91 (1H, dd, J_{2′,3′}=7.8, J_{4′,3′}=0.6 Hz, H-3′), 3.88-3.85 (2H, m, H-4′, H-1a), 3.84 (1H, dd, J_{2′,1′b}=8.4 Hz, H-1′b), 3.67 (2H, d, J=6.6 Hz, H-7a, H-7b), 3.64 (1H, dd, J_{4′,5′}=5.4 Hz, H-5′), 3.63 (1H, dd, J_{1b,2}=4.2, J_{1a,1b}=13.2 Hz, H-1b). ¹³C NMR (D₂O): δ 78.2 (C-2), 78.1 (C-3), 71.9 (C-3′), 69.9 (C-6′), 69.1 (C-5′), 68.0 (C-4′), 67.1 (C-2′), 63.9 (C-4), 63.1 (C-7′), 58.0 (C-5), 42.1 (C-1′), 41.0 (C-1). HRMS Calcd for C₁₂H₂₅ClO₉SSe (M-Cl): 393.0663. Found: 393.0658.

7′-((1,4-Dideoxy-1,4-imino-D-arabinitol)-4-N-ammonium)-7′-deoxy-D-perseitol chloride (73)—Compound 73 was obtained as a colorless foam (21 mg, quantitative) from compound 72 (26 mg, 0.06 mmol) using the same procedure as described to obtain 69. [α]_{D}²³=+6.6° (c=0.75, H₂O). ¹H NMR (D₂O): δ 4.3 (1H, m, H-2), 4.09 (1H, br t, J_{2′,1′b}=J_{2′,3′}=9.0 Hz, H-2′), 4.06 (1H, br s, H-3), 3.95 (1H, dd, J_{5a,4}=4.8, J_{5a,5b}=12.6 Hz, H-5a), 3.92-3.88 (2H, m. H-5b, H-6′), 3.83-3.78 (3H, m, H1a, H-1′a, H-4′), 3.72 (1H, dd. J_{3′,4′}=0.6 Hz, H-3′), 3.61-3.57 (5H, m, H-1b, H-4, H-5′, H-7′a, H-7′b), 3.29 (1H, dd, J_{1′b,1′a}=12.6 Hz, H-1′b). ¹³C NMR (D₂O): 75.8 (C-3, C-4), 73.9 (C-2), 71.0 (C-3′), 70.0 (C-6′), 69.0 (C-5′), 67.8 (C-4′), 66.6 (C-2′), 63.2 (C-7′), 60.8 (C-1), 60.3 (C-1′), 58.4 (C-5). HRMS Calcd for C₁₂H₂₆NO₉ (M-Cl) 328.1607. Found: 328.1602.

1,4-Dideoxy-1,4-[[2S,3S,4R,5S,6S]-2,3,4,5,6,7-hexahydroxy-heptyl]-(R)-epi-sulfnoniumylidine]-D-arabinitol chloride (74)—Compound 74 was obtained as a colorless foam (18 mg, quantitative) from compound 17 (22 mg, 0.06 mmol) using the same procedure as described to obtain 69. [α]_{D}²³=+10.5° (c=0.5, H₂O). ¹H NMR (D₂O): δ 4.77 (1H, q, J=3.6 Hz, H-2), 4.47 (1H, dd, J=3.6 Hz, H-3), 4.27 (1H, ddd, J_{1′a,2′}=3.0, J_{2′3′}=7.2, J_{2′,1′b}9.6 Hz, H-2′), 4.16 (1H, dd, J_{4,5a}=4.8, J_{5a,5b}=11.4 Hz, H-5a), 4.13 (1H, ddd, J_{4,5b}=7.2 Hz, H-4), 4.00 (1H, t, J=3.0 Hz, H-4′), 3.97 (1H, dd, H-5b), 3.95 (1H, dd, J_{1′a,1′b}=13.8 Hz, H-1′a), 3.94 (1H, dd, J_{1a,1b}=13.2 Hz, H-1a), 3.91 (1H, dd, H-1b), 3.84 (1H, dd, J_{2′,3′}=7.2 Hz, H-3′), 3.82 (1H, dd, H-1′b), 3.80 (1H, dd, J_{6′,7 a}=2.4, J_{7′a,7′b}=12.0 Hz, H-7′a), 3.76 (1H, ddd, J_{6′,7′b}=6.0, J_{5′,6′}=7.8, Hz H-6′), 3.73 (1H, dd, H-5′), 3.67 (1H, dd, H-7′b). ¹³C NMR (D₂O): δ 77.5 (C-3), 76.9 (C-2), 74.6 (C-3′), 72.5 (C-5′), 71.0 (C-6′), 69.9 (C-4), 68.1 (C-4′), 67.4 (C-2′), 62.4 (C-7′), 59.1 (C-5), 49.7 (C-1′), 48.2 (C-1). HRMS Calcd for C₁₂H₂₅O₉SCl (M-Cl): 345.1219. Found: 345.1210.

1,4-Dideoxy-1,4-[[2S,3S,4R,5S,6R]-2,3,4,5,6,7-hexahydroxy-heptyl]-(R)-epi-sulfnoniumylidine]-D-arabinitol chloride (75)—Compound 75 was obtained as a colorless foam (20 mg, quantitative) from compound 18 (24 mg, 0.06 mmol) using the same procedure as described to obtain 69. [α]_{D}²³=+8.3° (c=0.4, H₂O). ¹H NMR (D₂O): δ 4.78 (1H, q, J=3.6 Hz, H-2), 4.47 (1H, t, J=3.0 Hz, H-3), 4.29 (1H, ddd-like, H-2′), 4.16 (1H, dd, J_{4,5a}=4.8, J_{5a,5b}=11.4 Hz, H-5a), 4.13 (1H, ddd, J_{3,4}=1.8, J_{4,5b}=6.6 Hz, H-4), 3.99 (1H, dd, H-5b), 3.96 (2H, m, H-1′a, H-4′), 3.94 (1H, dd, J_{1a,1b}=13.2 Hz, H-1a), 3.90 (1H, dd, H-1b), 3.83 (1H, ddd, J_{5′,6′}=3.0, J_{6′,7′a}=4.8, J_{6′,7′b}=7.2 Hz, H-6′), 3.80 (2H, m, H-1′b, H-3′), 3.77 (1H, dd, J_{4′,5′}=6.6 Hz, H-5′), 3.72 (1H, dd, J_{7′a,7′b}=11.4 Hz, H-7′a), 3.67 (1H, dd, H-7′b. ¹³C NMR (D₂O): δ 77.5 (C-3), 76.9 (C-2), 72.7 (C-3′), 71.6 (C-5′), 70.9 (C-6′), 70.0 (C-4), 69.5 (C-4′), 67.4 (C-2′), 62.8 (C-7′), 59.2 (C-5), 50.0 (C-1′), 48.2 (C-1). HRMS Calcd for C₁₂H₂₅O₉SCl (M-Cl): 345.1219. Found: 345.1213.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### REFERENCES

1. Bertozzi, C. R.; Kiessling, L. L. Science 2001, 291, 2351; Moremen, K. W.; Trimble, R. B.; Herscovics, A. Glycobiology 1994, 4, 113.
2. Mehta, A.; Zitzmann, N.; Rudd, D. M.; Block, T. M.; Dwek, R. A. FEBS Lett. 1998, 430, 17. Depracter, C. M.; Gerwig, G. J.; Bause, E.; Nuytinck, L. K.; Vliegenthart, J. F. G.; Breuer, W.; Kamarling, J. P.; Espeel, M. F.; Martin, J. J. R.; De Paepe, A. M.; Chan, N. W. C.; Dacremont, G. A.; Van Costerm, R. N. Am. J. Hum. Genet. 2000, 66, 1744. Greimel, P.; Spreitz, A. E.; Wrodnigg, T. M. Curr. Top. Med. Chem. 2003, 3, 513-523.
3. Fernandes, B.; Sagman, U.; Augur, M.; Demetrio, M.; Dennis, J. W. Cancer Res. 1991, 51, 718. Goss, P. E.; Reid, C. L.; Bailey, D.; Dennis, J. W. Clin. Cancer Res. 1997, 3, 1077-1086. Fiaux, H.; Popowycz, F.; Favre, S.; Schutz, C.; Vogel, P.; Gerber-Lemaire, S.; Juillerat-Jeanneret, L. J. Med. Chem. 2005, 48, 4237-4246. Stutz., A. E. (Ed.), Iminosugars as Glycosidase Inhibitors: Nojirimycin and Beyond, Wiley—VCH, Weinheim, 1999.
4. Holman, R. R.; Cull, C. A.; Turner, R. C. Diabetes Care, 1999, 22, 960-964.
5. Matsumoto, K.; Yano, M.; Miyake, S.; Ueki, Y.; Yamaguchi, Y.; Akazawa, S.; Tominaga, Y. Diabetes Care 1998, 21, 256-260.
6. Cox, T.; Lachmann, R.; Hollak, C.; Aerts, J.; van Weely, S.; Hrebicek, M.; Platt, F.; Butters, T.; Dwek, R.; Moyses, C.; Gow, I.; Elstein, D.; Zimran, A. Lancet 2000, 355, 1481-1485.
7. Koshland, D. E. Biol. Rev. 1953, 28, 416-436. McCarter, J. D.; Withers, S. G. Curr. Opin. Struct. Biol. 1994, 4, 885-892.
8. Dwek, R. A. Chem. Rev. 1996, 96, 683-720.
9. Sinnott, M. L. Chem. Rev. 1990, 90, 1171-1202. Heightman, T. D.; Vasella, A. T. Angew. Chem. Int. Ed. 1999, 38, 750-770. Zechel, D. L.; Withers, S. G. Acc. Chem. Res. 2000, 33, 11-18.
10. Mohan, S.; Pinto, B. M. Carbohydr. Res. 2007, 342, 1551-1580.
11. Yoshikawa, M.; Xu, F.; Nakamura, S.; Wang, T.; Matsuda, H.; Tanabe, G.; Muraoka, O. Heterocycles 2008, 75, 1397-1405.
12. Yoshikawa, M.; Murakami, T.; Shimada, H.; Matsuda, H.; Yomahara, J.; Tanabe, G.; Muraoka, O. Tetrahedron Lett. 1997, 38, 8367-8370.
13. Yoshikawa, M.; Murakami, T.; Yashiro, K.; Matsuda, H.; Chem. Pharm. Bull. 1998, 46, 1339-1340.
14. (a) Ozaki, S.; Oe, H. Kitamura, S. J. Nat. Prod. 2008, 71, 981-984. (b) Oe, H.; Ozaki, S. Biosci. Biotechnol. Biochem, 2008, 72, 1962-1964.
15. Muraoka, O.; Xie, W.; Tanabe, G.; Amer, M. F. A.; Minematsu, T.; Yoshikawa, M. Tetrahedron Lett. 2008, 49, 7315-7317.
16. Jayawardena, M. H. S.; de Alwis, N. M. W.; Hettigoda, V.; Fernando, D. J. S. J. Ethnopharmacol. 2005, 97, 215-218.
17. For a review: Mohan, S.; Pinto, B. M. Carbohydr. Res. 2007, 342, 1551-1580.
18. Johnston, B. D.; Jensen, H. H.; Pinto, B. M.: J. Org. Chem. 2006, 71, 1111-1118.
19. Liu, H.; Nasi, R.; Jayakanthan, K.; Sim, L.; Heipel, H.; Rose, D. R.; Pinto, B. M. J. Org. Chem. 2007, 72, 6562-6572.
20. (a) Liu, H.; Pinto, B. M. Can. J. Chem. 2006, 84, 1351-1362. (b) Liu, H.; Sim, L.; Rose. D. R.; Pinto, B. M. J. Org. Chem. 2006, 71, 3007-3013.
21. Nasi, R.; Sim, L.; Rose, D. R.; Pinto, B. M. J. Org. Chem. 2007, 72, 180-186.
22. Rossi, E. J.; Sim, L.; Kuntz, D. A.; Hahn, D.; Johnston, B. D.; Ghavami, A.; Szczepina, M. G.; Kumar, N. S.; Strerchi, E. E.; Nichols, B. L.; Pinto, B. M.; Rose, D. R. FEBS J. 2006, 273, 2673-2683.
23. (a) Tanabe, G.; Matsuoka, K.; Minematsu, T.; Morikawa, T.; Ninomiya, K.; Matsuda, H.; Yoshikawa, M.; Murata, H.; Muraoka, O.; J. Pharm. Soc. Jpn. 2007, 127, Suppl. 4, 129-130. (b) Muraoka, O.; Tanabe, G. World Intellectual Property Organization Patent, Pub. No. WO/2008/082017, International Application No. PCT/JP2008/050223, 2008.
24. Asada, M.; Kawahara, Y.; Kitamura, S. US Patent application 20070037870, Feb. 15, 2007.
25. Ley, S. V.; Baeschlin, D. K.; Dixon, D. J.; Foster, A. C.; Ince, S. J.; Priepke, H. W. M.; Reynolds, D. J. Chem. Rev. 2001, 101, 53-80 and the references within.; Hense, A.; Ley, S. V.; Osborn, H. M. I.; Owen, D. R.; Poisson, J.-F.; Warriner, S. L.; Wesson, K. E. J. Chem. Soc., Perkin Trans. 1, 1997, 2023-2031.
26. Liu, H.; Nasi, R.; Jayakanthan, K.; Heipel, H.; Sim. L.; Rose, D. R.; Pinto, B. M. J. Org. Chem. 2007, 72, 6562-6572.
27. Cha, J. K.; Christ, W. J.; Kishi, Y. Tetrahedron 1984, 40, 2247.; Cha, J. K.; Kim, N.-S. Chem. Rev. 1995, 95, 1761-1795.
28. Harris, J. M.; Mark D. Keranen, M. D.; O'Doherty, G. A. 1999, 64, 2982-2983.
29. See: tentative rules for carbohydrate nomenclature. Biochemistry, 1971, 10, 3983-4004.
30. Ghavami, A.; Sadalapure, K. S.; Johnston, B. D.; Lobera, M.; Snider, B. B.; Pinto, B. M. Synlett 2003, 1259-1262.
31. Liu, H.; Pinto, B. M. J. Org. Chem. 2005, 70, 753-755.
32. Baggett, N.; Stribblehill, P. J. Chem. Soc., Perkin Trans. 1, 1977, 1123-1126.
33. Kim, K. S.; Szarek, W. A. Synthesis 1978, 48-50.
34. Grindley, T. B.; Szarek, W. A. Can. J. Chem. 1974, 52, 4062-4070.
35. Harris. J. M.; Keranen, M. D.; O'Doherty, G. A. J. Org. Chem. 1999, 64, 2982-2983
36. Jacobsen, E. N.; Markó, I.; Mungall, W. S.; Schröder, G.; Sharpless, K. B. J. Am. Chem. Soc. 1988, 110, 1968-1970.
37. Ghavami, A.; Sadalapur, K. S.; Johnston, B. D.; Lobera, M.; Snider, B. B.; Pinto, B. M. Synlett 2003, 1259-1262.
38. (a) Montgomery, E. M.; Hudson, C. S. J. Am. Chem. Soc. 1939, 61, 1654-1658. (b) Nordal, A.; Benson, A. A. J. Am. Chem. Soc. 1954, 76, 5054-5055. (c) Jones, J. K. N.; Wall, R. A. Nature 1961, 189, 746-746.
39. Ness, A. T.; Hann. R. M.; Hudson, C. S. J. Am. Chem. Soc. 1948, 70, 765-770.
40. Nasi, R.; Patrick, B. O.; Sim, L.; Rose, D. R.; Pinto, B. M. J. Org. Chem. 2008, 73, 6172-6181.
41. Sim, L.; Rose. D. R. unpublished data.

1. A compound having the general structure I 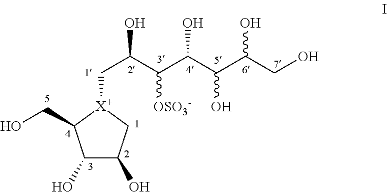
wherein X is selected from the group consisting of S, Se and NH, excluding naturally occurring kotalanol having the structure II

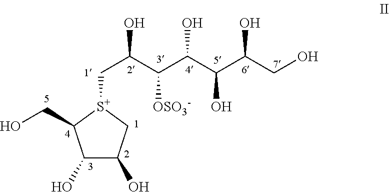

2. A compound having the general structure III 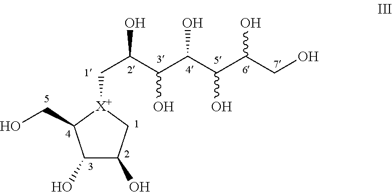
wherein X is selected from the group consisting of S, Se and NH, excluding naturally occurring de-O-sulfonated kotalanol having the structure IV

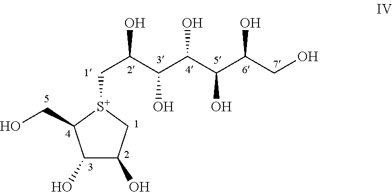

3. A compound as defined in claim 1, wherein the stereochemistry at carbon C-5′ is S—, and the stereochemistry at carbon C-6′ is S— or R—.

4. A compound as defined in claim 1, wherein the stereochemistry at carbon C-5′ is S—, the stereochemistry at carbon C-6′ is S— or R—, and wherein X is S.

5. A compound as defined in claim 1, wherein the stereochemistry at carbon C-5′ is R—, and the stereochemistry at carbon C-6′ is R—.

6. A compound as defined in claim 1, wherein the stereochemistry at carbon C-5′ is R—, the stereochemistry at carbon C-6′ is S—, and wherein X is Se.

7. A compound as defined in claim 1, wherein the stereochemistry at carbon C-5′ is R—, the stereochemistry at carbon C-6′ is S—, and wherein X is NH.

8. A compound as defined in claim 1, having the structure V, VI or VII 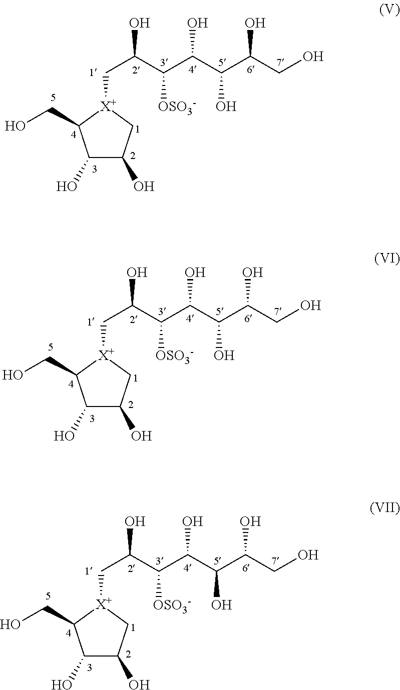
wherein X is selected from the group consisting of S, Se or NH.

9. A compound as defined in claim 8 having the structure V or VI, wherein X is S.

10. A compound as defined in claim 1, having the structure VIII 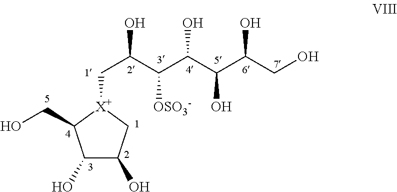
wherein X is selected from the group consisting of Se or NH.

11. A compound as defined in claim 2, having the structure IX, X or XI 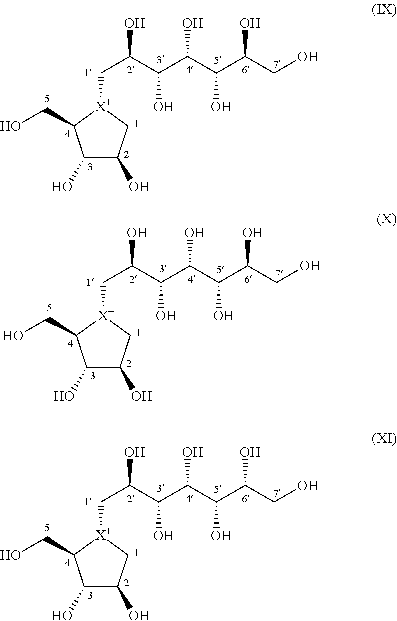
wherein X is selected from the group consisting of S, Se or NH.

12. A compound as defined in claim 2, having the structure XII 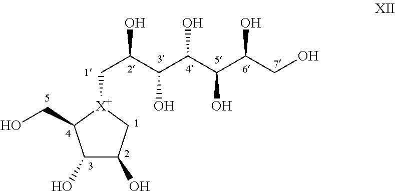
wherein X is selected from the group consisting of Se or NH.

13. A method for synthesizing a compound having the general formula XIII 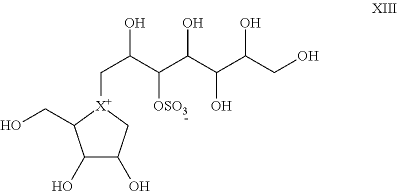
comprising the steps set forth in Scheme I

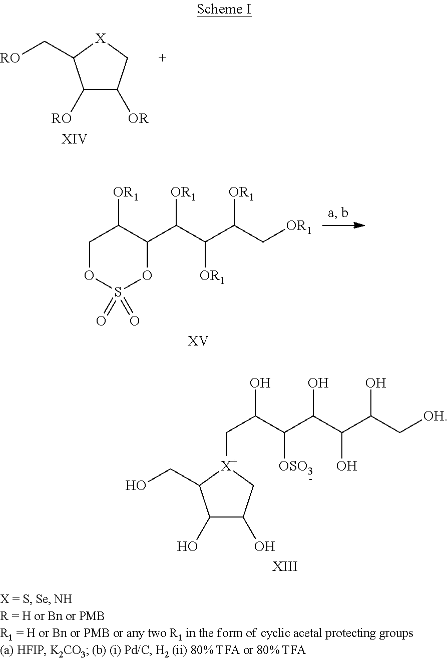

14. A method according to claim 13 for synthesizing a compound having the general formula XVI 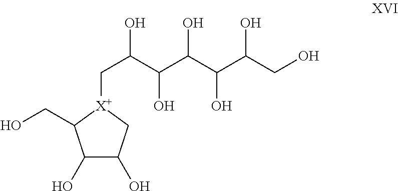
further comprising the step of de-O-sulfonation of a compound having the general formula XIII using 5% methanolic HCl.

15. A method according to claim 13 for synthesizing a compound having the general formula I 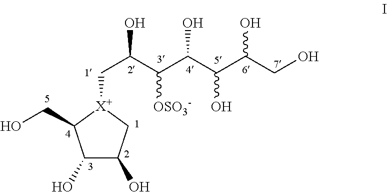
comprising reacting a 5-membered sugar of the general formula XVII 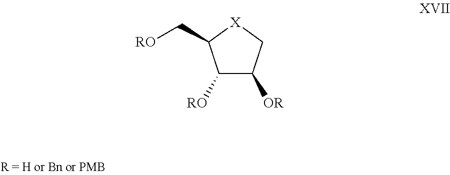
wherein X is selected from the group consisting of S, Se and NH, with a cyclic sulfate of the general formula XVIII

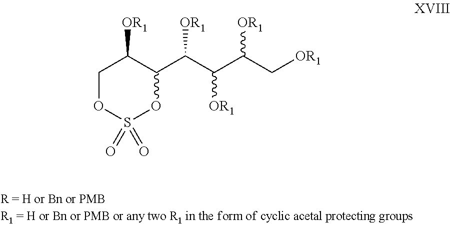

16. A method according to claim 15, wherein the 5-membered sugar is 1,4-anhydro-4-thio-D-arabinitol, 1,4-anhydro-4-seleno-D-arabinitol, or 1,4-dideoxy-1,4-imino-D-arabinitol.

17. A method according to claim 15, wherein the cyclic sulfate is 1,2,6-tri-O-benzyl-3,4-O-(2′, 3′-dimethoxybutane-2′,3′-diyl)-D-glycero-D-gulitol-5,7-cyclic sulfate or 2,6,7-tri-O-benzyl-4,5-O-(2′,3′-dimethoxybutane-2′,3′-diyl)-D-glycero-L-gulitol-1,3-cyclic sulfate, or a compound having the structure 48 or 52

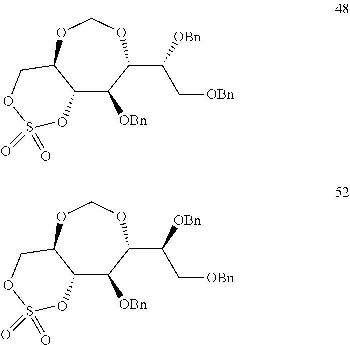

18. A method according to claim 13 for synthesizing a compound having the structure XIX 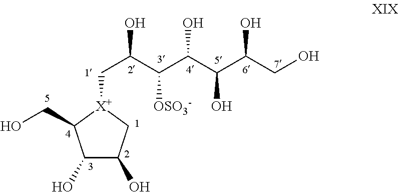
wherein X is selected from the group consisting of S, Se and NH, comprising the steps set forth in Scheme II and Scheme III

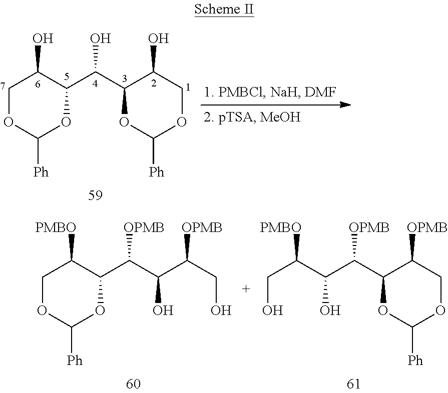

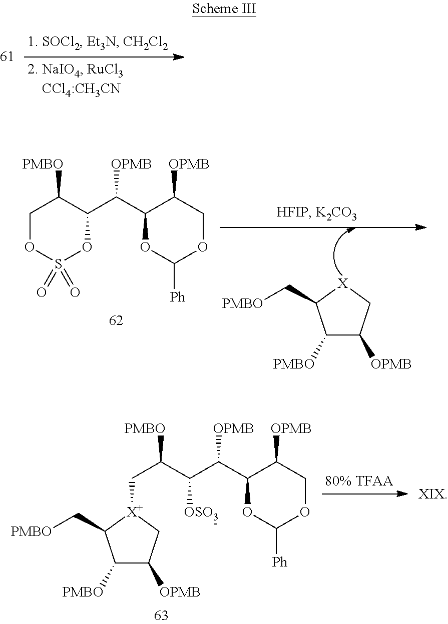

19. A method according to claim 18, wherein X is S.

20. A method according to claim 18, further comprising the step of de-O-sulfonating the compound having the structure XIX using 5% methanolic HCl.

21. A method according to claim 13 for synthesizing kotalanol having the structure XX 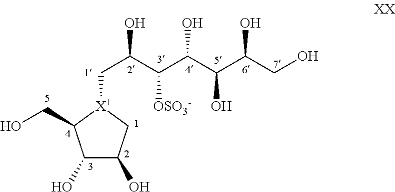
wherein the cyclic sulfate is derived from D-glycero-D-galacto-heptitol having the structure XXI

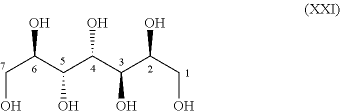

22. A method of synthesizing kotalanol having the structure II 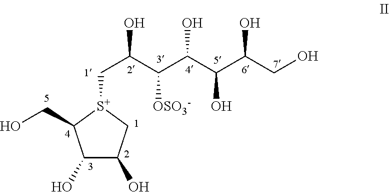
comprising (a) reacting a cyclic sulfate and a protected thioarabinitol to produce a reaction product; and
(b) deprotecting the reaction product, wherein the cyclic sulfate is derived from D-perseitol.

23. A method according to claim 22 comprising the steps set forth in Scheme IV

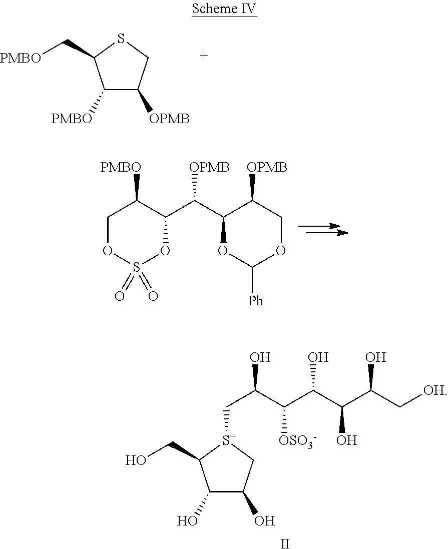

24. A method of synthesizing kotalanol having the structure II 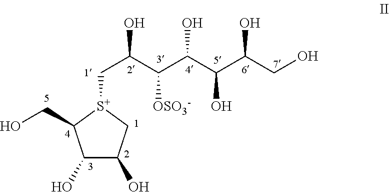
wherein D-perseitol having the structure XXI 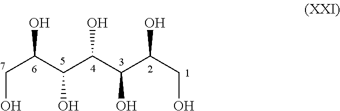
or a derivative thereof is used in the synthesis.

25. A method according to claim 13 for synthesizing compounds having the structure XXII or XXIII 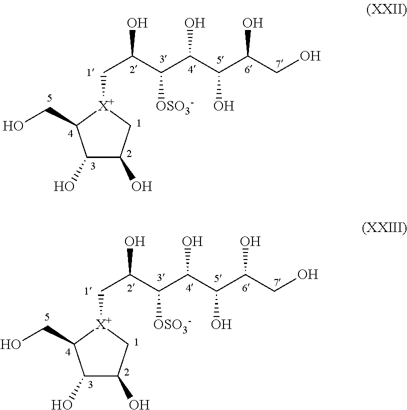
wherein X is selected from the group consisting of S, Se, and NH, comprising the steps set forth in Scheme V and Scheme VI

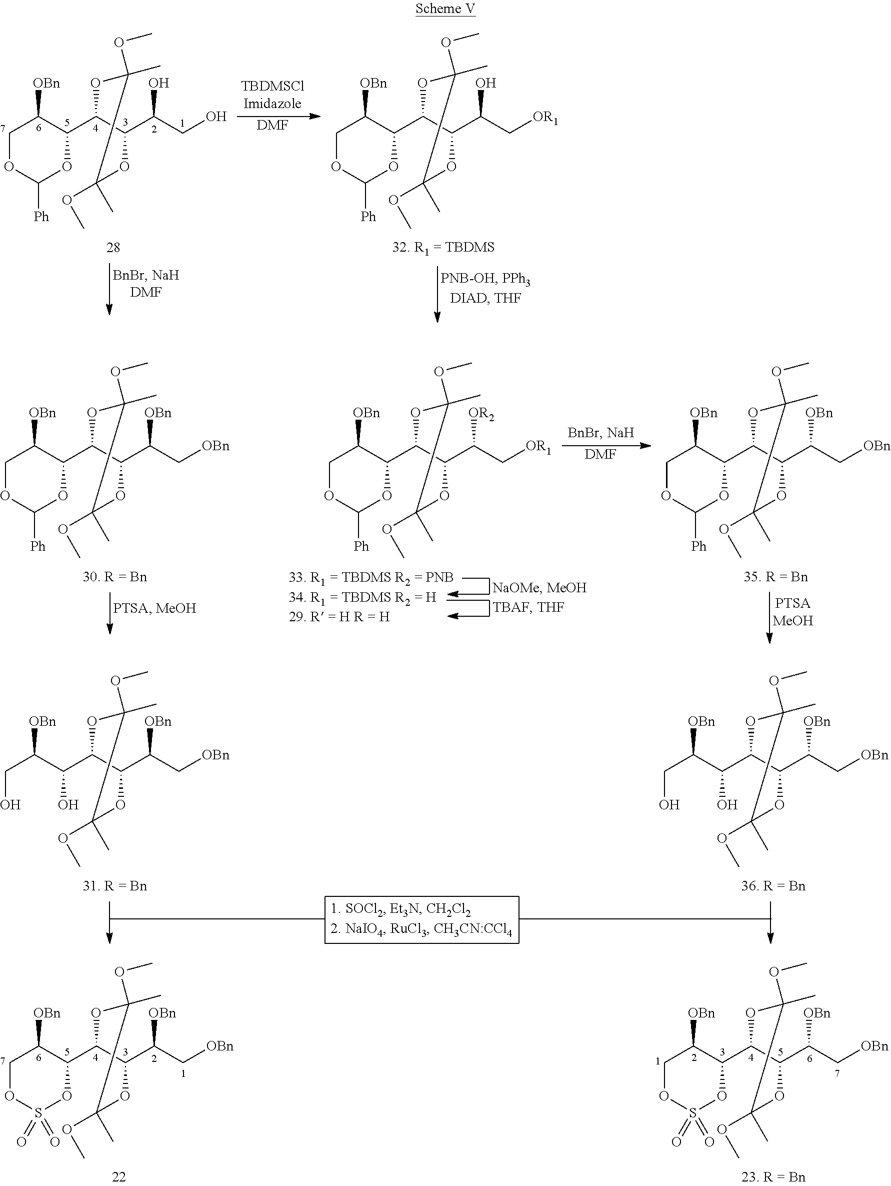

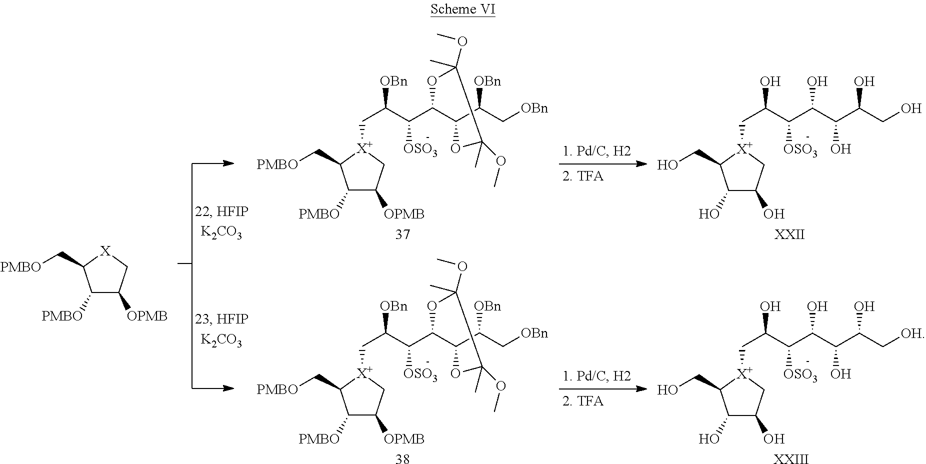

26. A method according to claim 25, wherein X is S.

27. A method according to claim 25, further comprising the step of de-O-sulfonating the compound having the structure Val or XXIII in 5% methanolic HCl.

28. A method for synthesizing a compound having the structure XXIV 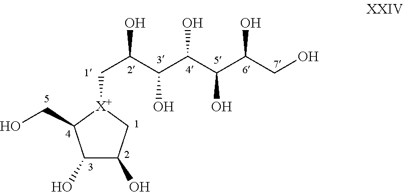
wherein X is selected from the group consisting of S, Se and NH, comprising the steps set forth in Scheme VII, Scheme VIII and Scheme IX

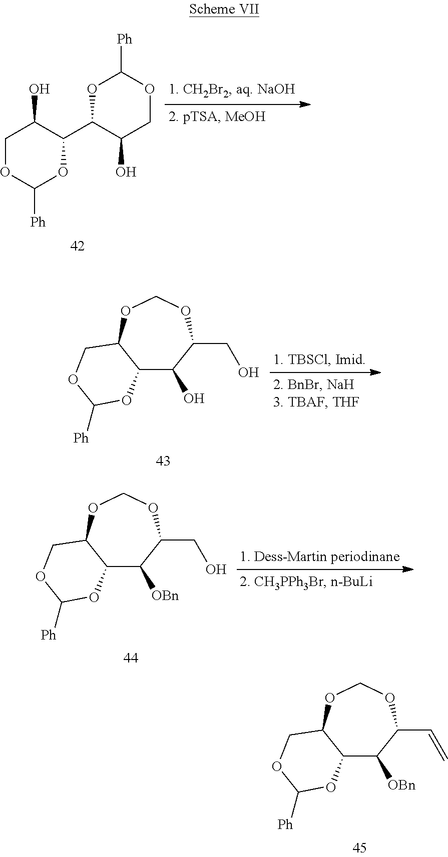

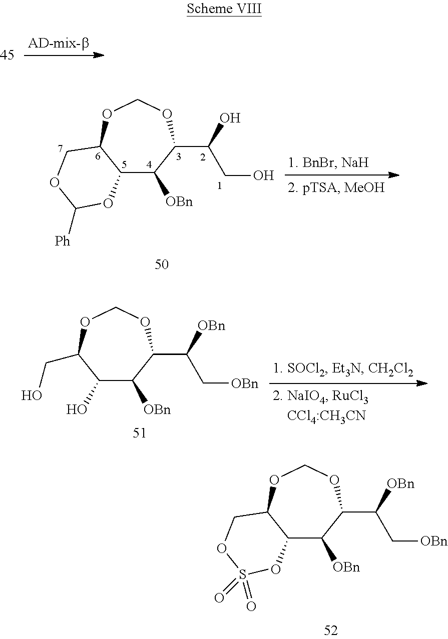

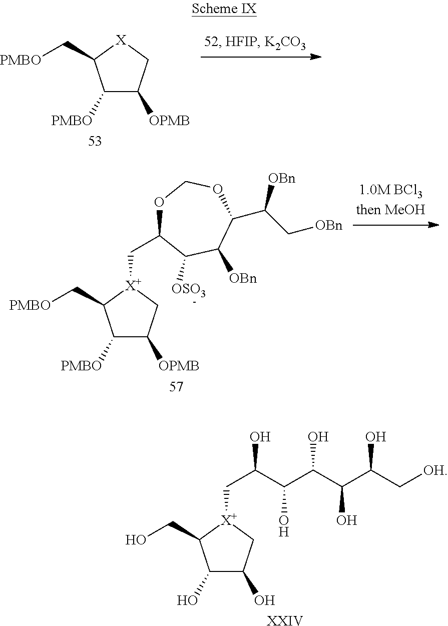

29. A method for synthesizing a compound having the structure XXV 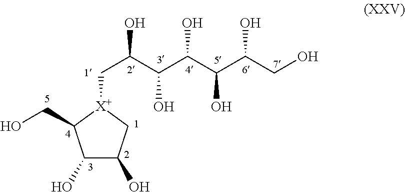
wherein X is selected from the group consisting of S, Se and NH, comprising the steps set forth in Scheme VII, Scheme X, and Scheme XI

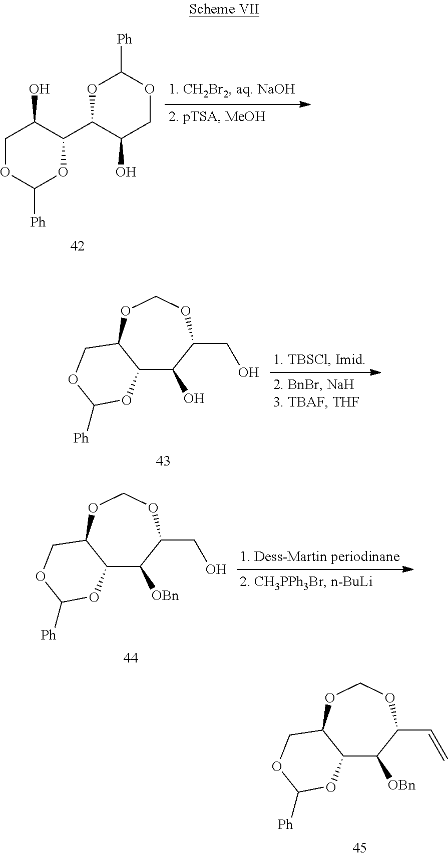

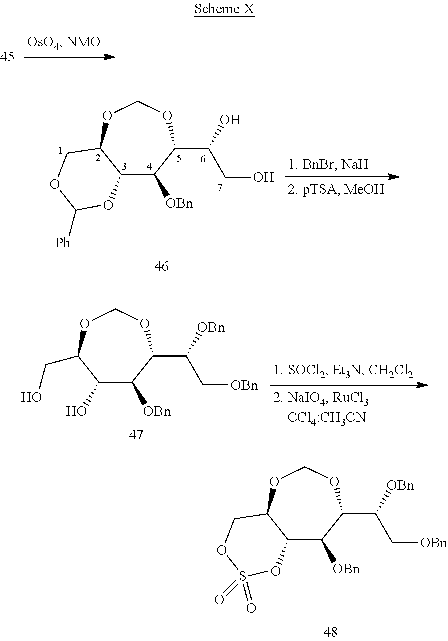

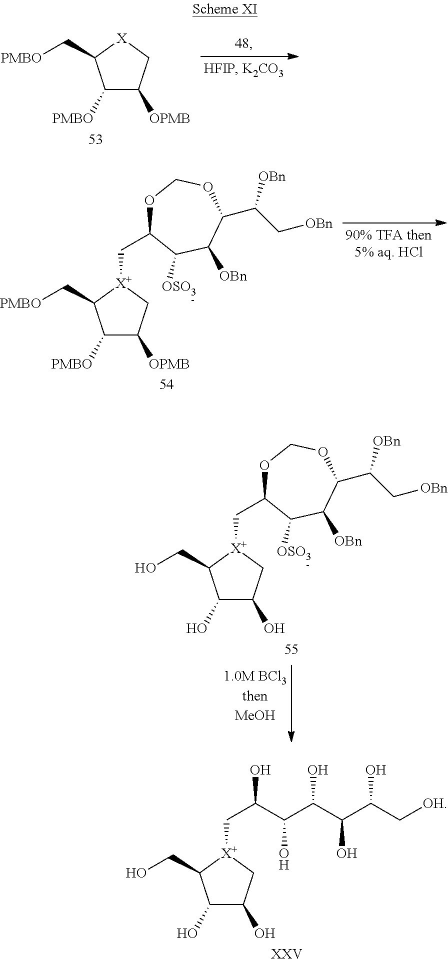

30. A method according to claim 29, wherein X is S.

31. A method for synthesizing a compound having the structure XXVI 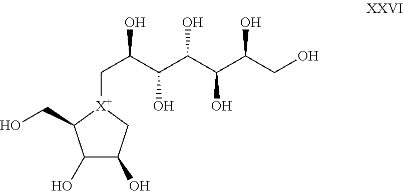
wherein X is selected from the group consisting of S, Se and NH, comprising the steps set forth in Scheme XII.

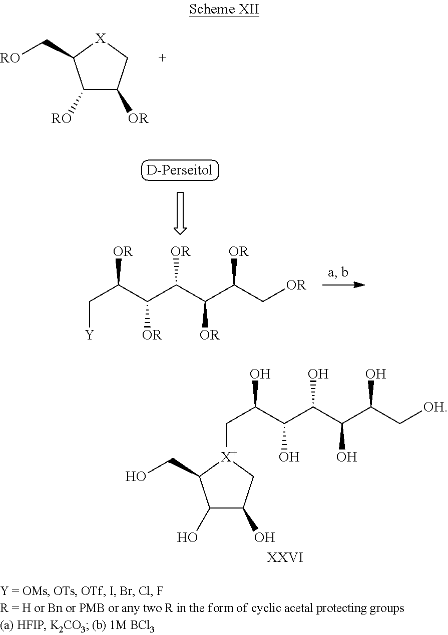

32. A method for synthesizing a compound having the chemical formula XXVII 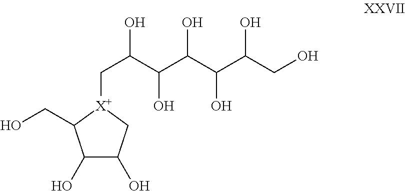
wherein X is selected from the group consisting of S, Se and NH, comprising the steps set forth in Scheme XIII

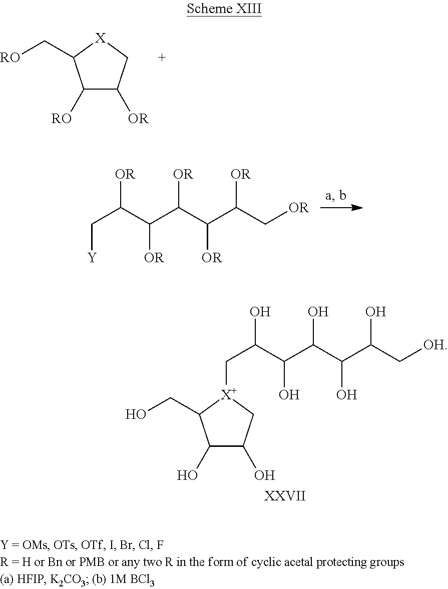

33. A method of synthesizing a compound having the general structure III 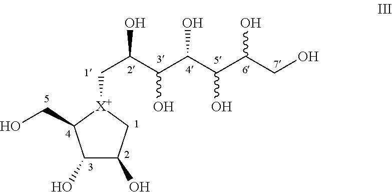
wherein X is selected from the group consisting of S, Se and NH, comprising the steps set forth in Scheme XIV

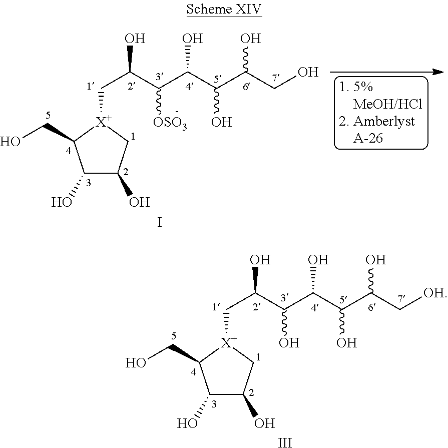

34. The use of a compound according to claim 1 as a standard to calibrate a natural product intended to be sold or used as an herbal remedy.

35. The use of a compound synthesized by the method of claim 13 to calibrate a natural product intended to be sold or used as an herbal remedy.

36. The use as defined in claim 34, wherein the compound is used as a standard in HPLC, capillary electrophoresis, NMR, or HPLC-mass spectrometry analysis of the natural product.

37. A method for treating diabetes in an affected patient comprising the step of administering to said patient a therapeutically effective amount of a compound according to claim 1.

38. A method for treating a disorder which is ameliorated by the inhibition of glycosidases by administering a therapeutically effective amount of a compound according to claim 1 to a mammal in need of such treatment.

39. A method according to claim 38, wherein the glycosidase is an intestinal glucosidase.

40. A method according to claim 39, wherein the intestinal glucosidase is maltase glucoamylase.

41. A composition comprising a compound as defined in claim 1 and a pharmaceutically effective carrier.

42. A compound as defined in claim 2, wherein the stereochemistry at carbon C-5′ is S—, and the stereochemistry at carbon C-6′ is S— or R—.

43. A compound as defined in claim 2, wherein the stereochemistry at carbon C-5′ is R—, and the stereochemistry at carbon C-6′ is R—.

44. A compound as defined in claim 2, wherein the stereochemistry at carbon C-5′ is R—, the stereochemistry at carbon C-6′ is S—, and wherein X is Se.

45. A compound as defined in claim 2, wherein the stereochemistry at carbon C-5′ is R—, the stereochemistry at carbon C-6′ is S—, and wherein X is NH.

46. A method for treating diabetes in an affected patient comprising the step of administering to said patient a therapeutically effective amount of a compound according to claim 2.

47. A method for treating a disorder which is ameliorated by the inhibition of glycosidases by administering a therapeutically effective amount of a compound according to claim 2 to a mammal in need of such treatment.

48. A composition comprising a compound as defined in claim 2 and a pharmaceutically effective carrier.
